(19) United States
(12) Patent Application Publication
(10) Pub. No.: US 2025/0163173 A1
(43) Pub. Date: May 22, 2025

(54) MONOVALENT BLOOD BRAIN BARRIER SHUTTLE MODULES

(71) Applicant: Hoffmann-La Roche Inc., Little Falls, NJ (US)

(72) Inventors: Petra RUEGER, Penzberg (DE); Georg TIEFENTHALER, Sindelsdorf (DE); Ekkehard MOESSNER, Kreuzlingen (CH); Jens NIEWOEHNER, Muenchen (DE); Adrian HUGENMATTER, Zürich (CH); Cuiying SHAO, Shanghai (CN); Francesca ROS, Bernried (DE); Gang XU, Shanghai (CN)

(73) Assignee: Hoffmann-La Roche Inc., Little Falls, NJ (US)

(21) Appl. No.: 19/022,791

(22) Filed: Jan. 15, 2025

Related U.S. Application Data
(63) Continuation of application No. 16/457,267, filed on Jun. 28, 2019.
Application No. 16/457,267 is a division of application No. 15/203,057, filed on Jul. 6, 2016, now Pat. No. 10,364,292.
Application No. 15/203,057 is a continuation of application No. PCT/EP2014/079353, filed on Dec. 29, 2014.

(30) Foreign Application Priority Data
Jan. 6, 2014 (WO) PCT/CN2014/070183

(51) Int. Cl. C07K 16/28; A61K 39/00; C07K 16/18

(52) U.S. Cl. CPC C07K 16/2881; C07K 16/18; A61K 2039/505; C07K 2317/31; C07K 2317/35; C07K 2317/55; C07K 2317/56; C07K 2317/622; C07K 2319/33

(57) ABSTRACT

Herein is reported a blood brain barrier shuttle module comprising a brain effector entity, a linker and one monovalent binding entity which binds to a blood brain barrier receptor, wherein the linker couples the effector entity to the monovalent binding entity which binds to the blood brain barrier receptor wherein the monovalent binding entity does not comprise the variable domains of the anti-transferrin receptor antibody 8D3 (SEQ ID NO: 01 and SEQ ID NO: 02) or of the variant anti-transferrin receptor antibody 8D3v (SEQ ID NO: 01 and SEQ ID NO: 03).

### CROSS REFERENCE TO RELATED APPLICATIONS

This application is a continuation of U.S. patent application Ser. No. 16/457,267, filed Jun. 28, 2019 which is a division of U.S. patent application. Ser. No. 15/203,057, filed Jul. 6, 2016, now U.S. Pat. No. 10,364,292, granted on Jul. 30, 2019 which is a continuation of International Application No. PCT/EP2014/079353, having an international filing date of Dec. 29, 2014, which claims benefit to International Application No. PCT/CN2014/070183, filed Jan. 6, 2014, the entire contents of which are incorporated herein by reference.

### SEQUENCE LISTING

This application contains a Sequence Listing which has been submitted electronically in XML format and is hereby incorporated by reference in its entirety. Said XML copy, created on Jan. 14, 2025 is named P31922-US-2_Seq_List.xml and is 160,272 bytes in size.

### FIELD OF THE INVENTION

The present invention relates to a blood brain barrier shuttle module that has one binding specificity that specifically binds to a blood brain barrier receptor (BBBR) and which is monovalent with respect to this binding specificity and methods of using this construct as blood barrier shuttle and in the treatment of neurological disorders.

### BACKGROUND

Brain penetration of neurological disorder drugs such alls e.g. large biotherapeutic drugs or small molecule drugs having a low brain penetration, is strictly limited by the extensive and impermeable blood brain barrier (BBB) together with the other cell component in the neurovascular unit (NVU). Many strategies to overcome this obstacle have been tested and one is to utilize transcytosis pathways mediated by endogenous receptors expressed on the brain capillary endothelium (blood brain barrier receptor). Recombinant proteins such as monoclonal antibodies or peptides have been designed against these receptors to enable receptor-mediated delivery of biotherapeutics to the brain. However, strategies to maximize brain uptake while minimizing miss-sorting within the brain endothelial cells (BECs), and the extent of accumulation within certain organelles (especially organelles that lead to degradation of the biotherapeutic) in BECs, remain unexplored.

Monoclonal antibodies and other biotherapeutics have huge therapeutic potential for treatment of pathology in the central nervous system (CNS). However, their route into the brain is prevented by the BBB. Previous studies have illustrated that a very small percentage (approximately 0.1%) of an IgG injected in the bloodstream is able to penetrate into the CNS compartment (Felgenhauer, Klin. Wschr. 52 (1974) 1158-1164). This will certainly limit any pharmacological effect due to the low concentration within CNS of the antibody.

Therefore, there is a need for delivery systems of neurological disorder drugs across the BBB to shuttle the drugs into the brain efficiently.

In WO 2014/033074 a blood brain barrier shuttle is reported.

Mouse 8D3 anti-transferrin antibody and variable light chain domain (VL) variant (L596V and L598I) thereof is reported by Boado, R. J., et al. (Biotechnol. Bioeng. 102 (2009) 1251-1258).

### SUMMARY

One aspect of the current invention is a blood brain barrier shuttle module comprising a brain effector entity, a linker and one monovalent binding entity which binds to a blood brain barrier receptor, wherein the linker couples the effector entity to the monovalent binding entity, which binds to the blood brain barrier receptor, wherein the monovalent binding entity does not comprise the variable domains of the anti-transferrin receptor antibody 8D3 (SEQ ID NO: 01 and SEQ ID NO: 02) or of the variant anti-transferrin receptor antibody 8D3v (SEQ ID NO: 01 and SEQ ID NO: 03).

The anti-transferrin receptor antibody 8D3 has a heavy chain variable domain with the following amino acid sequence:

(SEQ ID NO: 01)
EVQLVESGGG LVQPGNSLTL SCVASGFTFS NYGMHWIRQA
PKKGLEWIAM IYYDSSKMNY ADTVKGRFTI SRDNSKNTLY
LEMNSLRSED TAMYYCAVPT SHYVVDVWGQ GVSVTVSS.

The anti-transferrin receptor antibody 8D3 has a light chain variable domain with the following amino acid sequence:

(SEQ ID NO: 02)
DIQMTQSPAS LSASLEEIVT ITCQASQDIG NWLAWYQQKP
GKSPQLLIYG ATSLADGVPS RFSGSRSGTQ FSLKISRVQV
EDIGIYYCLQ AYNTPWTFGG GTKLELK.

The variant anti-transferrin receptor antibody 8D3v has the same heavy chain variable domain as antibody 8D3 and a light chain variable domain with mutants L104V and L106I that has the following amino acid sequence:

(SEQ ID NO: 03)
DIQMTQSPAS LSASLEEIVT ITCQASQDIG NWLAWYQQKP
GKSPQLLIYG ATSLADGVPS RFSGSRSGTQ FSLKISRVQV
EDIGIYYCLQ AYNTPWTFGG GTKVEIK.

In one embodiment of the blood brain barrier shuttle module the monovalent binding entity which binds to the blood brain barrier receptor is a polypeptide.

In one embodiment of the blood brain barrier shuttle module the monovalent binding entity which binds to the blood brain barrier receptor comprises a molecule selected from the group consisting of a blood brain barrier receptor ligand, a full length antibody, a scFv, an Fv, a scFab, and a VHH.

In one embodiment of the blood brain barrier shuttle module the blood brain receptor is selected from the group consisting of transferrin receptor, insulin receptor, insulin-like growth factor receptor, low density lipoprotein receptor-related protein 8, low density lipoprotein receptor-related protein 1 and heparin-binding epidermal growth factor-like growth factor. In one embodiment the blood brain receptor is the transferrin receptor.

In one embodiment the monovalent binding entity specifically binds to human transferrin receptor and to cynomolgus transferrin receptor.

In one embodiment of the blood brain barrier shuttle module the monovalent binding entity which binds to the blood brain barrier receptor comprises one scFab directed to the transferrin receptor, more particularly, a scFab that specifically binds to an epitope in the transferrin receptor comprised within the amino acid sequence of SEQ ID NOs: 04, 05, or 06.

In one embodiment of the blood brain barrier shuttle module the monovalent binding entity which binds to the blood brain barrier receptor comprises one scFv directed to the transferrin receptor, more particularly, a scFv recognizing an epitope in the transferrin receptor comprised within the amino acid sequence of SEQ ID NOs: 04, 05, or 06.

In one embodiment of the blood brain barrier shuttle module the brain effector entity is selected from the group consisting of neurological disorder drugs, neurotrophic factors, growth factors, enzymes, cytotoxic agents, antibodies directed to a brain target, monoclonal antibodies directed to a brain target, peptides directed to a brain target.

In one embodiment of the blood brain barrier shuttle module the brain target is selected from the group consisting of β-secretase 1, Aβ (Abeta), epidermal growth factor, epidermal growth factor receptor 2, tau, phosphorylated tau, phosphorylated tau(pS422), apolipoprotein E4, alpha synuclein, oligomeric fragments of alpha synuclein, CD20, huntingtin, prion protein, leucine rich repeat kinase 2, parkin, presenilin 2, gamma secretase, death receptor 6, amyloid precursor protein, p75 neurotrophin receptor and caspase 6.

In a particular embodiment of the blood brain barrier shuttle module the brain effector entity is a polypeptide.

In one embodiment of the blood brain barrier shuttle module the monovalent binding entity which binds to the blood brain receptor is a polypeptide and the monovalent binding entity is conjugated to the C-terminal end of the brain effector entity either directly or via a linker.

In one embodiment of the blood brain barrier shuttle module the brain effector entity comprises a full length antibody directed to a brain target. In one embodiment the full length antibody is an IgG.

In one preferred embodiment of the blood brain barrier shuttle module the blood brain barrier shuttle comprises a full length IgG antibody as brain effector entity, a linker and one scFab as the monovalent binding entity which binds the blood brain barrier receptor, wherein the scFab is conjugated to the C-terminal end of the Fc-region of one of the heavy chains of the IgG antibody via the linker.

In one embodiment of the blood brain barrier shuttle module the first heavy chain of the antibody of the blood brain barrier shuttle directed to a brain target comprises a first dimerization module and the second heavy chain of the antibody of the blood brain barrier shuttle to a brain target comprises a second dimerization module allowing heterodimerization of the two heavy chains.

In one embodiment of the blood brain barrier shuttle module the first dimerization module of the first heavy chain of the antibody of the blood brain barrier shuttle directed to the brain target comprises knobs and the dimerization module of the second heavy chain of the antibody of the blood brain barrier shuttle directed to the brain target comprises holes according to the knobs-into-holes strategy.

In one embodiment of the blood brain barrier shuttle module the linker is a peptidic linker. In one embodiment the peptidic linker has an amino acid sequence with a length of at least 25 amino acids. In one embodiment the peptidic linker has an amino acid sequence with a length of 30 to 50 amino acids. In one embodiment the peptidic linker is (G4S)6G2 (SEQ ID NO: 07) or (G4S)4 (SEQ ID NO: 08).

The following three embodiments are directed to a blood brain barrier shuttle module wherein the brain effector entity is a polypeptide with the proviso that the brain effector entity is not a full length antibody, in particular not a full length IgG.

In one embodiment of the blood brain barrier shuttle module the monovalent binding entity which binds to the blood brain barrier receptor comprises a CH2-CH3 Ig entity and one scFab (comprising a first linker), which binds to the blood brain barrier receptor, wherein the scFab is coupled to a C-terminal end of the CH2-CH3 Ig entity by a second linker.

In one embodiment of the blood brain barrier shuttle module the blood brain barrier shuttle comprises a brain effector entity, a linker, a CH2-CH3 Ig domain, a second linker and one scFab, which binds to the blood brain barrier receptor, wherein the brain effector entity is conjugated by a first linker to an N-terminal end of the CH2-CH3 Ig domain and the scFab is conjugated to a C-terminal end of the CH2-CH3 Ig domain by a second linker.

In one embodiment of the blood brain barrier shuttle module the CH2-CH3 Ig entity is a CH2-CH3 IgG entity.

Further aspects of the current invention are an (isolated) nucleic acid encoding the blood brain barrier shuttle module as reported herein, a host cell comprising the (isolated) nucleic acid encoding the blood brain barrier shuttle module, and a pharmaceutical formulation comprising the blood brain barrier shuttle module.

The blood brain barrier shuttle module as reported herein can be used as a medicament, in particular it can be used for the treatment of a neurological disorder such as e.g. Alzheimer's disease.

The blood brain barrier shuttle module as reported herein can be used to transport the brain effector entity across the blood brain barrier.

In a particular embodiment, the heavy chain of the IgG antibody of the blood brain barrier shuttle module as reported herein conjugated at its C-terminal end of the Fc-region to the scFab as monovalent binding entity, which binds to the blood brain barrier receptor, has the following structure:
IgG heavy chain,
linker conjugating the C-terminal end of the Fc-region of the IgG heavy chain to the N-terminal end of the VL domain of the scFab,
variable light chain domain (VL) and C-kappa light chain domain of the scFab,
linker conjugating the C-terminal end of the C-kappa light chain domain of the scFab to the N-terminal end of the VH domain of the scFab,
variable heavy chain domain (VH) of the scFab antibody and IgG CH1 heavy chain domain.

One aspect as reported herein is a fusion polypeptide to transport a brain effector entity across the blood brain barrier comprising a CH2-CH3 Ig entity, a linker and one scFab that specifically binds to a blood brain barrier receptor, wherein the scFab is conjugated to a C-terminal end of the CH2-CH3 Ig entity by the linker, wherein scFab does not comprise the variable domains of the anti-transferrin receptor antibody 8D3 (SEQ ID NO: 01 and SEQ ID NO: 02) or of the variant anti-transferrin receptor antibody 8D3v (SEQ ID NO: 01 and SEQ ID NO: 03).

One aspect as reported herein is a fusion polypeptide to transport a brain effector entity across the blood brain barrier comprising a CH2-CH3 Ig entity, a linker and one scFv that specifically binds to a blood brain barrier receptor, wherein the scFv is conjugated to a C-terminal end of the CH2-CH3 Ig entity by the linker, wherein scFv does not comprise the variable domains of the anti-transferrin receptor antibody 8D3 (SEQ ID NO: 01 and SEQ ID NO: 02) or of the variant anti-transferrin receptor antibody 8D3v (SEQ ID NO: 01 and SEQ ID NO: 03).

In one embodiment the fusion polypeptide further comprises a linker at the N-terminal end of the CH2-CH3 Ig entity to conjugate the brain effector entity to the N-terminal end of the CH2-CH3 Ig entity.

In one embodiment of the fusion polypeptide the brain effector entity is selected from the group consisting of neurological disorder drugs, neurotrophic factors, growth factors, enzymes, cytotoxic agents, antibody fragments or peptides directed to a brain target selected from the group consisting of scFv, Fv, scFab, Fab, VHH, F(ab′)2.

In one embodiment of the fusion polypeptide the scFab or the scFv that specifically binds to the blood brain barrier receptor specifically binds to the transferrin receptor. In one embodiment the scFab or the scFv specifically binds to an epitope of the transferrin receptor comprised within the amino acid sequence of SEQ ID NO: 04, 05 or 06.

In embodiment of the fusion polypeptide the linker is a peptidic linker. In one embodiment the peptidic linker has an amino acid sequence with a length of at least 15 amino acids. In one embodiment the peptidic linker has a length of 20 to 50 amino acids. In one embodiment the peptidic linker has the amino acid sequence (G4S)6G2, (SEQ ID NO: 07) or (G4S)4 (SEQ ID NO: 08).

In one embodiment of the fusion polypeptide the CH2-CH3 Ig entity is a CH2-CH3 IgG entity.

Further aspects of the current invention are an isolated nucleic acid encoding the fusion polypeptide as reported herein and a host cell comprising the nucleic acid encoding the fusion polypeptide as reported herein.

One aspect as reported herein is a conjugate comprising a fusion polypeptide as reported herein and a brain effector entity conjugated to an N-terminal end of the CH2-CH3 Ig entity of the fusion polypeptide as reported herein via a linker.

In one embodiment of the conjugate the brain effector entity is a neurotrophic factor and the linker conjugating the neurotrophic factor to the N-terminal end of the CH2-CH3 Ig entity is a peptidic linker.

Further aspects as reported herein are a pharmaceutical formulation comprising the conjugate as reported herein and a pharmaceutical carrier, the use of the conjugate as reported herein, in particular the use of the conjugate for the treatment of a neurodegenerative disorder in particular Alzheimer's disease.

The monovalent binding entity that specifically binds to a blood brain barrier receptor can be conjugated to any terminus of the light or heavy chain of the antibody either directly or via a peptidic linker. In one embodiment the monovalent binding entity is conjugated to a C-terminus of the heavy chain.

The C-terminus of a heavy chain of an antibody can be a complete C-terminus ending with the amino acid residues PGK. The C-terminus of the heavy chain can be a shortened C-terminus in which one or two of the C-terminal amino acid residues have been removed. In one embodiment the C-terminus of the heavy chain is a shortened C-terminus ending with the amino acid residues PG.

The monovalent binding entity can be conjugated to the respective antibody chain either directly or via a peptidic linker. In one embodiment the peptidic linker has the amino acid sequence GGSGGGGSGGGGSGGGGS (SEQ ID NO: 09).

The monovalent binding entity can be an antibody scFv fragment. In one embodiment the monovalent binding entity is a scFv comprising in N- to C-terminal order a light chain variable domain—a light chain constant domain—a peptidic linker—a heavy chain variable domain—the heavy chain constant domain 1.

In one embodiment the monovalent binding entity is a scFv fragment of an anti-transferrin receptor-antibody with a (G4S)6 peptidic linker (SEQ ID NO: 10).

In one embodiment the blood-brain-barrier receptor is selected from the group consisting of transferrin receptor, insulin receptor, insulin-like growth factor receptor, low density lipoprotein receptor-related protein 8, low density lipoprotein receptor-related protein 1 and heparin-binding epidermal growth factor-like growth factor. In one embodiment blood-brain-barrier receptor is a human blood-brain-barrier receptor. In one embodiment the blood-brain-barrier receptor is the transferrin receptor and the antibody does not inhibit the binding of the transferrin receptor to transferrin. In one embodiment the blood-brain-barrier receptor is the human transferrin receptor.

In one embodiment the peptidic linker conjugating the monovalent binding entity to the brain effector entity is an amino acid sequence with a length of at least 15 amino acids. In one embodiment the peptidic linker has a length of 18 to 25 amino acids.

In one embodiment, the brain effector entity is a full length antibody. In one embodiment the brain effector entity is a full length antibody of the subclass IgG1 or IgG4.

In one embodiment the monovalent binding entity is an anti-blood brain barrier receptor antibody or a blood brain barrier receptor binding fragment thereof. In one embodiment, the anti-blood brain barrier receptor antibody or fragment thereof does not impair the binding of the blood brain barrier receptor to one or more of its native ligands. In another embodiment, the anti-blood brain barrier receptor antibody specifically binds to human transferrin receptor in such a manner that it does not inhibit binding of the human transferrin receptor to human transferrin.

In one embodiment the blood brain barrier shuttle module is effector silent.

In one embodiment the brain effector entity is a full length antibody comprising an Fc-region, wherein in case the Fc-region is of the human subclass IgG1 the Fc-region comprises the mutations L234A, L235A and P329G (numbering according to the EU index of Kabat), or in case the Fc-region is of the human subclass IgG4 the Fc-region comprises the mutations S228P, L235E and P329G (numbering according to the EU index of Kabat).

### BRIEF DESCRIPTION OF DRAWING

FIG. 1 depicts the assay scheme for the assay used in the transcytosis screening.

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, δ, γ, and μ, respectively.

“Effector functions” refer to those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody”, “intact antibody”, and “whole antibody” are used herein interchangeably to refer to an antibody having a structure substantially similar to a native antibody structure or having heavy chains that contain an Fc-region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat, E. A. et al., Sequences of Proteins of Immunological Interest, 5th ed., Bethesda MD (1991), NIH Publication 91-3242, Vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain which are hypervariable in sequence (“complementarity determining regions” or “CDRs”) and form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs herein include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt, T. J. et al. Kuby Immunology, 6th ed., W.H. Freeman and Co., N.Y. (2007), page 91) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “blood-brain barrier” (BBB) denotes the physiological barrier between the peripheral circulation and the brain and spinal cord which is formed by tight junctions within the brain capillary endothelial plasma membranes, creating a tight barrier that restricts the transport of molecules into the brain, even very small molecules such as urea (60 Daltons). The BBB within the brain, the blood-spinal cord barrier within the spinal cord, and the blood-retinal barrier within the retina are contiguous capillary barriers within the CNS, and are herein collectively referred to as the blood-brain barrier or BBB. The BBB also encompasses the blood-CSF barrier (choroid plexus) where the barrier is comprised of ependymal cells rather than capillary endothelial cells.

The term “central nervous system” (CNS) denotes the complex of nerve tissues that control bodily function, and includes the brain and spinal cord.

The term “blood-brain barrier receptor” (BBBR) denotes an extracellular membranelinked receptor protein expressed on brain endothelial cells which is capable of transporting molecules across the BBB or be used to transport exogenous administrated molecules. Examples of BBBR include but are not limited to transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary BBBR is the transferrin receptor (TfR).

The term “brain effector entity” denotes a molecule that is to be transported to the brain across the BBB. The effector entity typically has a characteristic therapeutic activity that is desired to be delivered to the brain. Effector entities include neurological disorder drugs and cytotoxic agents such as e.g. polypeptides and antibodies, in particular monoclonal antibodies or fragments thereof directed to a brain target.

The term “monovalent binding entity” denotes a molecule able to bind specifically and in a monovalent binding mode to a BBBR. The blood brain shuttle module and/or conjugate as reported herein are characterized by the presence of a single unit of a monovalent binding entity i.e. the blood brain shuttle module and/or conjugate of the present invention comprise exactly one unit of the monovalent binding entity. The monovalent binding entity includes but is not limited to polypeptides, full length antibodies, antibody fragments including Fab, Fab′, Fv fragments, single-chain antibody molecules such as e.g. single chain Fab, scFv. The monovalent binding entity can be, for example, a scaffold protein engineered using state of the art technologies like phage display or immunization. The monovalent binding entity can also be a polypeptide. In certain embodiments, the monovalent binding entity comprises a CH2-CH3 Ig domain and a single chain Fab (scFab) directed to a blood brain barrier receptor. The scFab is coupled to the C-terminal end of the CH2-CH3 Ig domain by a linker. In certain embodiments, the scFab is directed to the transferrin receptor.

The term “monovalent binding mode” denotes a specific binding to the BBBR where the interaction between the monovalent binding entity and the BBBR takes place through one single epitope. The monovalent binding mode prevents any dimerization/multimerization of the BBBR due to a single epitope interaction point. The monovalent binding mode prevents that the intracellular sorting of the BBBR is altered.

The term “epitope” denotes any polypeptide determinant capable of specific binding to an antibody. In certain embodiments, epitope determinants include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The “transferrin receptor” (TfR) is a transmembrane glycoprotein (with a molecular weight of about 180,000 Da) which is composed of two disulfide-bonded sub-units (each of apparent molecular weight of about 90,000 Da) and is involved in iron uptake in vertebrates. In one embodiment, the TfR herein is human TfR comprising the amino acid sequence as reported in Schneider et al. (Nature 311 (1984) 675-678).

The term “neurological disorder” denotes a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, ShyDraeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, GerstmannStraussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroidlipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body).

The term “neurological disorder drug” denotes a drug or therapeutic agent that treats one or more neurological disorder(s). Neurological disorder drugs include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Exemplary neurological disorder drugs are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), AntiEpidermal Growth Factor Receptor Brain cancer, (EGFR)-antibody, glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

The term “imaging agent” denotes a compound that has one or more properties that permit its presence and/or location to be detected directly or indirectly. Examples of such imaging agents include proteins and small molecule compounds incorporating a labeled entity that permits detection.

The terms “CNS antigen” and “brain target” denote an antigen and/or molecule expressed in the CNS, including the brain, which can be targeted with an antibody or small molecule. Examples of such antigen and/or molecule include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In one embodiment, the antigen is BACEL.

The term “that specifically binds” denotes an antibody selectively or preferentially binding to an antigen. The binding affinity is generally determined using a standard assay, such as Scatchard analysis, or surface plasmon resonance technique (e.g. using BIACORE®).

The term “CH2-CH3 Ig entity” as used herein refers to a protein entity derived from immunoglobulin CH2 or CH3 domains. The “CH2-CH3 Ig entity” comprises two “CH2-CH3” polypeptides forming a dimer. The immunoglobulin can be IgG, IgA, IgD, IgE or IgM. In one embodiment, the CH2-CH3 Ig entity derived from an IgG immunoglobulin and is referred to herein as “CH2-CH3 IgG entity”. The term includes native sequence of CH2-CH3 domains and variant CH2-CH3 domains. In one embodiment, the “CH2-CH3 Ig entity” derives from human heavy chain CH2-CH3 IgG domain which extends from Cys226, or from Pro230, to the carboxyl-terminus of the heavy chain. However, the C-terminal lysine (Lys447) of the Fc region may or may not be present. Unless otherwise specified herein, numbering of amino acid residues in the CH2-CH3 domain region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “conjugate” is fusion protein of the present invention conjugated to one or more heterologous molecule(s), including but not limited to a label, neurological disorder drug or cytotoxic agent.

The term “linker” denotes a chemical linker or a single chain peptidic linker that covalently connects different entities of the blood brain barrier shuttle module and/or the fusion polypeptide and/or the conjugate as reported herein. The linker connects for example the brain effector entity to the monovalent binding entity. For example, if the monovalent binding entity comprises a CH2-CH3 Ig entity and a scFab directed to the blood brain barrier receptor, then the linker conjugates the scFab to the C-terminal end of the CH3-CH2 Ig entity. The linker conjugating the brain effector entity to the monovalent binding entity (first linker) and the linker connecting the scFab to the C-terminal end of the CH2-CH3 Ig domain (second linker) can be the same or different.

Single chain peptidic linkers, comprising of from one to twenty amino acid residues joined by peptide bonds, can be used. In certain embodiments, the amino acids are selected from the twenty naturally-occurring amino acids. In certain other embodiments, one or more of the amino acids are selected from glycine, alanine, proline, asparagine, glutamine and lysine. In other embodiments, the linker is a chemical linker. In certain embodiments, the linker is a single chain peptidic linker with an amino acid sequence with a length of at least 25 amino acid residues, in one preferred embodiment with a length of 32 to 50 amino acid residues. In one embodiment the peptidic linker is a (GxS)n linker with G=glycine, S=serine, (x=3, n=8, 9 or 10) or (x=4 and n=6, 7 or 8), in one embodiment with x=4, n=6 or 7, in one preferred embodiment with x=4, n=7. In one embodiment the linker is (G4S)4 (SED ID NO: 08). In one embodiment the linker is (G4S)6G2 (SEQ ID NO: 07).

Conjugation may be performed using a variety of chemical linkers. For example, the monovalent binding entity or the fusion polypeptide and the brain effector entity may be conjugated using a variety of bifunctional protein coupling agents such as Nsuccinimidyl-3-(2-pyridyldithio) propionate (SPDP), succinimidyl-4-(Nmaleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52 (1992) 127-131; U.S. Pat. No. 5,208,020) may be used.

Covalent conjugation can either be direct or via a linker. In certain embodiments, direct conjugation is by construction of a polypeptide fusion (i.e. by genetic fusion of the two genes encoding the monovalent binding entity towards the BBBR and effector entity and expressed as a single polypeptide (chain)). In certain embodiments, direct conjugation is by formation of a covalent bond between a reactive group on one of the two portions of the monovalent binding entity against the BBBR and a corresponding group or acceptor on the brain effector entity. In certain embodiments, direct conjugation is by modification (i.e. genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (i.e. an amino acid) with a desired reactive group (i.e. a cysteine residue) may be introduced into, e.g., the monovalent binding entity towards the BBBR antibody and a disulfide bond formed with the neurological drug. Methods for covalent conjugation of nucleic acids to proteins are also known in the art (i.e., photocrosslinking, see, e.g., Zatsepin et al. Russ. Chem. Rev. 74 (2005) 77-95). Conjugation may also be performed using a variety of linkers. For example, a monovalent binding entity and a effector entity may be conjugated using a variety of bifunctional protein coupling agents such as Nsuccinimidyl-3-(2-pyridyldithio) propionate (SPDP), succinimidyl-4-(Nmaleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis(p-diazoniumbenzoyl)-ethylenediamine), diisocyanates (such as toluene 2,6-diisocyanate), and bis-active fluorine compounds (such as 1,5-difluoro-2,4-dinitrobenzene). Peptidic linkers, comprised of from one to twenty amino acid residues joined by peptide bonds, may also be used. In certain such embodiments, the amino acid residues are selected from the twenty naturally-occurring amino acids. In certain other such embodiments, one or more of the amino acid residues are selected from glycine, alanine, proline, asparagine, glutamine and lysine. The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52 (1992) 127-131; U.S. Pat. No. 5,208,020) may be used.

### II. Compositions and Methods

In one aspect, the invention is based, in part, on the finding that the blood brain barrier shuttle modules as reported herein can be used to deliver a brain effector entity across the blood brain barrier into the brain. In certain embodiments, the blood brain barrier shuttle module comprises a monovalent binding entity that specifically binds to a blood brain barrier receptor, such as the transferrin receptor. The blood brain barrier shuttle modules as reported herein are useful, e.g., for the diagnosis or treatment of neurological disorders, such as Alzheimer's disease, Parkinson's Disease and Alzheimer's Disease with Parkinson's Disease co-morbidity.

### A. Exemplary Anti-Blood Brain Barrier Receptor Antibodies

Monovalent binding entities that specifically bind to a blood brain barrier receptor can be characterized with respect to their binding and transcytosis properties:
efficient cell binding of BBBR expressing cells as monovalent binding entity,
efficient in vitro transcytosis as monovalent binding entity,
human—cynomolgus cross-reactivity (e.g. in BIAcore and FACS experiments).

The transcytosis screening was performed in an hCMEC/D3 based assay. The assay was performed in a pulse-chase mode. The hCMEC/D3 brain endothelial cells were incubated with the monovalent binding entity for 1 hour, washed thereafter and the following parameters were determined 0 hours and 4 hours post washing:
i) amount of monovalent binding entity taken up into the cells during the loading phase,
ii) basolateral amount of monovalent binding entity 4 hours post loading and washing;
iii) apical amount of monovalent binding entity 4 hours post loading and washing;
iv) amount of monovalent binding entity in the cells (by cell lysis) 0 hours and 4 hours after loading and washing;
v) total amount of monovalent binding entity 0 hours and 4 hours after loading and washing.

In order to be eligible as monovalent binding entity in a blood brain barrier shuttle module as reported herein the monovalent binding entity has to be i) taken up by the hCMEC/D3 cells (endocytosis), ii) transported outside the hCMEC/D3 cells (exocytosis), and iii) stable inside the hCMEC/D3 cells (no or low transport to the endosome for degradation).

Thus, in one embodiment the monovalent binding entity is characterized in a hCMEC/D3 based assay by i) an (substantial) uptake into the hCMEC/D3 cells during a one-hour loading period, ii) a release into the apical and/or basolateral compartment after the loading period and a washing step within 4 hours after the washing, and iii) a low (intracellular) degradation rate.

In one embodiment the loading is at a concentration of about 2.67 μg/mL monovalent binding entity for one hour.

It has been found that a monovalent binding entity in order to be eligible as monovalent binding entity of a blood brain barrier shuttle module as reported herein has to show in the above described hCMEC/D3 based assay the following threshold values:
i) an amount of monovalent binding entity taken up into the cells during the loading phase of 400 μg or more,
ii) basolateral amount of monovalent binding entity 4 hours post loading and washing of 100 μg or more, and
iii) apical amount of monovalent binding entity 4 hours post loading and washing of 150 μg or more.

The mouse anti-human transferrin-receptor antibody 128.1 (for variable region sequences see WO 93/10819 and SEQ ID NO: 11 and 12) can be taken as reference. In this case the monovalent binding entity in order to be eligible as monovalent binding entity of a blood brain barrier shuttle module as reported herein has to show in the above described hCMEC/D3 based assay the following threshold values:
i) an amount of monovalent binding entity taken up into the cells during the loading phase of 20% or more of the loading of antibody 128.1,
ii) basolateral amount of monovalent binding entity 4 hours post loading and washing of 15% or more of the basolateral amount of antibody 128.1; and
iii) apical amount of monovalent binding entity 4 hours post loading and washing of 15% or more of the apical amount of antibody 128.1.

The hCMEC/D3 based assay was performed as follows (this is one embodiment of all aspects as reported herein):

Medium and supplements for hCMEC/D3 (see WO 2006/056879 and Weksler, B. B., et al., FASEB J. 19 (2005) 1872-1874) can be obtained from Lonza. hCMEC/D3 cells (passages 26-29) are/can be cultured to confluence on collagen-coated coverslips (microscopy) or flasks in EBM2 medium containing 2.5% FBS, a quarter of the supplied growth factors and fully complemented with supplied hydrocortisone, gentamycin and ascorbic acid.

For all transcytosis assays, high density pore (1×10⁸ pores/cm²) PET membrane filter inserts (0.4 μm pore size, 12 mm diameter) are/can be used in 12-well cell culture plates. Media volumes are calculated to be 400 μL and 1600 μL for apical and basolateral chambers, respectively. Apical chambers of filter inserts are/can be coated with rat tail collagen I (7.5 μg/cm²) followed by fibronectin (5 μg/mL), each incubation lasting for 1 h at RT. hCMEC/D3 cells are/can be grown to confluent monolayers (˜2×10⁵ cells/cm²) for 10-12 days in EBM2 medium. Empty filters are/can be blocked in PBS containing 1% BSA for 1 hour or overnight (o/n) before assay and then calibrated for at least 1 hour in EBM2 before the assay.

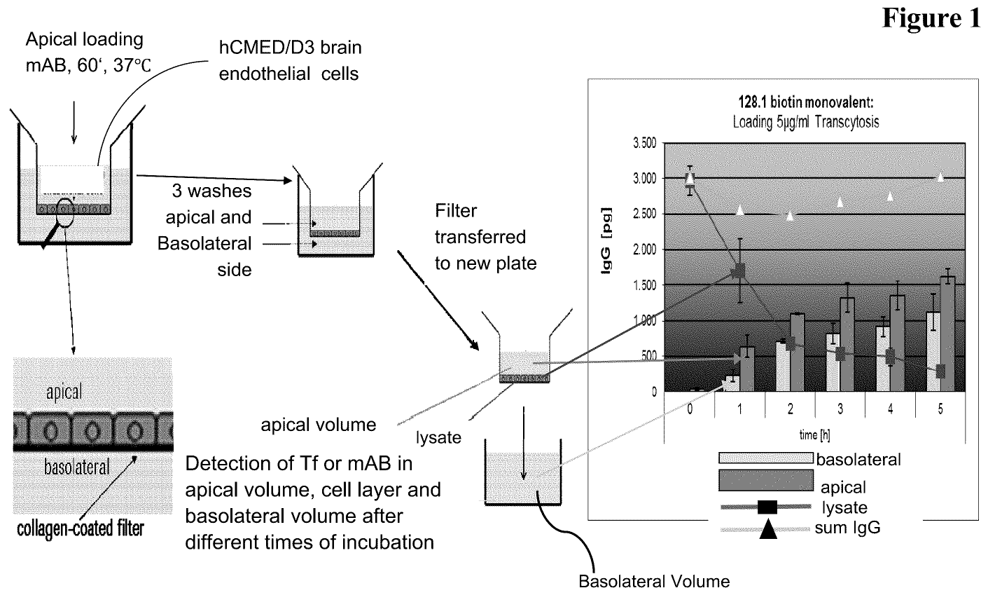

The assay (for assay scheme see FIG. 1) was performed in serum-free EBM2 medium which was otherwise reconstituted as described herein. On day of the assay, cells are serum-starved for 60 min. to deplete the natural ligand of the blood brain barrier receptor in question. Filter inserts with or without (but blocked overnight in complete medium) cells were incubated apically with monoclonal antibodies in question (monovalent binding entity) for 1 hour at 37° C. The monolayers were washed at room temperature (RT) in serum-free medium apically (400 μL) and basolaterally (1600 μL) three times for 3-5 min. each. Pre-warmed medium was added to the apical chamber and the filters transferred to a fresh 12 well plate (blocked overnight with PBS containing 1% BSA) containing 1600 μL pre-warmed medium. At this point, filters with or without cells were lysed in 500 μL RIPA buffer in order to determine specific antibody (monovalent binding entity) uptake. The remaining filters were incubated at 37° C. or at 4° C. and samples were collected at various time points to determine apical and/or basolateral release of the antibody (monovalent binding entity). The content of antibody in the samples can be quantified using a highly sensitive IgG ELISA (see Example 10). For each time point, data should be generated from two empty filters and three filter cell cultures.

The results for 69 anti-transferrin receptor antibodies are shown in the Table below. Antibody 128.1 was used as reference.

| values of | | | | | | | |
|---|---|---|---|---|---|---|---|
| reference | transcytosis | total | total | | | | |
| antibody | loading | basolateral | apical | | | | |
| 128.1 | 0 h | 4 h | 4 h | | | | |
| for set | [pg] | [pg] | [pg] | | | | |
| 1 | 2072 | 896 | 1547 | | | | |
| 2 | 2951 | 797 | 1690 | | | | |
| 3 | 3448 | 1034 | 1843 | | | | |
| 4 | 864 | 274 | 375 | | | | |
| 5 | 3027 | 957 | 1679 | | | | |
| 6 | 2133 | 638 | 1187 | | | | |
| 7 | 3490 | 1138 | 1966 | | | | |
| | transcytosis | total | total | | relative | relative | relative |
| | loading | basolateral | apical | | loading to | basolateral | apical of |
| | 0 h | 4 h | 4 h | | reference | of reference | reference |
| clone | [pg] | [pg] | [pg] | set | [%] | [%] | [%] |
| No. 0116 | 872 | 70 | 203 | 1 | 42 | 8 | 13 |
| No. 0127 | 293 | 56 | 160 | 1 | 10 | 7 | 9 |
| No. 0128 | 1155 | 231 | 746 | 3 | 33 | 22 | 40 |
| No. 0134 | 151 | 18 | 154 | 3 | 4 | 2 | 8 |
| No. 0143 | 651 | 156 | 475 | 2 | 22 | 20 | 28 |
| No. 0146 | 1623 | 357 | 748 | 2 | 55 | 45 | 44 |
| No. 0150 | 298 | 36 | 205 | 3 | 9 | 3 | 11 |
| No. 0279 | 768 | 77 | 131 | 4 | 89 | 28 | 35 |
| No. 0280 | 221 | 42 | 133 | 4 | 26 | 15 | 35 |
| No. 0281 | 34 | 3 | 16 | 4 | 4 | 1 | 4 |
| No. 0282 | 574 | 14 | 45 | 4 | 66 | 5 | 12 |
| No. 0283 | 112 | 24 | 52 | 4 | 13 | 9 | 14 |
| No. 0284 | 32 | 0 | 24 | 4 | 4 | 0 | 6 |
| No. 0285 | 21 | 0 | 18 | 4 | 2 | 0 | 5 |
| No. 0286 | 130 | 29 | 93 | 4 | 15 | 11 | 25 |
| No. 0291 | 419 | 61 | 96 | 4 | 48 | 22 | 26 |
| No. 0292 | 118 | 27 | 95 | 4 | 14 | 10 | 25 |
| No. 0293 | 354 | 28 | 88 | 4 | 41 | 10 | 23 |
| No. 0294 | 25 | 0 | 12 | 4 | 3 | 0 | 3 |
| No. 0295 | 101 | 21 | 75 | 4 | 12 | 8 | 20 |
| No. 0297 | 95 | 17 | 64 | 4 | 11 | 6 | 17 |
| No. 0298 | 195 | 10 | 59 | 4 | 23 | 4 | 16 |
| No. 0299 | 705 | 170 | 294 | 4 | 82 | 64 | 79 |
| No. 0300 | 632 | 103 | 86 | 4 | 73 | 38 | 23 |
| No. 0301 | 61 | 10 | 41 | 4 | 7 | 4 | 11 |
| No. 0302 | 420 | 118 | 217 | 4 | 49 | 43 | 58 |
| No. 0304 | 665 | 157 | 282 | 4 | 77 | 58 | 75 |
| No. 0305 | 154 | 33 | 111 | 4 | 18 | 12 | 30 |
| No. 0313 | 175 | 36 | 132 | 5 | 6 | 4 | 8 |
| No. 0316 | 84 | 0 | 72 | 5 | 3 | 0 | 4 |
| No. 0318 | 130 | 21 | 82 | 5 | 4 | 2 | 5 |
| No. 0319 | 406 | 104 | 343 | 5 | 13 | 11 | 20 |
| No. 0322 | 172 | 84 | 96 | 5 | 6 | 9 | 6 |
| No. 0323 | 794 | 175 | 478 | 5 | 26 | 18 | 28 |
| No. 0324 | 193 | 32 | 144 | 5 | 6 | 3 | 9 |
| No. 0325 | 664 | 166 | 483 | 5 | 22 | 17 | 29 |
| No. 0329 | 744 | 192 | 449 | 6 | 35 | 30 | 38 |
| No. 0335 | 601 | 0 | 45 | 6 | 28 | 0 | 4 |
| No. 0339 | 967 | 227 | 580 | 6 | 45 | 36 | 49 |
| No. 0343 | 883 | 195 | 574 | 6 | 41 | 34 | 48 |
| No. 0346 | 1684 | 424 | 832 | 6 | 79 | 67 | 70 |
| No. 0348 | 838 | 205 | 626 | 6 | 39 | 32 | 53 |
| No. 0349 | 1538 | 181 | 396 | 6 | 72 | 28 | 33 |
| No. 0350 | 940 | 257 | 637 | 6 | 44 | 40 | 54 |
| No. 0411 | 1655 | 512 | 955 | 6 | 78 | 80 | 80 |
| No. 0431 | 1738 | 511 | 493 | 6 | 81 | 80 | 42 |
| No. 0432 | 890 | 265 | 421 | 6 | 42 | 41 | 35 |
| No. 0433 | 957 | 274 | 370 | 6 | 45 | 43 | 31 |
| No. 0434 | 1033 | 264 | 403 | 6 | 48 | 41 | 34 |
| No. 0435 | 1356 | 351 | 503 | 6 | 64 | 55 | 42 |
| No. 0441 | 1256 | 158 | 383 | 6 | 59 | 25 | 32 |
| No. 0445 | 758 | 133 | 196 | 6 | 36 | 21 | 17 |
| No. 0447 | 451 | 133 | 320 | 6 | 21 | 21 | 18 |
| No. 0449 | 728 | 214 | 377 | 6 | 34 | 34 | 32 |
| No. 0451 | 797 | 174 | 442 | 6 | 37 | 27 | 37 |
| No. 0452 | 822 | 291 | 490 | 6 | 39 | 46 | 41 |
| No. 0453 | 720 | 220 | 500 | 6 | 34 | 34 | 42 |
| No. 0486 | 1434 | 454 | 503 | 7 | 41 | 40 | 26 |
| No. 0487 | 4583 | 685 | 1364 | 7 | 131 | 60 | 69 |
| No. 0488 | 991 | 176 | 658 | 7 | 28 | 15 | 33 |
| No. 0489 | 2124 | 370 | 1179 | 7 | 61 | 33 | 60 |
| No. 0490 | 4011 | 581 | 1388 | 7 | 115 | 51 | 71 |
| No. 0491 | 1310 | 244 | 762 | 7 | 38 | 21 | 39 |
| No. 0492 | 2962 | 516 | 1052 | 7 | 85 | 45 | 54 |
| No. 0493 | 1006 | 215 | 885 | 7 | 29 | 19 | 45 |
| No. 0494 | 3510 | 748 | 1503 | 7 | 101 | 66 | 76 |
| No. 0495 | 1236 | 184 | 617 | 7 | 35 | 16 | 31 |
| No. 0496 | 2178 | 539 | 1402 | 7 | 64 | 47 | 71 |
| No. 0497 | 2984 | 725 | 1809 | 7 | 85 | 64 | 92 |

Antibody 299 shows a transcytosis loading of 705 μg, whereof after 4 hours 170 μg (24% of loading) can be found in the basolateral compartment and 294 μg (=42% of loading) can be found in the apical compartment.

Antibody 494 shows a transcytosis loading of 3510 μg, whereof after 4 hours 748 μg (=21% of loading) can be found in the basolateral compartment and 1503 μg (=43% of loading) can be found in the apical compartment.

The antibodies fulfilling the criteria as outlined above are embodiments of the current invention.

Thus, one aspect as reported herein is an anti-transferrin receptor antibody or transferrin receptor binding fragment thereof comprising
(1) a heavy chain variable domain that has the amino acid sequence of SEQ ID NO: 13 and a light chain variable domain that has the amino acid sequence of SEQ ID NO: 14, or
(2) a heavy chain variable domain that has the amino acid sequence of SEQ ID NO: 15 and a light chain variable domain that has the amino acid sequence of SEQ ID NO: 16, or
(3) a heavy chain variable domain that has the amino acid sequence of SEQ ID NO: 17 and a light chain variable domain that has the amino acid sequence of SEQ ID NO: 18, or
(4) a heavy chain variable domain that has the amino acid sequence of SEQ ID NO: 19 and a light chain variable domain that has the amino acid sequence of SEQ ID NO: 20, or
(5) a heavy chain variable domain that has the amino acid sequence of SEQ ID NO: 21 and a light chain variable domain that has the amino acid sequence of SEQ ID NO: 22, or
(6) a heavy chain variable domain that has the amino acid sequence of SEQ ID NO: 23 and a light chain variable domain that has the amino acid sequence of SEQ ID NO: 24, or
(7) a heavy chain variable domain that has the amino acid sequence of SEQ ID NO: 25 and a light chain variable domain that has the amino acid sequence of SEQ ID NO: 26, or
(8) a heavy chain variable domain that has the amino acid sequence of SEQ ID NO: 27 and a light chain variable domain that has the amino acid sequence of SEQ ID NO: 28, or
(9) a heavy chain variable domain that has the amino acid sequence of SEQ ID NO: 29 and a light chain variable domain that has the amino acid sequence of SEQ ID NO: 30, or
(10) a heavy chain variable domain that has the amino acid sequence of SEQ ID NO: 31 and a light chain variable domain that has the amino acid sequence of SEQ ID NO: 32, or
(11) a heavy chain variable domain that has the amino acid sequence of SEQ ID NO: 33 and a light chain variable domain that has the amino acid sequence of SEQ ID NO: 34, or
(12) a heavy chain variable domain that has the amino acid sequence of SEQ ID NO: 35 and a light chain variable domain that has the amino acid sequence of SEQ ID NO: 36, or
(13) a heavy chain variable domain that has the amino acid sequence of SEQ ID NO: 37 and a light chain variable domain that has the amino acid sequence of SEQ ID NO: 38, or
(14) a heavy chain variable domain that has the amino acid sequence of SEQ ID NO: 39 and a light chain variable domain that has the amino acid sequence of SEQ ID NO: 40, or
(15) a heavy chain variable domain that has the amino acid sequence of SEQ ID NO: 41 and a light chain variable domain that has the amino acid sequence of SEQ ID NO: 42, or
(16) a heavy chain variable domain that has the amino acid sequence of SEQ ID NO: 43 and a light chain variable domain that has the amino acid sequence of SEQ ID NO: 44, or
(17) a heavy chain variable domain that has the amino acid sequence of SEQ ID NO: 45 and a light chain variable domain that has the amino acid sequence of SEQ ID NO: 46, or
(18) a heavy chain variable domain that has the amino acid sequence of SEQ ID NO: 47 and a light chain variable domain that has the amino acid sequence of SEQ ID NO: 48, or
(19) a heavy chain variable domain that has the amino acid sequence of SEQ ID NO: 49 and a light chain variable domain that has the amino acid sequence of SEQ ID NO: 50, or
(20) a heavy chain variable domain that has the amino acid sequence of SEQ ID NO: 51 and a light chain variable domain that has the amino acid sequence of SEQ ID NO: 52, or
(21) a heavy chain variable domain that has the amino acid sequence of SEQ ID NO: 53 and a light chain variable domain that has the amino acid sequence of SEQ ID NO: 54, or
(22) a heavy chain variable domain that has the amino acid sequence of SEQ ID NO: 55 and a light chain variable domain that has the amino acid sequence of SEQ ID NO: 56, or
(23) a heavy chain variable domain that has the amino acid sequence of SEQ ID NO: 57 and a light chain variable domain that has the amino acid sequence of SEQ ID NO: 58, or
(24) a heavy chain variable domain that has the amino acid sequence of SEQ ID NO: 59 and a light chain variable domain that has the amino acid sequence of SEQ ID NO: 60, or
(25) a heavy chain variable domain that has the amino acid sequence of SEQ ID NO: 61 and a light chain variable domain that has the amino acid sequence of SEQ ID NO: 62, or
(26) a heavy chain variable domain that has the amino acid sequence of SEQ ID NO: 63 and a light chain variable domain that has the amino acid sequence of SEQ ID NO: 64, or
(27) a heavy chain variable domain that has the amino acid sequence of SEQ ID NO: 65 and a light chain variable domain that has the amino acid sequence of SEQ ID NO: 66, or
(28) a heavy chain variable domain that has the amino acid sequence of SEQ ID NO: 67 and a light chain variable domain that has the amino acid sequence of SEQ ID NO: 68, or
(29) a heavy chain variable domain that has the amino acid sequence of SEQ ID NO: 69 and a light chain variable domain that has the amino acid sequence of SEQ ID NO: 70, or
(30) a heavy chain variable domain that has the amino acid sequence of SEQ ID NO: 71 and a light chain variable domain that has the amino acid sequence of SEQ ID NO: 72, or
(31) a heavy chain variable domain that has the amino acid sequence of SEQ ID NO: 73 and a light chain variable domain that has the amino acid sequence of SEQ ID NO: 74, or
(32) a heavy chain variable domain that has the amino acid sequence of SEQ ID NO: 75 and a light chain variable domain that has the amino acid sequence of SEQ ID NO: 76, or
(33) a heavy chain variable domain that has the amino acid sequence of SEQ ID NO: 77 and a light chain variable domain that has the amino acid sequence of SEQ ID NO: 78, or
(34) a heavy chain variable domain that has the amino acid sequence of SEQ ID NO: 79 and a light chain variable domain that has the amino acid sequence of SEQ ID NO: 80, or
(35) a heavy chain variable domain that has the amino acid sequence of SEQ ID NO: 81 and a light chain variable domain that has the amino acid sequence of SEQ ID NO: 82, or
(36) a heavy chain variable domain that has the amino acid sequence of SEQ ID NO: 83 and a light chain variable domain that has the amino acid sequence of SEQ ID NO: 84, or
(37) a heavy chain variable domain that has the amino acid sequence of SEQ ID NO: 85 and a light chain variable domain that has the amino acid sequence of SEQ ID NO: 86, or
(38) a heavy chain variable domain that has the amino acid sequence of SEQ ID NO: 87 and a light chain variable domain that has the amino acid sequence of SEQ ID NO: 88, or
(39) a humanized heavy chain variable domain derived from the heavy chain variable domain that has the amino acid sequence of SEQ ID NO: 89 and a light chain variable domain that has the amino acid sequence of SEQ ID NO: 90, or
(40) a heavy chain variable domain that has the amino acid sequence of SEQ ID NO: 91 and a light chain variable domain that has the amino acid sequence of SEQ ID NO: 92.

(41) a heavy chain variable domain that has the amino acid sequence of SEQ ID NO: 93 and a light chain variable domain that has the amino acid sequence of SEQ ID NO: 94, or
(42) a heavy chain variable domain that has the amino acid sequence of SEQ ID NO: 95 and a light chain variable domain that has the amino acid sequence of SEQ ID NO: 96, or
(43) a heavy chain variable domain that has the amino acid sequence of SEQ ID NO: 97 and a light chain variable domain that has the amino acid sequence of SEQ ID NO: 98.

One preferred aspect as reported herein is an anti-transferrin receptor antibody or transferrin receptor binding fragment thereof comprising a heavy chain variable domain that has the amino acid sequence of SEQ ID NO: 23 and a light chain variable domain that has the amino acid sequence of SEQ ID NO: 24.

One preferred aspect as reported herein is an anti-transferrin receptor antibody or transferrin receptor binding fragment thereof comprising a heavy chain variable domain that has the amino acid sequence of SEQ ID NO: 91 and a light chain variable domain that has the amino acid sequence of SEQ ID NO: 92.

The respective amino acid sequences are depicted in the following Table.

| | heavy chain variable domain | light chain variable domain |
|---|---|---|
| clone | amino acid sequence (SEQ ID NO:) | amino acid sequence (SEQ ID NO:) |
| No. 0128 | QSVEESGGRLVTPGTPLTLTCTVSGIDLS | DIQMTQSPSSLSASVGDRVTITCRASQSI |
| | SYYMSWVRQAPGKGLEWIGII | SNYLNWYQQKPGKAPKLLIYD |
| | YPSGYAYYTSWAKGRFTISKTSTTVDLK | ASSLESGVPSRFSGSGSGTDFTLTISSLQP |
| | MTSLTTEDTATYFCARDVGDGG | EDFATYYCQQSGSTPYTFGQ |
| | GTFDIWGQGTMVTVSS (13) | GTKLEIKR (14) |
| No. 0143 | GVQCQSLEESGGRLVTPGGSLTLTCTVS | DIQMTQSPSSLSVSVGDRVTITCRASQGI |
| | GIDLSTYSMGWVRQSPGKGLEY | SNYLAWYQQKPGKAPELLIYA |
| | IGIISSSDNTYYTSWAKGRFTISKTSTTVD | ASILQGGVPSRFSGSGSGTDFTLTISSLQ |
| | LKITSPTTEDTATYFCARGY | PEDVATYYCQQYNTYPPTFGQ |
| | NGGWKTPMDVWGQRTTVTVSS (15) | GTKLEIKR (16) |
| No. 0146 | GVQCEVQLVESGGGLVQPGGSLRLSCA | DIQMTQSPSSLSASVGDRVTITCRASQSI |
| | ASGFTFGDFAMNWVRQAPGKGLE | SSYLNWYQQKPGKAPKLLIYS |
| | WVSGISASGVSTYYADSVKGRVTISRDN | ASSLQSGVPSRFSGSRSGTEFTLTISSLQP |
| | SKNTLNLQMNSLRAEDTAVYYC | EDFATYYCQQSYNTPLTFGG |
| | ARDPCDASNDYGYCKLDVWGQGTTVT | GTKVEIKR (18) |
| | VSS (17) | |
| No. 0279 | QSVEESGGRLVTPGTPLTLTCTVSGIDLS | AYDMTQTPASVEVAVGGTVTIKCQASQ |
| | SYAMGWVRQAPGKGLEWIGYI | SISNYLAWYQQKPGQPPKLLIYR |
| | WSGGSADYASWAKGRFTISKTSTTVDL | ASTLASGVSSRFKGSGSGTDFTLTISGV |
| | KIPSPTMEDTATYFCARGDAEYG | GCADAATYYCQQTLSTINIDNT |
| | DANGFAIWGPGTLVTVSL (19) | FGGGTEVVVKR (20) |
| No. 0291 | QSVEESGGRLVTPGTPLTLTCTVSGFSLS | AYDMTQTPASVEVAVGGTVTIKCQASQ |
| | SGAMTWVRQAPGKGLEWIGYI | SISSYLAWYQQKPGQPPKLLIYR |
| | WSGGSTDYASWAKGRFTISKTSTTVDLK | ASTLASGVSSRFKGSGSGTQFTLTISDLE |
| | ITSPTTEDTATYFCARGYGADY | CADAATYYCQQGLSTINVDNT |
| | PDFGDANGFDPWGPGTLVTVSS (21) | FGGGTEVVVKR (22) |
| No. 0299 | QSMEESGGRLVTPGTPLTLTCTVSGFSLS | AYDMTQTPASVEVAVGGTVTIKCQASQ |
| | SYAMSWVRQAPGKGLEWIGYI | SISSYLSWYQQKPGQRPKLLIYR |
| | WSGGSTDYASWAKGRFTISKTSTTVDLK | ASTLASGVSSRFKGSGSGTQFTLTISGVE |
| | ITSPTTEDTATYFCARRYGTSY | CADAATYYCQQCYSSSNVDNT |
| | PDYGDANGFDPWGPGTLVTVSS (23) | FGGGTEVVVKR (24) |
| No. 0300 | QSVEESGGRLVTPGTPLTLTCTASEFSLS | ADVVMTQTPASVEAAVGGTVTIKCQAS |
| | NYYMSWVRQAPGKGLEYIGFI | QSISSYFAWYQQKPGQPPKLLIY |
| | HTDGSTNYASWVNGRFTISRTSTTVDLK | RASKLATGVPSRFKGSGSGTEFTLTISDL |
| | TTSLTTEDTATYFCCRYLVSGG | ECADAATYYCQTCGSISTYGG |
| | TIAFDLWGPGTLVTVSS (25) | AFGGGTEVVVKR (26) |
| No. 0302 | QSLEESGGRLVTPGGSLTLTCTVSGFSLS | AFEMTQTPSSVSEPVGGTVTIKCQASEN |
| | TYAMTWVRQAPGKGLEWIGII | IYSFLAWYQQKPGQPPKLLIYD |
| | YAGTDITWYASWAKGRFTISKTSTTVDL | ASDLASGVPSRFKGSGSGTQFTLTISDLE |
| | SITSPTTEDTATYFCAKVPYTG | CADAATYYCQQGYSGNVILNA |
| | VSDYLYYFDLWGPGTLVTVSS (27) | FGGGTEVVVKR (28) |
| No. 0304 | QSMEESGGRLVTPGTPLTLTCTVSGFSLS | AYDMTQTPASVEVAVGGTVTIKCQASQ |
| | SYAMSWVRQAPGKGLEWIGYI | SISSYLSWYQQKPGQRPKLLIYR |
| | WSGGSTDYASWAKGRFTISKTSTTVDLK | ASTLASGVSSRFKGSGSGTQFTLTISGVE |
| | ITSPTTEDTATYFCARRYGTSY | CADAATYYCQQCYSSSNVDNT |
| | PDYGDANGFDPWGPGTLVTVSS (29) | FGGGTEVVVKR (30) |
| No. 0323 | QSVEESGGRLVTPGTPLPLTCTVSGFSLS | AYDMTQTPASVEVAVGGTVTIKCQASQ |
| | NYAMGWFRQAPGKGLEWIGYI | SISIYLAWYQQKPGQPPKLLIYK |
| | WSGGSTDYASWAKGRFTISKTSTTVDLK | ASTLASGVPSRFKGSGSGTEFTLTISDLE |
| | MTSLTTEDTATYFCARADTVYR | CADAATYYCQQAYSYSNVDNV |
| | DDYGWSLWGPGTLVTVSS (31) | FGGGTEVVVKR (32) |
| No. 0325 | QSVEESGGRLVTPGTPLTLTCTVSGFSLS | AYDMTQTPASVEVAVGGTVTIKCQASE |
| | NYNMGWVRQAPGKGLEWIGII | DIESYLAWYQQKPGQRPKLLIYA |
| | SDAGSAWYASWVKGRFTISKTSTTVDL | VSTLASGVSSRFKGSGSGTEYTLTISGV |
| | KITSPTTEDAATYFCARGDAAAY | QCADAATYYCQQGYSSSNLDNA |
| | TTGTEFFSLWGPGTLVTVSS (33) | FGGGTEVVVRR (34) |
| No. 0329 | GVQCQSLEESGGGLVKPGGTLTLTCTVS | AYDMTQTPASVEAAVGGTVTIKCQASQ |
| | GFSLNSYSMTWVRQAPGKGLEW | SITSYLAWYQQKPGQPPKLLIYR |
| | IGYIWSGGSADYANWAKGRFTISKTSTT | ASTLASGVSSRFKGSGSGTQFTLTISGVE |
| | VDLKMTSLTAEDTATYFCGRGY | CADAATYYCQQTYRYMNVDNV |
| | DTTDNGFNLWGPGTLVTVSS (35) | FGGGTEVVVKR (36) |
| No. 0339 | GVQCQSVEESGGRLVTPGTPLTLTCTVS | AYDMTQTPSSVEAAVGGTVTIKCQASQ |
| | GIDLSSHAMTWVRQAPGKGLEW | SISNYLAWYQQKPGQPPELLIYR |
| | IGYIWSGGSADYASWAKGRFTISKTSSTT | ASTLASGVSSRFRGSGSGTDFTLTISGVG |
| | VDLKIPSPTTEDTATYFCARG | CADAATYYCQQGLSSSYVDNT |
| | ADEYGDANGFNIWGPGTLVTVSL (37) | FGGGTEVVVKR (38) |
| No. 0343 | GVQCQEQLKESGGGLVTPGTPLTLTCTA | ADVVMTQTPSPVSGAVGGTVTIKCQAS |
| | SGFSLSIYYMSWVRQAPGKGLD | QSIDSYLSWYQQKPGQPPKLLIY |
| | WIGFIYVDSSSAHYASWAKGRFTISKTST | SASTLASGVSSRFKGSGSGTQFTLTISDL |
| | TVDLKITSPTTEDTATYFCAR | ECADAATYYCQCTYYDSSSIG |
| | DVDSSYFWGFNLWGPGTLVTVSS (39) | GFGGGTEVVVKR (40) |
| No. 0346 | GVQCQSVEESGGRLVTPGTPLTLTCTVS | DVVMTQTPSSVSEPVGGTVTINCQASEN |
| | GFSLSSNAINWVRQAPGKGLEW | IYSSLDWYQQKPGQPPKLLIYS |
| | IGYIYTDGNTYYASWAKGRFTISKTSTT | ASNLASGVSSRFKGSRSGTEYTLTISDLE |
| | VDLKITSPTTEDTATYFCARGV | CADAATYYCQCIDGGNRGKPF |
| | GYSDLWGPGTLVTVSS (41) | GGGTEVVVKR (42) |
| No. 0348 | GVQCQSVEESGGRLVTPGTPLTLTCTAS | AQVLTQTASSVSAAVGGTVTISCQSSQS |
| | GFSLNSVYMSWVRQAPGKGLEW | VWNNNFLSWYQQKPGQPPKLLI |
| | IGIIYASGSIYYASWAKGRFTISRTSSTTV | YLASTLASGVPSRFKGSGSGTQFTLTISD |
| | DLKMTSLTTEDTATYFCVRS | LECDDAATYYCAGVYSDNIFA |
| | ADYDSGMPFDLWGPGTLVTVSS (43) | FGGGTEVVVKR (44) |
| No. 0349 | GVQCQSVEESGGRLVTPGTPLTLTCTVS | ADVVMTQTPASVEAAVGGTVTIKCQAS |
| | GFSLSSSYMAWVRQAPGKGLEY | QSISSYFAWYQQKPGQPPKLLIY |
| | IGFIHTDGSTYYATWVNGRFTISRTSTTV | RASNLATGVPSRFKGSGSGTEFTLTISDL |
| | TLKMTSLTTEDTATYFCARYL | ECADAATYYCQSCGSISTYGG |
| | VGSGAVAFDLWGPGTLVTVSS (45) | AFGGGTEVVVKR (46) |
| No. 0350 | GVQCQSVEESGGRLVTPGTPLTLTCTVS | AYDMTQTPASVSEPVGGTVTIKCQASQ |
| | GFSLSSYAMGWVRQAPGKGLEW | SISSYLSWYQQKPGQPPKLLIYR |
| | IGYIWSGGSTDYATWAKGRFTISKTSTT | ASTLESGVPSRFKGSGSGTEFTLTISDLE |
| | VDLSITSPTTEDTAAYFCVRSA | CADAATYYCQQGLSVIHVDNT |
| | GSGDAMGFNLWGPGTLVTVSS (47) | FGGGTEVVVKR (48) |
| No. 0411 | QSVEESGGRLVTPGTPLTLTCTVSGFSLS | AYDMTQTPASVSAAVGGTVSINCQASQ |
| | NYAMTWVRQAPGKGLEWIGYI | SISGYLSWYQQKPGQRPKLLIYR |
| | WSGGSTDYATWAKGRFTISKTSTTVDL | ASTLASGVSSRFKGSGSGTQFTLTISGVE |
| | KITSPTTEDTATYFCARGYGAGY | CADAATYYCQQGYSIINVDNT |
| | PDYGDANGFDPWGPGTLVTVSS (49) | FGGGTEVVVKR (50) |
| No. 0431 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCAREE | EDEADYYCNSRDDYGDGVFGG |
| | SSYISGFDYWGQGTLVTVSS (51) | GTKLTVL (52) |
| No. 0432 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCAREE | EDEADYYCNSMTESMNYVFGG |
| | SWWLSGLDYWGQGTLVTVSS (53) | GTKLTVL (54) |
| No. 0433 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCAREE | EDEADYYCNSIDSSGNHDVFG |
| | STWFSGFDYWGQGTLVTVSS (55) | GGTKLTVL (56) |
| No. 0434 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCAREY | EDEADYYCNSPDSIAYGVVFG |
| | PWYIWSYDYWGQGTLVTVSS (57) | GGTKLTVL (58) |
| No. 0435 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCARES | EDEADYYCNSEDSESNLVFGG |
| | YFLSGFDYWGQGTIVTVSS (59) | GTKLTVL (60) |
| No. 0441 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCARSW | EDEADYYCNSIDSALDHVFGG |
| | FGEWMDYWGQGTLVTVSS (61) | GTKLTVL (62) |
| No. 0445 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCARDS | EDEADYYCNSRDMSLNVVFGG |
| | WYGWAFDYWGQGTLVTVSS (63) | GTKLTVL (64) |
| No. 0447 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCARES | EDEADYYCNSRDSTLIVVVFG |
| | FYGWAFDYWGQGTLVTVSS (65) | GGTKLTVL (66) |
| No. 0449 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCARET | EDEADYYCNSPDSSVNYMVFG |
| | WGWVAFDYWGQGTLVTVSS (67) | GGTKLTVL (68) |
| No. 0451 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASITITGAQA |
| | VYMELSSLRSEDTAVYYCAREE | EDEADYYCNSRDIIGDAVFGG |
| | YYYLSGFDYWGQGTLVTVSS (69) | GTKLTVL (70) |
| No. 0452 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCAREA | EDEADYYCNSIDSEGNDIVFG |
| | WDYLSSMDYWGQGTLVTVSS (71) | GGTKLTVL (72) |
| No. 0453 | QVQLVQSGAEVKKPGASVKVSCKASGY | SSELTQDPAVSVALGQTVRITCQGDSLR |
| | TFTSYYMHWVRQAPGQGLEWMGI | SYYASWYQQKPGQAPVLVIYGK |
| | INPSGGSTSYAQKFQGRVTMTRDTSTST | NNRPSGIPDRFSGSSSGNTASLTITGAQA |
| | VYMELSSLRSEDTAVYYCARDV | EDEADYYCNSIDLSMDIVFGG |
| | YGHLDYWGQGTLVTVSS (73) | GTKLTVL (74) |
| No. 0486 | EVQLQQSGPELEKPGASVKISCKASGYS | DVLMTQTPLSLPVSLGDQASISCRSTQS |
| | FTGYNMNWVKQSNGERLEWIGS | VVHSDGITHLEWYLQKPGQSPK |
| | IDPFYGGTTYNQKFKDKATLTVDKPSST | LLIYKVFNRFYGVPDRFSGSGSGTDFTL |
| | AYMQLTSLTAEDSAVYYCAREG | KISRVEAEDLGVYYCFQGSHVP |
| | GNYYGPWFAYWGQGTLVTVSA (75) | WTFGGGTKLEIKR (76) |
| No. 0487 | DVQLVESGGGLVQPGGSRKLSCAASGFT | DVVMTQTPLTLSVTIGQPASISCKSSQSL |
| | FSSFGMHWVRQAPEKGLEWVAY | LYTIGKTYLNWLLQRPGQSPK |
| | ISSGSGTIYYADTVKGRFTISRDNPKNTL | RLIYLVSKLDSGVPDRFSGSGSGTDFTL |
| | FLQMTTLRSEDTAMYYCARSY | KISRVEAEDLGVYYCFQSTHFP |
| | YSDFGHAMDYWGQGTSVTVSS (77) | LTFGAGTKLELRR (78) |
| No. 0488 | DVQLVESGGGLVQPGGSRKLSCAASGFT | DIQMTQSPASLSATVGETVTITCRTSGNI |
| | FSTFGMHWVRQTPERGLEWVAY | HNYLAWYQQKQGKSPQLLVYN |
| | ISSGGTNIYYADPVKGRFTISRDNPKKTL | GQTLAEGVPSKFSGSGSGTQYSLKINSL |
| | FLQMTGLRSEDTAIYYCARDG | QPEDFGSYFCHQYFYFPLTFGT |
| | FGNYWFAYWGQGTLVTVSA (79) | GTKLEIKR (80) |
| No. 0489 | EVQLQQSGTELVRPGALVRLSCKASDFN | DIVMTQSHKFMSTSVGDRVTFTCKASQ |
| | IKDYYLHWVKQRPEQGLEWVGW | DVRSAVAWYQQKAGQSPKLLIYS |
| | IDPETLNTIYGPKFQGKASITADTSSNTA | ASYRYTGVPDRFTGSGSGTDFTFTISSV |
| | YLQLSGLTSEDIAVYYCTSST | QAEDLSVYYCQQHYTTPLTFGA |
| | VISYWHFDVWGAGTSVTVSS (81) | GTKLELKR (82) |
| No. 0490 | DVQLVESGGGLVQPGGSRKLSCAASGFT | DVVMTQTPLTLSVTIGQPASISCKSSQSL |
| | FSSFGMHWVRQAPEKGLEWVAY | LYTIGKTYLNWLLQRPGQSPK |
| | ISSGSGTIYYADTMKGRFTISRDNPKNTL | RLIYLVSKLDSGVPDRFSGSGSGTDFTL |
| | FLQMTTLRSEDTAMYYCARSY | KISRVEAEDLGVYYCFQSTHFP |
| | YSDFGHAMDYWGQGTSVTVSS (83) | LTFGAGTKLELRR (84) |
| No. 0491 | DVQLVESGGGLVQPGGSRKLSCAASGFT | DIQMTQSPASLSATVGETVTITCRTSGNI |
| | FSTFGMHWVRQTPERGLEWVAY | HNYLAWYQQKQGKSPQLLVYN |
| | ISSGSTNIYYADPVKGRFTISRDNPKKTL | GQTLAEGVPSKFSGSGSGTQYSLKINSL |
| | FLQMTGLRSEDTAMYYCARDG | QPEDFGSYFCHQYFYFPLTFGT |
| | FGNYWFAYWGQGTLVTVSA (85) | GTKLEIKR (86) |
| No. 0492 | DVQLVESGGGLVQPGGSRKLSCAASGFT | DVVMTQTPLTLSVTIGQPASISCKSSQSL |
| | FSSFGMHWVRQAPEKGLEWVAY | LYTQGKTYLNWLLQRPGQSPK |
| | ISSGSSTIYYADTVKGRFTISRDNPKNTLF | RLIYLVSKLDSGVPDRFSGSGSGTDFTL |
| | LQMASLRSEDTAMYYCARSY | KISRVEAEDLGVYYCLQSTHFP |
| | YGNFGFAMDYWGQGTSVTVSS (87) | LTFGAGTKLELKR (88) |
| No. 0493 | DVQLVESGGGLVQPGGSRKLSCAASGFT | DIQMTQSPASLSATVGETVTITCRTSGNI |
| | FSTFGMHWVRQTPERGLEWVAY | HNYLAWYQQKQGKSPQLLVYN |
| | ISSDSSNIYYADPVKGRFTISRDNPKKTLF | GQTLAEGVPSKFSGSGSGTQYSLKINSL |
| | LQMTGLRSEDTAIYYCARDG | QPEDFGSYFCHQYFYFPLTFGT |
| | FGNYWFAYWGQGTLVTVSA (89) | GTKLEIKR (90) |
| No. 0494 | EVQLQQSGAVLVKPGASVKLSCPASGFN | KIVMTQSPKSMSMSVGERVTLNCRASE |
| | IKDTYIHWVIQRPEQGLEWIGR | SVDTYVSWYQQKPEQSPELLIYG |
| | IDPANGDTKCDPKFQVKATITADTSSNT | ASNRYTGVPDRFTGSGSATDFTLTISSV |
| | AYLQLSSLTSEDTAVYFCVRDY | QAEDLADYYCGQTYNYPLTFGA |
| | LYPYYFDFWGQGTTLTVSS (91) | GTKLELKR (92) |
| No. 0495 | EVQLQQSGPELVKPGASVKMSCRASGY | QIVLTQSPAIMSASPGEKVTLTCSANSSI |
| | SFPSYVVHWVKQKPGQGLEWIGY | RNMHWYQQKTGTSPKRWIYDT |
| | INPYTDGTEYNEKFKGKATLTSDKSSST | SNLASGVPSRFSGSGSGTSYSLTISSMEA |
| | AYMELSSLTSEDSAVYYCARGF | EDAATYYCHQRSSFPYTFGGG |
| | YYYSMDYWGQGTSVTVSS (93) | TKLEIKR (94) |
| No. 0496 | QVQLIQSGAELVRPGASVNLSCKASGYT | DIQMTQSSSSFSVSLGDRVTVTCKTTEDI |
| | FTDYYINWVKQRPGQGLEWIAR | YNRLAWYQHKPGNAPRLLISG |
| | IYPGSDITYYNEKFKGKATLTAEKSSSTA | ATGLETGVPSRFSGSGSGKDYTLTITSL |
| | YMQLSSLTSEDSAVYFCARSP | QTEDVATYYCLQYWSTPYTFGG |
| | PHYVGNRYYALDYWGQGTSVTVSS (95) | GTKLEIKR (96) |
| No. 0497 | QVQLQQPGAEFVKPGASVKMSCKASGY | DIKMTQSPSSMYASLGERVTITCKASQD |
| | TFTSYWITWVKQRPGQGLEWIGD | INSYLNWFQQKPGKSPKTLIYR |
| | IYPGSGSNKYNEKFKSKATLTVDTSSSTA | ANRLVDGVPSRFSGSGSGQDYSLTISSL |
| | YMHLSSLTSEDSAVYYCARER | DYEDMGIYYCLQYDEFPYTFGG |
| | PTYYGSSAWFDYWGQGTLVTVSA (97) | GTKLEIKR (98) |

One aspect as reported herein is a humanized anti-transferrin receptor antibody or transferrin receptor binding fragment thereof comprising
(1) a humanized heavy chain variable domain derived from the heavy chain variable domain that has the amino acid sequence of SEQ ID NO: 13 and a humanized light chain variable domain derived from the light chain variable domain that has the amino acid sequence of SEQ ID NO: 14, or
(2) a humanized heavy chain variable domain derived from the heavy chain variable domain that has the amino acid sequence of SEQ ID NO: 15 and a humanized light chain variable domain derived from the light chain variable domain that has the amino acid sequence of SEQ ID NO: 16, or
(3) a humanized heavy chain variable domain derived from the heavy chain variable domain that has the amino acid sequence of SEQ ID NO: 17 and a humanized light chain variable domain derived from the light chain variable domain that has the amino acid sequence of SEQ ID NO: 18, or
(4) a humanized heavy chain variable domain derived from the heavy chain variable domain that has the amino acid sequence of SEQ ID NO: 19 and a humanized light chain variable domain derived from the light chain variable domain that has the amino acid sequence of SEQ ID NO: 20, or
(5) a humanized heavy chain variable domain derived from the heavy chain variable domain that has the amino acid sequence of SEQ ID NO: 21 and a humanized light chain variable domain derived from the light chain variable domain that has the amino acid sequence of SEQ ID NO: 22, or
(6) a humanized heavy chain variable domain derived from the heavy chain variable domain that has the amino acid sequence of SEQ ID NO: 23 and a humanized light chain variable domain derived from the light chain variable domain that has the amino acid sequence of SEQ ID NO: 24, or
(7) a humanized heavy chain variable domain derived from the heavy chain variable domain that has the amino acid sequence of SEQ ID NO: 25 and a humanized light chain variable domain derived from the light chain variable domain that has the amino acid sequence of SEQ ID NO: 26, or
(8) a humanized heavy chain variable domain derived from the heavy chain variable domain that has the amino acid sequence of SEQ ID NO: 27 and a humanized light chain variable domain derived from the light chain variable domain that has the amino acid sequence of SEQ ID NO: 28, or
(9) a humanized heavy chain variable domain derived from the heavy chain variable domain that has the amino acid sequence of SEQ ID NO: 29 and a humanized light chain variable domain derived from the light chain variable domain that has the amino acid sequence of SEQ ID NO: 30, or
(10) a humanized heavy chain variable domain derived from the heavy chain variable domain that has the amino acid sequence of SEQ ID NO: 31 and a humanized light chain variable domain derived from the light chain variable domain that has the amino acid sequence of SEQ ID NO: 32, or
(11) a humanized heavy chain variable domain derived from the heavy chain variable domain that has the amino acid sequence of SEQ ID NO: 33 and a humanized light chain variable domain derived from the light chain variable domain that has the amino acid sequence of SEQ ID NO: 34, or
(12) a humanized heavy chain variable domain derived from the heavy chain variable domain that has the amino acid sequence of SEQ ID NO: 35 and a humanized light chain variable domain derived from the light chain variable domain that has the amino acid sequence of SEQ ID NO: 36, or
(13) a humanized heavy chain variable domain derived from the heavy chain variable domain that has the amino acid sequence of SEQ ID NO: 37 and a humanized light chain variable domain derived from the light chain variable domain that has the amino acid sequence of SEQ ID NO: 38, or
(14) a humanized heavy chain variable domain derived from the heavy chain variable domain that has the amino acid sequence of SEQ ID NO: 39 and a humanized light chain variable domain derived from the light chain variable domain that has the amino acid sequence of SEQ ID NO: 40, or
(15) a humanized heavy chain variable domain derived from the heavy chain variable domain that has the amino acid sequence of SEQ ID NO: 41 and a humanized light chain variable domain derived from the light chain variable domain that has the amino acid sequence of SEQ ID NO: 42, or
(16) a humanized heavy chain variable domain derived from the heavy chain variable domain that has the amino acid sequence of SEQ ID NO: 43 and a humanized light chain variable domain derived from the light chain variable domain that has the amino acid sequence of SEQ ID NO: 44, or
(17) a humanized heavy chain variable domain derived from the heavy chain variable domain that has the amino acid sequence of SEQ ID NO: 45 and a humanized light chain variable domain derived from the light chain variable domain that has the amino acid sequence of SEQ ID NO: 46, or
(18) a humanized heavy chain variable domain derived from the heavy chain variable domain that has the amino acid sequence of SEQ ID NO: 47 and a humanized light chain variable domain derived from the light chain variable domain that has the amino acid sequence of SEQ ID NO: 48, or
(19) a humanized heavy chain variable domain derived from the heavy chain variable domain that has the amino acid sequence of SEQ ID NO: 49 and a humanized light chain variable domain derived from the light chain variable domain that has the amino acid sequence of SEQ ID NO: 50, or
(20) a humanized heavy chain variable domain derived from the heavy chain variable domain that has the amino acid sequence of SEQ ID NO: 51 and a humanized light chain variable domain derived from the light chain variable domain that has the amino acid sequence of SEQ ID NO: 52, or
(21) a humanized heavy chain variable domain derived from the heavy chain variable domain that has the amino acid sequence of SEQ ID NO: 53 and a humanized light chain variable domain derived from the light chain variable domain that has the amino acid sequence of SEQ ID NO: 54, or
(22) a humanized heavy chain variable domain derived from the heavy chain variable domain that has the amino acid sequence of SEQ ID NO: 55 and a humanized light chain variable domain derived from the light chain variable domain that has the amino acid sequence of SEQ ID NO: 56, or
(23) a humanized heavy chain variable domain derived from the heavy chain variable domain that has the amino acid sequence of SEQ ID NO: 57 and a humanized light chain variable domain derived from the light chain variable domain that has the amino acid sequence of SEQ ID NO: 58, or
(24) a humanized heavy chain variable domain derived from the heavy chain variable domain that has the amino acid sequence of SEQ ID NO: 59 and a humanized light chain variable domain derived from the light chain variable domain that has the amino acid sequence of SEQ ID NO: 60, or
(25) a humanized heavy chain variable domain derived from the heavy chain variable domain that has the amino acid sequence of SEQ ID NO: 61 and a humanized light chain variable domain derived from the light chain variable domain that has the amino acid sequence of SEQ ID NO: 62, or
(26) a humanized heavy chain variable domain derived from the heavy chain variable domain that has the amino acid sequence of SEQ ID NO: 63 and a humanized light chain variable domain derived from the light chain variable domain that has the amino acid sequence of SEQ ID NO: 64, or
(27) a humanized heavy chain variable domain derived from the heavy chain variable domain that has the amino acid sequence of SEQ ID NO: 65 and a humanized light chain variable domain derived from the light chain variable domain that has the amino acid sequence of SEQ ID NO: 66, or
(28) a humanized heavy chain variable domain derived from the heavy chain variable domain that has the amino acid sequence of SEQ ID NO: 67 and a humanized light chain variable domain derived from the light chain variable domain that has the amino acid sequence of SEQ ID NO: 68, or
(29) a humanized heavy chain variable domain derived from the heavy chain variable domain that has the amino acid sequence of SEQ ID NO: 69 and a humanized light chain variable domain derived from the light chain variable domain that has the amino acid sequence of SEQ ID NO: 70, or
(30) a humanized heavy chain variable domain derived from the heavy chain variable domain that has the amino acid sequence of SEQ ID NO: 71 and a humanized light chain variable domain derived from the light chain variable domain that has the amino acid sequence of SEQ ID NO: 72, or
(31) a humanized heavy chain variable domain derived from the heavy chain variable domain that has the amino acid sequence of SEQ ID NO: 73 and a humanized light chain variable domain derived from the light chain variable domain that has the amino acid sequence of SEQ ID NO: 74, or
(32) a humanized heavy chain variable domain derived from the heavy chain variable domain that has the amino acid sequence of SEQ ID NO: 75 and a humanized light chain variable domain derived from the light chain variable domain that has the amino acid sequence of SEQ ID NO: 76, or
(33) a humanized heavy chain variable domain derived from the heavy chain variable domain that has the amino acid sequence of SEQ ID NO: 77 and a humanized light chain variable domain derived from the light chain variable domain that has the amino acid sequence of SEQ ID NO: 78, or
(34) a humanized heavy chain variable domain derived from the heavy chain variable domain that has the amino acid sequence of SEQ ID NO: 79 and a humanized light chain variable domain derived from the light chain variable domain that has the amino acid sequence of SEQ ID NO: 80, or
(35) a humanized heavy chain variable domain derived from the heavy chain variable domain that has the amino acid sequence of SEQ ID NO: 81 and a humanized light chain variable domain derived from the light chain variable domain that has the amino acid sequence of SEQ ID NO: 82, or
(36) a humanized heavy chain variable domain derived from the heavy chain variable domain that has the amino acid sequence of SEQ ID NO: 83 and a humanized light chain variable domain derived from the light chain variable domain that has the amino acid sequence of SEQ ID NO: 84, or
(37) a humanized heavy chain variable domain derived from the heavy chain variable domain that has the amino acid sequence of SEQ ID NO: 85 and a humanized light chain variable domain derived from the light chain variable domain that has the amino acid sequence of SEQ ID NO: 86, or
(38) a humanized heavy chain variable domain derived from the heavy chain variable domain that has the amino acid sequence of SEQ ID NO: 87 and a humanized light chain variable domain derived from the light chain variable domain that has the amino acid sequence of SEQ ID NO: 88, or
(39) a humanized heavy chain variable domain derived from the heavy chain variable domain that has the amino acid sequence of SEQ ID NO: 89 and a humanized light chain variable domain derived from the light chain variable domain that has the amino acid sequence of SEQ ID NO: 90, or
(40) a humanized heavy chain variable domain derived from the heavy chain variable domain that has the amino acid sequence of SEQ ID NO: 91 and a humanized light chain variable domain derived from the light chain variable domain that has the amino acid sequence of SEQ ID NO: 92.

(41) a humanized heavy chain variable domain derived from the heavy chain variable domain that has the amino acid sequence of SEQ ID NO: 93 and a humanized light chain variable domain derived from the light chain variable domain that has the amino acid sequence of SEQ ID NO: 94, or
(42) a humanized heavy chain variable domain derived from the heavy chain variable domain that has the amino acid sequence of SEQ ID NO: 95 and a humanized light chain variable domain derived from the light chain variable domain that has the amino acid sequence of SEQ ID NO: 96, or
(43) a humanized heavy chain variable domain derived from the heavy chain variable domain that has the amino acid sequence of SEQ ID NO: 97 and a humanized light chain variable domain derived from the light chain variable domain that has the amino acid sequence of SEQ ID NO: 98.

One preferred aspect as reported herein is a humanized anti-transferrin receptor antibody or transferrin receptor binding fragment thereof comprising a humanized heavy chain variable domain derived from the heavy chain variable domain that has the amino acid sequence of SEQ ID NO: 23 and a humanized light chain variable domain derived from the light chain variable domain that has the amino acid sequence of SEQ ID NO: 24.

One aspect as reported herein is a humanized anti-human transferrin receptor antibody comprising (a) a HVR-H1 comprising the amino acid sequence of SEQ ID NO: 109; (b) a HVR-H2 comprising the amino acid sequence of SEQ ID NO: 110; and (c) a HVRH3 comprising the amino acid sequence of SEQ ID NO: 112. In one embodiment the antibody further comprises (d) a HVR-L1 comprising the amino acid sequence of SEQ ID NO: 113; (e) a HVR-L2 comprising the amino acid sequence of SEQ ID NO: 114; and (f) a HVR-L3 comprising the amino acid sequence of SEQ ID NO: 115.

One aspect as reported herein is a humanized anti-human transferrin receptor antibody comprising (a) a HVR-H1 comprising the amino acid sequence of SEQ ID NO: 109; (b) a HVR-H2 comprising the amino acid sequence of SEQ ID NO: 111; and (c) a HVRH3 comprising the amino acid sequence of SEQ ID NO: 112. In one embodiment the antibody further comprises (d) a HVR-L1 comprising the amino acid sequence of SEQ ID NO: 113; (e) a HVR-L2 comprising the amino acid sequence of SEQ ID NO: 114; and (f) a HVR-L3 comprising the amino acid sequence of SEQ ID NO: 115.

In one aspect, the invention provides an anti-transferrin receptor antibody comprising at least one, two, three, four, five, or six HVRs selected from (a) a HVR-H1 comprising the amino acid sequence of SEQ ID NO: 109; (b) a HVR-H2 comprising the amino acid sequence of SEQ ID NO: 110; (c) a HVR-H3 comprising the amino acid sequence of SEQ ID NO: 112; (d) a HVR-L1 comprising the amino acid sequence of SEQ ID NO: 113; (e) a HVR-L2 comprising the amino acid sequence of SEQ ID NO: 114; and (f) a HVR-L3 comprising the amino acid sequence of SEQ ID NO: 115.

In one aspect, the invention provides an antibody comprising at least one, at least two, or all three VH HVR sequences selected from (a) a HVR-H1 comprising the amino acid sequence of SEQ ID NO: 109; (b) a HVR-H2 comprising the amino acid sequence of SEQ ID NO: 110; and (c) a HVR-H3 comprising the amino acid sequence of SEQ ID NO: 112. In one embodiment, the antibody comprises a HVR-H3 comprising the amino acid sequence of SEQ ID NO: 112. In another embodiment, the antibody comprises a HVR-H3 comprising the amino acid sequence of SEQ ID NO: 112 and a HVR-L3 comprising the amino acid sequence of SEQ ID NO: 115. In a further embodiment, the antibody comprises a HVR-H3 comprising the amino acid sequence of SEQ ID NO: 112, a HVR-L3 comprising the amino acid sequence of SEQ ID NO: 115, and a HVRH2 comprising the amino acid sequence of SEQ ID NO: 111. In a further embodiment, the antibody comprises (a) a HVR-H1 comprising the amino acid sequence of SEQ ID NO: 109; (b) a HVR-H2 comprising the amino acid sequence of SEQ ID NO: 111; and (c) a HVR-H3 comprising the amino acid sequence of SEQ ID NO: 112.

In another aspect, the invention provides an antibody comprising at least one, at least two, or all three VL HVR sequences selected from (a) a HVR-L1 comprising the amino acid sequence of SEQ ID NO: 113; (b) a HVR-L2 comprising the amino acid sequence of SEQ ID NO: 114; and (c) a HVR-L3 comprising the amino acid sequence of SEQ ID NO: 115. In one embodiment, the antibody comprises (a) a HVR-L1 comprising the amino acid sequence of SEQ ID NO: 113; (b) a HVR-L2 comprising the amino acid sequence of SEQ ID NO: 114; and (c) a HVR-L3 comprising the amino acid sequence of SEQ ID NO: 115.

In another aspect, an antibody of the invention comprises (a) a VH domain comprising at least one, at least two, or all three VH HVR sequences selected from (i) a HVR-H1 comprising the amino acid sequence of SEQ ID NO: 109, (ii) a HVR-H2 comprising the amino acid sequence of SEQ ID NO: 110, and (iii) a HVR-H3 comprising an amino acid sequence selected from SEQ ID NO: 112; and (b) a VL domain comprising at least one, at least two, or all three VL HVR sequences selected from (i) a HVR-L1 comprising the amino acid sequence of SEQ ID NO: 113, (ii) a HVR-L2 comprising the amino acid sequence of SEQ ID NO: 114, and (c) a HVR-L3 comprising the amino acid sequence of SEQ ID NO: 115.

In another aspect, the invention provides an antibody comprising (a) a HVR-H1 comprising the amino acid sequence of SEQ ID NO: 109; (b) a HVR-H2 comprising the amino acid sequence of SEQ ID NO: 110; (c) a HVR-H3 comprising the amino acid sequence of SEQ ID NO: 112; (d) a HVR-L1 comprising the amino acid sequence of SEQ ID NO: 113; (e) a HVR-L2 comprising the amino acid sequence of SEQ ID NO: 114; and (f) a HVR-L3 comprising an amino acid sequence selected from SEQ ID NO: 115.

One preferred aspect as reported herein is a humanized anti-transferrin receptor antibody or transferrin receptor binding fragment thereof comprising a humanized heavy chain variable domain derived from the heavy chain variable domain that has the amino acid sequence of SEQ ID NO: 91 and a humanized light chain variable domain derived from the light chain variable domain that has the amino acid sequence of SEQ ID NO: 92.

One aspect as reported herein is a humanized anti-human transferrin receptor antibody comprising (a) a HVR-H1 comprising the amino acid sequence of SEQ ID NO: 116; (b) a HVR-H2 comprising the amino acid sequence of SEQ ID NO: 117; and (c) a HVRH3 comprising the amino acid sequence of SEQ ID NO: 119. In one embodiment the antibody further comprises (d) a HVR-L1 comprising the amino acid sequence of SEQ ID NO: 120; (e) a HVR-L2 comprising the amino acid sequence of SEQ ID NO: 121; and (f) a HVR-L3 comprising the amino acid sequence of SEQ ID NO: 122.

One aspect as reported herein is a humanized anti-human transferrin receptor antibody comprising (a) a HVR-H1 comprising the amino acid sequence of SEQ ID NO: 116; (b) a HVR-H2 comprising the amino acid sequence of SEQ ID NO: 118; and (c) a HVRH3 comprising the amino acid sequence of SEQ ID NO: 119. In one embodiment the antibody further comprises (d) a HVR-L1 comprising the amino acid sequence of SEQ ID NO: 120; (e) a HVR-L2 comprising the amino acid sequence of SEQ ID NO: 121; and (f) a HVR-L3 comprising the amino acid sequence of SEQ ID NO: 122.

In one aspect, the invention provides an anti-transferrin receptor antibody comprising at least one, two, three, four, five, or six HVRs selected from (a) a HVR-H1 comprising the amino acid sequence of SEQ ID NO: 116; (b) a HVR-H2 comprising the amino acid sequence of SEQ ID NO: 117; (c) a HVR-H3 comprising the amino acid sequence of SEQ ID NO: 119; (d) a HVR-L1 comprising the amino acid sequence of SEQ ID NO: 120; (e) a HVR-L2 comprising the amino acid sequence of SEQ ID NO: 121; and (f) a HVR-L3 comprising the amino acid sequence of SEQ ID NO: 122.

In one aspect, the invention provides an antibody comprising at least one, at least two, or all three VH HVR sequences selected from (a) a HVR-H1 comprising the amino acid sequence of SEQ ID NO: 116; (b) a HVR-H2 comprising the amino acid sequence of SEQ ID NO: 117; and (c) a HVR-H3 comprising the amino acid sequence of SEQ ID NO: 119. In one embodiment, the antibody comprises a HVR-H3 comprising the amino acid sequence of SEQ ID NO: 119. In another embodiment, the antibody comprises a HVR-H3 comprising the amino acid sequence of SEQ ID NO: 119 and a HVR-L3 comprising the amino acid sequence of SEQ ID NO: 122. In a further embodiment, the antibody comprises a HVR-H3 comprising the amino acid sequence of SEQ ID NO: 119, a HVR-L3 comprising the amino acid sequence of SEQ ID NO: 122, and a HVRH2 comprising the amino acid sequence of SEQ ID NO: 118. In a further embodiment, the antibody comprises (a) a HVR-H1 comprising the amino acid sequence of SEQ ID NO: 116; (b) a HVR-H2 comprising the amino acid sequence of SEQ ID NO: 118; and (c) a HVR-H3 comprising the amino acid sequence of SEQ ID NO: 119.

In another aspect, the invention provides an antibody comprising at least one, at least two, or all three VL HVR sequences selected from (a) a HVR-L1 comprising the amino acid sequence of SEQ ID NO: 120; (b) a HVR-L2 comprising the amino acid sequence of SEQ ID NO: 121; and (c) a HVR-L3 comprising the amino acid sequence of SEQ ID NO: 122. In one embodiment, the antibody comprises (a) a HVR-L1 comprising the amino acid sequence of SEQ ID NO: 120; (b) a HVR-L2 comprising the amino acid sequence of SEQ ID NO: 121; and (c) a HVR-L3 comprising the amino acid sequence of SEQ ID NO: 122.

In another aspect, an antibody of the invention comprises (a) a VH domain comprising at least one, at least two, or all three VH HVR sequences selected from (i) a HVR-H1 comprising the amino acid sequence of SEQ ID NO: 116, (ii) a HVR-H2 comprising the amino acid sequence of SEQ ID NO: 117, and (iii) a HVR-H3 comprising an amino acid sequence selected from SEQ ID NO: 119; and (b) a VL domain comprising at least one, at least two, or all three VL HVR sequences selected from (i) a HVR-L1 comprising the amino acid sequence of SEQ ID NO: 120, (ii) a HVR-L2 comprising the amino acid sequence of SEQ ID NO: 121, and (c) a HVR-L3 comprising the amino acid sequence of SEQ ID NO: 122.

In another aspect, the invention provides an antibody comprising (a) a HVR-H1 comprising the amino acid sequence of SEQ ID NO: 116; (b) a HVR-H2 comprising the amino acid sequence of SEQ ID NO: 117; (c) a HVR-H3 comprising the amino acid sequence of SEQ ID NO: 119; (d) a HVR-L1 comprising the amino acid sequence of SEQ ID NO: 120; (e) a HVR-L2 comprising the amino acid sequence of SEQ ID NO: 121; and (f) a HVR-L3 comprising an amino acid sequence selected from SEQ ID NO: 122.

One aspect as reported herein is a transferrin shuttle module comprising a full length IgG antibody as brain effector entity, a linker and one scFab as the monovalent binding entity, which binds the transferrin receptor, wherein the scFab is conjugated to the C-terminal end of the Fc-region of one of the heavy chains of the IgG antibody via the linker.

One aspect as reported herein is a transferrin shuttle module comprising a full length IgG antibody as brain effector entity, a linker and one scFv as the monovalent binding entity, which binds the transferrin barrier receptor, wherein the scFv is conjugated to the C-terminal end of the Fc-region of one of the heavy chains of the IgG antibody via the linker.

In one preferred embodiment the monovalent binding entity comprises the HVRs of SEQ ID NOs:109, 110, 112, 113, 114, and 115, or of SEQ ID NOs:116, 117, 119, 120, 121, and 122.

In a further aspect, an anti-transferrin receptor antibody according to any of the above embodiments may incorporate any of the features, singly or in combination, as described in Sections 1-6 below:

### 1. Antibody Affinity

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen, Y. et al., J. Mol. Biol. 293 (1999) 865-881). To establish conditions for the assay, MICROTITER® multi-well plates (Thermo Scientific) are coated overnight with 5 μg/mL of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta, L. G. et al., Cancer Res. 57 (1997) 4593-4599). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μL/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (˜0.2 μM) before injection at a flow rate of 5 μL/minute to achieve approximately 10 response units (RU) of coupled protein. Following the injection of antigen, 1 M ethanolamine is injected to block non-reacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ (see, e.g., Chen, Y. et al., J. Mol. Biol. 293 (1999) 865-881). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm bandpass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLMAMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson, P. J. et al., Nat. Med. 9 (2003) 129-134. For a review of scFv fragments, see, e.g., Plueckthun, A., In; The Pharmacology of Monoclonal Antibodies, Vol. 113, Rosenburg and Moore (eds.), Springer-Verlag, New York (1994), pp. 269-315; see also WO 93/16185; U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 0 404 097; WO 1993/01161; Hudson, P. J. et al., Nat. Med. 9 (2003) 129-134; and Holliger, P. et al., Proc. Natl. Acad. Sci. USA 90 (1993) 6444-6448. Triabodies and tetrabodies are also described in Hudson, P. J. et al., Nat. Med. 9 (20039 129-134).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, M A; see, e.g., U.S. Pat. No. 6,248,516).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison, S. L. et al., Proc. Natl. Acad. Sci. USA 81 (1984) 6851-6855). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro, J. C. and Fransson, J., Front. Biosci. 13 (2008) 1619-1633, and are further described, e.g., in Riechmann, I. et al., Nature 332 (1988) 323-329; Queen, C. et al., Proc. Natl. Acad. Sci. USA 86 (1989) 10029-10033; U.S. Pat. Nos. 5,821,337, 7,527,791, 6,982,321, and 7,087,409; Kashmiri, S. V. et al., Methods 36 (2005) 25-34 (describing specificity determining region (SDR) grafting); Padlan, E. A., Mol. Immunol. 28 (1991) 489-498 (describing “resurfacing”); Dall'Acqua, W. F. et al., Methods 36 (2005) 43-60 (describing “FR shuffling”); and Osbourn, J. et al., Methods 36 (2005) 61-68 and Klimka, A. et al., Br. J. Cancer 83 (2000) 252-260 (describing the “guided selection” approach to FR shuffling).

Human framework regions that may be used for humanization include but are not limited to: framework regions selected using the “best-fit” method (see, e.g., Sims, M. J. et al., J. Immunol. 151 (1993) 2296-2308; framework regions derived from the consensus sequence of human antibodies of a particular subgroup of light or heavy chain variable regions (see, e.g., Carter, P. et al., Proc. Natl. Acad. Sci. USA 89 (1992) 4285-4289; and Presta, L. G. et al., J. Immunol. 151 (1993) 2623-2632); human mature (somatically mutated) framework regions or human germline framework regions (see, e.g., Almagro, J. C. and Fransson, J., Front. Biosci. 13 (2008) 1619-1633); and framework regions derived from screening FR libraries (see, e.g., Baca, M. et al., J. Biol. Chem. 272 (1997) 10678-10684 and Rosok, M. J. et al., J. Biol. Chem. 271 (19969 22611-22618).

### 4. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom, H. R. et al., Methods in Molecular Biology 178 (2001) 1-37 and further described, e.g., in the McCafferty, J. et al., Nature 348 (1990) 552-554; Clackson, T. et al., Nature 352 (1991) 624-628; Marks, J. D. et al., J. Mol. Biol. 222 (1992) 581-597; Marks, J. D. and Bradbury, A., Methods in Molecular Biology 248 (2003) 161-175; Sidhu, S. S. et al., J. Mol. Biol. 338 (2004) 299-310; Lee, C. V. et al., J. Mol. Biol. 340 (2004) 1073-1093; Fellouse, F. A., Proc. Natl. Acad. Sci. USA 101 (2004) 12467-12472; and Lee, C. V. et al., J. Immunol. Methods 284 (2004) 119-132.

In certain phage display methods, repertoires of VH and VL genes are separately cloned by polymerase chain reaction (PCR) and recombined randomly in phage libraries, which can then be screened for antigen-binding phage as described in Winter, G. et al., Ann. Rev. Immunol. 12 (1994) 433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also selfantigens without any immunization as described by Griffiths, A. D. et al., EMBO J. 12 (1993) 725-734. Finally, naive libraries can also be made synthetically by cloning nonrearranged V-gene segments from stem cells, and using PCR primers containing random sequence to encode the highly variable CDR3 regions and to accomplish rearrangement in vitro, as described by Hoogenboom, H. R. and Winter, G., J. Mol. Biol. 227 (1992) 381-388. Patent publications describing human antibody phage libraries include, for example: U.S. Pat. No. 5,750,373, and US 2005/0079574, US 2005/0119455, US 2005/0266000, US 2007/0117126, US 2007/0160598, US 2007/0237764, US 2007/0292936, and US 2009/0002360.

### 5. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for the transferrin receptor and the other is for any other antigen. Bispecific antibodies may also be used to localize cytotoxic agents to cells which express the transferrin receptor. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knobin-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making bispecific antibody fragments (see, e.g., Holliger, P. et al., Proc. Natl. Acad. Sci. USA 90 (1993) 6444-6448); and using single-chain Fv (scFv) dimers (see, e.g. Gruber, M et al., J. Immunol. 152 (1994) 5368-5374); and preparing trispecific antibodies as described, e.g., in Tutt, A. et al., J. Immunol. 147 (1991) 60-69).

Engineered antibodies with three or more functional antigen binding sites, including “Octopus antibodies”, are also included herein (see, e.g. US 2006/0025576).

The antibody or fragment herein also includes a “Dual Acting Fab” or “DAF” comprising an antigen binding site that binds to the transferrin receptor as well as another, different antigen (see, US 2008/0069820, for example).

The antibody or fragment herein also includes multispecific antibodies described in WO 2009/080251, WO 2009/080252, WO 2009/080253, WO 2009/080254, WO 2010/112193, WO 2010/115589, WO 2010/136172, WO 2010/145792, and WO 2010/145793.

In one embodiment of all aspects as reported herein the anti-transferrin receptor antibody is a bispecific antibody.

One aspect as reported herein is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other,
wherein the first antigen or the second antigen is human transferrin receptor.

The antibody under a) does not contain a modification as reported under b) and the heavy chain and the light chain under a) are isolated chains.

In the antibody under b) within the light chain
the variable light chain domain VL is replaced by the variable heavy chain domain VH of said antibody,
and
within the heavy chain, the variable heavy chain domain VH is replaced by the variable light chain domain VL of said antibody.

In one embodiment
i) in the constant domain CL of the first light chain under a) the amino acid at position 124 (numbering according to Kabat) is substituted by a positively charged amino acid, and wherein in the constant domain CH1 of the first heavy chain under a) the amino acid at position 147 or the amino acid at position 213 (numbering according to Kabat EU index) is substituted by a negatively charged amino acid,
or
ii) in the constant domain CL of the second light chain under b) the amino acid at position 124 (numbering according to Kabat) is substituted by a positively charged amino acid, and wherein in the constant domain CH1 of the second heavy chain under b) the amino acid at position 147 or the amino acid at position 213 (numbering according to Kabat EU index) is substituted by a negatively charged amino acid.

In one preferred embodiment
i) in the constant domain CL of the first light chain under a) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the first heavy chain under a) the amino acid at position 147 or the amino acid at position 213 is substituted independently by glutamic acid (E) or aspartic acid (D) (numbering according to Kabat EU index),
or
ii) in the constant domain CL of the second light chain under b) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the second heavy chain under b) the amino acid at position 147 or the amino acid at position 213 is substituted independently by glutamic acid (E) or aspartic acid (D) (numbering according to Kabat EU index).

In one embodiment in the constant domain CL of the second heavy chain the amino acids at position 124 and 123 are substituted by K (numbering according to Kabat EU index).

In one embodiment in the constant domain CH1 of the second light chain the amino acids at position 147 and 213 are substituted by E (numbering according to EU index of Kabat).

In one preferred embodiment in the constant domain CL of the first light chain the amino acids at position 124 and 123 are substituted by K, and in the constant domain CH1 of the first heavy chain the amino acids at position 147 and 213 are substituted by E (numbering according to Kabat EU index).

In one embodiment in the constant domain CL of the second heavy chain the amino acids at position 124 and 123 are substituted by K, and wherein in the constant domain CH1 of the second light chain the amino acids at position 147 and 213 are substituted by E, and in the variable domain VL of the first light chain the amino acid at position 38 is substituted by K, in the variable domain VH of the first heavy chain the amino acid at position 39 is substituted by E, in the variable domain VL of the second heavy chain the amino acid at position 38 is substituted by K, and in the variable domain VH of the second light chain the amino acid at position 39 is substituted by E (numbering according to Kabat EU index).

One aspect as reported herein is a bivalent, bispecific antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other, and wherein the constant domains CL and CH1 of the second light chain and the second heavy chain are replaced by each other,
wherein the first antigen or the second antigen is human transferrin receptor.

The antibody under a) does not contain a modification as reported under b) and the heavy chain and the light chain und a) are isolated chains.

In the antibody under b) within the light chain
the variable light chain domain VL is replaced by the variable heavy chain domain VH of said antibody, and the constant light chain domain CL is replaced by the constant heavy chain domain CH1 of said antibody;
and
within the heavy chain, the variable heavy chain domain VH is replaced by the variable light chain domain VL of said antibody, and the constant heavy chain domain CH1 is replaced by the constant light chain domain CL of said antibody.

One aspect as reported herein is a bivalent, bispecific antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other,
wherein the first antigen or the second antigen is human transferrin receptor.

The antibody under a) does not contain a modification as reported under b) and the heavy chain and the light chain under a) are isolated chains.

In the antibody under b)
within the light chain the constant light chain domain CL is replaced by the constant heavy chain domain CH1 of said antibody;
and within the heavy chain, the constant heavy chain domain CH1 is replaced by the constant light chain domain CL of said antibody.

One aspect as reported herein is a multispecific antibody comprising
a) a full length antibody specifically binding to a first antigen and consisting of two antibody heavy chains and two antibody light chains, and
b) one, two, three or four single chain Fab fragments specifically binding to one to four further antigens (i.e. a second and/or third and/or fourth and/or fifth antigen, preferably specifically binding to one further antigen, i.e. a second antigen),
wherein said single chain Fab fragments under b) are fused to said full length antibody under a) via a peptidic linker at the C- or N-terminus of the heavy or light chain of said full length antibody,
wherein the first antigen or one of the further antigens is human transferrin receptor.

In one embodiment one or two identical single chain Fab fragments binding to a second antigen are fused to said full length antibody via a peptidic linker at the C-terminus of the heavy or light chains of said full length antibody.

In one embodiment one or two identical single chain Fab fragments binding to a second antigen are fused to said full length antibody via a peptidic linker at the C-terminus of the heavy chains of said full length antibody.

In one embodiment one or two identical single chain Fab fragments binding to a second antigen are fused to said full length antibody via a peptidic linker at the C-terminus of the light chains of said full length antibody.

In one embodiment two identical single chain Fab fragments binding to a second antigen are fused to said full length antibody via a peptidic linker at the C-terminus of each heavy or light chain of said full length antibody.

In one embodiment two identical single chain Fab fragments binding to a second antigen are fused to said full length antibody via a peptidic linker at the C-terminus of each heavy chain of said full length antibody.

In one embodiment two identical single chain Fab fragments binding to a second antigen are fused to said full length antibody via a peptidic linker at the C-terminus of each light chain of said full length antibody.

One aspect as reported herein is a trivalent, bispecific antibody comprising
a) a full length antibody specifically binding to a first antigen and consisting of two antibody heavy chains and two antibody light chains,
b) a first polypeptide consisting of
   ba) an antibody heavy chain variable domain (VH),
   or
   bb) an antibody heavy chain variable domain (VH) and an antibody constant domain 1 (CH1),
   wherein said first polypeptide is fused with the N-terminus of its VH domain via a peptidic linker to the C-terminus of one of the two heavy chains of said full length antibody,
c) a second polypeptide consisting of
   ca) an antibody light chain variable domain (VL),
   or
   cb) an antibody light chain variable domain (VL) and an antibody light chain constant domain (CL),
   wherein said second polypeptide is fused with the N-terminus of the VL domain via a peptidic linker to the C-terminus of the other of the two heavy chains of said full length antibody,
and
wherein the antibody heavy chain variable domain (VH) of the first polypeptide and the antibody light chain variable domain (VL) of the second polypeptide together form an antigen-binding site specifically binding to a second antigen,
and
wherein the first antigen or the second antigen is human transferrin receptor.

In one embodiment the antibody heavy chain variable domain (VH) of the polypeptide under b) and the antibody light chain variable domain (VL) of the polypeptide under c) are linked and stabilized via an interchain disulfide bridge by introduction of a disulfide bond between the following positions:
i) heavy chain variable domain position 44 to light chain variable domain position 100, or
ii) heavy chain variable domain position 105 to light chain variable domain position 43, or
iii) heavy chain variable domain position 101 to light chain variable domain position 100 (numbering always according to Kabat EU index).

Techniques to introduce unnatural disulfide bridges for stabilization are described e.g. in WO 94/029350, Rajagopal, V., et al., Prot. Eng. (1997) 1453-59; Kobayashi, H., et al., Nuclear Medicine & Biology, Vol. 25, (1998) 387-393; or Schmidt, M., et al., Oncogene (1999) 18 1711-1721. In one embodiment the optional disulfide bond between the variable domains of the polypeptides under b) and c) is between heavy chain variable domain position 44 and light chain variable domain position 100. In one embodiment the optional disulfide bond between the variable domains of the polypeptides under b) and c) is between heavy chain variable domain position 105 and light chain variable domain position 43 (numbering always according to EU index of Kabat). In one embodiment a trivalent, bispecific antibody without said optional disulfide stabilization between the variable domains VH and VL of the single chain Fab fragments is preferred.

One aspect as reported herein is a trispecific or tetraspecific antibody, comprising
a) a first light chain and a first heavy chain of a full length antibody which specifically binds to a first antigen, and
b) a second (modified) light chain and a second (modified) heavy chain of a full length antibody which specifically binds to a second antigen, wherein the variable domains VL and VH are replaced by each other, and/or wherein the constant domains CL and CH1 are replaced by each other, and
c) wherein one to four antigen binding peptides which specifically bind to one or two further antigens (i.e. to a third and/or fourth antigen) are fused via a peptidic linker to the C- or N-terminus of the light chains or heavy chains of a) and/or b),
wherein the first antigen or the second antigen or one of the further antigens is human transferrin receptor.

The antibody under a) does not contain a modification as reported under b) and the heavy chain and the light chain und a) are isolated chains.

In one embodiment the trispecific or tetraspecific antibody comprises under c) one or two antigen binding peptides which specifically bind to one or two further antigens.

In one embodiment the antigen binding peptides are selected from the group of a scFv fragment and a scFab fragment.

In one embodiment the antigen binding peptides are scFv fragments.

In one embodiment the antigen binding peptides are scFab fragments.

In one embodiment the antigen binding peptides are fused to the C-terminus of the heavy chains of a) and/or b).

In one embodiment the trispecific or tetraspecific antibody comprises under c) one or two antigen binding peptides which specifically bind to one further antigen.

In one embodiment the trispecific or tetraspecific antibody comprises under c) two identical antigen binding peptides which specifically bind to a third antigen. In one preferred embodiment such two identical antigen binding peptides are fused both via the same peptidic linker to the C-terminus of the heavy chains of a) and b). In one preferred embodiment the two identical antigen binding peptides are either a scFv fragment or a scFab fragment.

In one embodiment the trispecific or tetraspecific antibody comprises under c) two antigen binding peptides which specifically bind to a third and a fourth antigen. In one embodiment said two antigen binding peptides are fused both via the same peptide connector to the C-terminus of the heavy chains of a) and b). In one preferred embodiment said two antigen binding peptides are either a scFv fragment or a scFab fragment.

One aspect as reported herein is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a),
and
wherein in the Fab fragments the following modifications were performed
   i) in both Fab fragments of a), or in both Fab fragments of b), the variable domains VL and VH are replaced by each other, and/or the constant domains CL and CH1 are replaced by each other,
   or
   ii) in both Fab fragments of a) the variable domains VL and VH are replaced by each other, and the constant domains CL and CH1 are replaced by each other,
      and
      in both Fab fragments of b) the variable domains VL and VH are replaced by each other, or the constant domains CL and CH1 are replaced by each other,
   or
   iii) in both Fab fragments of a) the variable domains VL and VH are replaced by each other, or the constant domains CL and CH1 are replaced by each other,
      and
      in both Fab fragments of b) the variable domains VL and VH are replaced by each other, and the constant domains CL and CH1 are replaced by each other,
   or
   iv) in both Fab fragments of a) the variable domains VL and VH are replaced by each other, and in both Fab fragments of b) the constant domains CL and CH1 are replaced by each other,
   or
   v) in both Fab fragments of a) the constant domains CL and CH1 are replaced by each other, and in both Fab fragments of b) the variable domains VL and VH are replaced by each other,
wherein the first antigen or the second antigen is human transferrin receptor.

In one embodiment said additional Fab fragments are fused both via a peptidic linker either to the C-termini of the heavy chains of a), or to the N-termini of the heavy chains of a).

In one embodiment said additional Fab fragments are fused both via a peptidic linker either to the C-termini of the heavy chains of a).

In one embodiment said additional Fab fragments are fused both via a peptide connector to the N-termini of the heavy chains of a).

In one embodiment in the Fab fragments the following modifications are performed:
i) in both Fab fragments of a), or in both Fab fragments of b), the variable domains VL and VH are replaced by each other, and/or the constant domains CL and CH1 are replaced by each other.

In one embodiment in the Fab fragments the following modifications are performed:
i) in both Fab fragments of a) the variable domains VL and VH are replaced by each other, and/or the constant domains CL and CH1 are replaced by each other.

In one embodiment in the Fab fragments the following modifications are performed:
i) in both Fab fragments of a) the constant domains CL and CH1 are replaced by each other.

In one embodiment in the Fab fragments the following modifications are performed:
i) in both Fab fragments of b) the variable domains VL and VH are replaced by each other, and/or the constant domains CL and CH1 are replaced by each other.

In one embodiment in the Fab fragments the following modifications are performed:
i) in both Fab fragments of b) the constant domains CL and CH1 are replaced by each other.

One aspect as reported herein is a bispecific, tetravalent antibody comprising:
a) a (modified) heavy chain of a first antibody, which specifically binds to a first antigen and comprises a first VH-CH1 domain pair, wherein to the C-terminus of said heavy chain the N-terminus of a second VH-CH1 domain pair of said first antibody is fused via a peptidic linker,
b) two light chains of said first antibody of a),
c) a (modified) heavy chain of a second antibody, which specifically binds to a second antigen and comprises a first VH-CL domain pair, wherein to the C-terminus of said heavy chain the N-terminus of a second VH-CL domain pair of said second antibody is fused via a peptidic linker, and
d) two (modified) light chains of said second antibody of c), each comprising a CL-CH1 domain pair,
wherein the first antigen or the second antigen is human transferrin receptor.

One aspect as reported herein is a bispecific antibody comprising
a) the heavy chain and the light chain of a first full length antibody that specifically binds to a first antigen, and
b) the heavy chain and the light chain of a second full length antibody that specifically binds to a second antigen, wherein the N-terminus of the heavy chain is connected to the C-terminus of the light chain via a peptidic linker,
wherein the first antigen or the second antigen is human transferrin receptor.

The antibody under a) does not contain a modification as reported under b) and the heavy chain and the light chain are isolated chains.

One aspect as reported herein is a bispecific antibody comprising
a) a full length antibody specifically binding to a first antigen and consisting of two antibody heavy chains and two antibody light chains, and
b) an Fv fragment specifically binding to a second antigen comprising a VH² domain and a VL² domain, wherein both domains are connected to each other via a disulfide bridge,
wherein only either the VH² domain or the VL² domain is fused via a peptidic linker to the heavy or light chain of the full length antibody specifically binding to a first antigen,
wherein the first antigen or the second antigen is human transferrin receptor.

In the bispecific the heavy chains and the light chains under a) are isolated chains.

In one embodiment the other of the VH² domain or the VL² domain is not fused via a peptide linker to the heavy or light chain of the full length antibody specifically binding to a first antigen.

In all aspects as reported herein the first light chain comprises a VL domain and a CL domain and the first heavy chain comprises a VH domain, a CH1 domain, a hinge region, a CH2 domain and a CH3 domain.

In one embodiment of all aspects the antibody as reported herein is a multi-specific antibody, which requires heterodimerization of at least two heavy chain polypeptides, and wherein the antibody specifically binds to human transferrin receptor and a second non-human transferrin receptor antigen.

Several approaches for CH3-modifications in order to support heterodimerization have been described, for example in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, which are herein included by reference. Typically, in the approaches known in the art, the CH3 domain of the first heavy chain and the CH3 domain of the second heavy chain are both engineered in a complementary manner so that the heavy chain comprising one engineered CH3 domain can no longer homodimerize with another heavy chain of the same structure (e.g. a CH3-engineered first heavy chain can no longer homodimerize with another CH3-engineered first heavy chain; and a CH3-engineered second heavy chain can no longer homodimerize with another CH3-engineered second heavy chain). Thereby the heavy chain comprising one engineered CH3 domain is forced to heterodimerize with another heavy chain comprising the CH3 domain, which is engineered in a complementary manner. For this embodiment of the invention, the CH3 domain of the first heavy chain and the CH3 domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g. for steric reasons).

The different approaches for supporting heavy chain heterodimerization known in the art, that were cited and included above, are contemplated as different alternatives used in a multispecific antibody according to the invention, which comprises a “non-crossed Fab region” derived from a first antibody, which specifically binds to a first antigen, and a “crossed Fab region” derived from a second antibody, which specifically binds to a second antigen, in combination with the particular amino acid substitutions described above for the invention.

The CH3 domains of the multispecific antibody as reported herein can be altered by the “knob-into-holes” technology which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

In one preferred embodiment the multispecific antibody as reported herein comprises a T366W mutation in the CH3 domain of the “knobs chain” and T366S, L368A, Y407V mutations in the CH3 domain of the “hole-chain” (numbering according to Kabat EU index). An additional interchain disulfide bridge between the CH3 domains can also be used (Merchant, A. M., et al., Nature Biotech. 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the multispecific antibody as reported herein comprises the Y349C and T366W mutations in one of the two CH3 domains and the E356C, T366S, L368A and Y407V mutations in the other of the two CH3 domains or the multispecific antibody as reported herein comprises the Y349C and T366W mutations in one of the two CH3 domains and the S354C, T366S, L368A and Y407V mutations in the other of the two CH3 domains (the additional Y349C mutation in one CH3 domain and the additional E356C or S354C mutation in the other CH3 domain forming a interchain disulfide bridge) (numbering according to Kabat EU index).

But also other knobs-in-holes technologies as described by EP 1 870 459A1, can be used alternatively or additionally. In one embodiment the multispecific antibody as reported herein comprises the R409D and K370E mutations in the CH3 domain of the “knobs chain” and the D399K and E357K mutations in the CH3 domain of the “hole-chain” (numbering according to Kabat EU index).

In one embodiment the multispecific antibody as reported herein comprises a T366W mutation in the CH3 domain of the “knobs chain” and the T366S, L368A and Y407V mutations in the CH3 domain of the “hole chain” and additionally the R409D and K370E mutations in the CH3 domain of the “knobs chain” and the D399K and E357K mutations in the CH3 domain of the “hole chain” (numbering according to the Kabat EU index).

In one embodiment the multispecific antibody as reported herein comprises the Y349C and T366W mutations in one of the two CH3 domains and the S354C, T366S, L368A and Y407V mutations in the other of the two CH3 domains, or the multispecific antibody as reported herein comprises the Y349C and T366W mutations in one of the two CH3 domains and the S354C, T366S, L368A and Y407V mutations in the other of the two CH3 domains and additionally the R409D and K370E mutations in the CH3 domain of the “knobs chain” and the D399K and E357K mutations in the CH3 domain of the “hole chain” (numbering according to the Kabat EU index).

Apart from the “knob-into-hole technology” other techniques for modifying the CH3 domains of the heavy chains of a multispecific antibody to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knob-into-hole technology” in combination with a multispecific antibody as reported herein.

In one embodiment of a multispecific antibody as reported herein the approach described in EP 1870459 is used to support heterodimerization of the first heavy chain and the second heavy chain of the multispecific antibody. This approach is based on the introduction of charged amino acids with opposite charges at specific amino acid positions in the CH3/CH3-domain-interface between both, the first and the second heavy chain.

Accordingly, this embodiment relates to a multispecific antibody as reported herein, wherein in the tertiary structure of the antibody the CH3 domain of the first heavy chain and the CH3 domain of the second heavy chain form an interface that is located between the respective antibody CH3 domains, wherein the respective amino acid sequences of the CH3 domain of the first heavy chain and the CH3 domain of the second heavy chain each comprise a set of amino acids that is located within said interface in the tertiary structure of the antibody, wherein from the set of amino acids that is located in the interface in the CH3 domain of one heavy chain a first amino acid is substituted by a positively charged amino acid and from the set of amino acids that is located in the interface in the CH3 domain of the other heavy chain a second amino acid is substituted by a negatively charged amino acid. The multispecific antibody according to this embodiment is herein also referred to as “CH3(+/−)-engineered multispecific antibody” (wherein the abbreviation “+/−” stands for the oppositely charged amino acids that were introduced in the respective CH3 domains).

In one embodiment of said CH3(+/−)-engineered multispecific antibody as reported herein the positively charged amino acid is selected from K, R and H, and the negatively charged amino acid is selected from E or D.

In one embodiment of said CH3(+/−)-engineered multispecific antibody as reported herein the positively charged amino acid is selected from K and R, and the negatively charged amino acid is selected from E or D.

In one embodiment of said CH3(+/−)-engineered multispecific antibody as reported herein the positively charged amino acid is K, and the negatively charged amino acid is E.

In one embodiment of said CH3(+/−)-engineered multispecific antibody as reported herein in the CH3 domain of one heavy chain the amino acid R at position 409 is substituted by D and the amino acid K at position is substituted by E, and in the CH3 domain of the other heavy chain the amino acid D at position 399 is substituted by K and the amino acid E at position 357 is substituted by K (numbering according to Kabat EU index).

In one embodiment of a multispecific antibody as reported herein the approach described in WO2013/157953 is used to support heterodimerization of the first heavy chain and the second heavy chain of the multispecific antibody. In one embodiment of said multispecific antibody as reported herein, in the CH3 domain of one heavy chain the amino acid T at position 366 is substituted by K, and in the CH3 domain of the other heavy chain the amino acid L at position 351 is substituted by D (numbering according to Kabat EU index). In another embodiment of said multispecific antibody as reported herein, in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index).

In another embodiment of said multispecific antibody as reported herein, in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index). Additionally at least one of the following substitutions is comprised in the CH3 domain of the other heavy chain: the amino acid Y at position 349 is substituted by E, the amino acid Y at position 349 is substituted by D and the amino acid L at position 368 is substituted by E (numbering according to Kabat EU index). In one embodiment the amino acid L at position 368 is substituted by E (numbering according to Kabat EU index).

In one embodiment of a multispecific antibody as reported herein the approach described in WO2012/058768 is used to support heterodimerization of the first heavy chain and the second heavy chain of the multispecific antibody. In one embodiment of said multispecific antibody as reported herein, in the CH3 domain of one heavy chain the amino acid L at position 351 is substituted by Y and the amino acid Y at position 407 is substituted by A, and in the CH3 domain of the other heavy chain the amino acid T at position 366 is substituted by A and the amino acid K at position 409 is substituted by F (numbering according to Kabat EU index). In another embodiment, in addition to the aforementioned substitutions, in the CH3 domain of the other heavy chain at least one of the amino acids at positions 411 (originally T), 399 (originally D), 400 (originally S), 405 (originally F), 390 (originally N) and 392 (originally K) is substituted (numbering according to Kabat EU index). Preferred substitutions are:
substituting the amino acid T at position 411 by an amino acid selected from N, R, Q, K, D, E and W (numbering according to Kabat EU index),
substituting the amino acid D at position 399 by an amino acid selected from R, W, Y, and K (numbering according to Kabat EU index),
substituting the amino acid S at position 400 by an amino acid selected from E, D, R and K (numbering according to Kabat EU index),
substituting the amino acid F at position 405 by an amino acid selected from I, M, T, S, V and W (numbering according to Kabat EU index;
substituting the amino acid N at position 390 by an amino acid selected from R, K and D (numbering according to Kabat EU index; and
substituting the amino acid K at position 392 by an amino acid selected from V, M, R, L, F and E (numbering according to Kabat EU index).

In another embodiment of said multispecific antibody as reported herein (engineered according to WO2012/058768), in the CH3 domain of one heavy chain the amino acid L at position 351 is substituted by Y and the amino acid Y at position 407 is substituted by A, and in the CH3 domain of the other heavy chain the amino acid T at position 366 is substituted by V and the amino acid K at position 409 is substituted by F (numbering according to Kabat EU index). In another embodiment of said multispecific antibody as reported herein, in the CH3 domain of one heavy chain the amino acid Y at position 407 is substituted by A, and in the CH3 domain of the other heavy chain the amino acid T at position 366 is substituted by A and the amino acid K at position 409 is substituted by F (numbering according to Kabat EU index). In said last aforementioned embodiment, in the CH3 domain of said other heavy chain the amino acid K at position 392 is substituted by E, the amino acid T at position 411 is substituted by E, the amino acid D at position 399 is substituted by R and the amino acid S at position 400 is substituted by R (numbering according to Kabat EU index).

In one embodiment of a multispecific antibody as reported herein the approach described in WO 2011/143545 is used to support heterodimerization of the first heavy chain and the second heavy chain of the multispecific antibody. In one embodiment of said multispecific antibody as reported herein, amino acid modifications in the CH3 domains of both heavy chains are introduced at positions 368 and/or 409 (numbering according to Kabat EU index).

In one embodiment of a multispecific antibody as reported herein the approach described in WO 2011/090762 is used to support heterodimerization of the first heavy chain and the second heavy chain of the multispecific antibody. WO 2011/090762 relates to amino acid modifications according to the “knob-into-hole” technology. In one embodiment of said CH3(KiH)-engineered multispecific antibody as reported herein, in the CH3 domain of one heavy chain the amino acid T at position 366 is substituted by W, and in the CH3 domain of the other heavy chain the amino acid Y at position 407 is substituted by A (numbering according to Kabat EU index). In another embodiment of said CH3(KiH)-engineered multispecific antibody as reported herein, in the CH3 domain of one heavy chain the amino acid T at position 366 is substituted by Y, and in the CH3 domain of the other heavy chain the amino acid Y at position 407 is substituted by T (numbering according to Kabat EU index).

In one embodiment of a multispecific antibody as reported herein, which is of IgG₂ isotype, the approach described in WO 2011/090762 is used to support heterodimerization of the first heavy chain and the second heavy chain of the multispecific antibody.

In one embodiment of a multispecific antibody as reported herein, the approach described in WO 2009/089004 is used to support heterodimerization of the first heavy chain and the second heavy chain of the multispecific antibody. In one embodiment of said multispecific antibody as reported herein, in the CH3 domain of one heavy chain the amino acid K or N at position 392 is substituted by a negatively charged amino acid (in one preferred embodiment by E or D, in one preferred embodiment by D), and in the CH3 domain of the other heavy chain the amino acid D at position 399 the amino acid E or D at position 356 or the amino acid E at position 357 is substituted by a positively charged amino acid (in one preferred embodiment K or R, in one preferred embodiment by K, in one preferred embodiment the amino acids at positions 399 or 356 are substituted by K) (numbering according to Kabat EU index). In one further embodiment, in addition to the aforementioned substitutions, in the CH3 domain of the one heavy chain the amino acid K or R at position 409 is substituted by a negatively charged amino acid (in one preferred embodiment by E or D, in one preferred embodiment by D) (numbering according to Kabat EU index). In one even further embodiment, in addition to or alternatively to the aforementioned substitutions, in the CH3 domain of the one heavy chain the amino acid K at position 439 and/or the amino acid K at position 370 is substituted independently from each other by a negatively charged amino acid (in one preferred embodiment by E or D, in one preferred embodiment by D) (numbering according to Kabat EU index).

In one embodiment of a multispecific antibody as reported herein, the approach described in WO 2007/147901 is used to support heterodimerization of the first heavy chain and the second heavy chain of the multispecific antibody. In one embodiment of said multispecific antibody as reported herein, in the CH3 domain of one heavy chain the amino acid K at position 253 is substituted by E, the amino acid D at position 282 is substituted by K and the amino acid K at position 322 is substituted by D, and in the CH3 domain of the other heavy chain the amino acid D at position 239 is substituted by K, the amino acid E at position 240 is substituted by K and the amino acid K at position 292 is substituted by D (numbering according to Kabat EU index).

In one embodiment of a multispecific antibody as reported herein, the approach described in WO 2007/110205 is used to support heterodimerization of the first heavy chain and the second heavy chain of the multispecific antibody

In one embodiment of all aspects and embodiments as reported herein the multispecific antibody is a bispecific antibody or a trispecific antibody. In one preferred embodiment of the invention the multispecific antibody is a bispecific antibody.

In one embodiment of all aspects as reported herein, the antibody is a bivalent or trivalent antibody. In one embodiment the antibody is a bivalent antibody.

In one embodiment of all aspects as reported herein, the multispecific antibody has a constant domain structure of an IgG type antibody. In one further embodiment of all aspects as reported herein, the multispecific antibody is characterized in that said multispecific antibody is of human subclass IgG1, or of human subclass IgG1 with the mutations L234A and L235A. In one further embodiment of all aspects as reported herein, the multispecific antibody is characterized in that said multispecific antibody is of human subclass IgG2. In one further embodiment of all aspects as reported herein, the multispecific antibody is characterized in that said multispecific antibody is of human subclass IgG3. In one further embodiment of all aspects as reported herein, the multispecific antibody is characterized in that said multispecific antibody is of human subclass IgG4 or, of human subclass IgG4 with the additional mutation S228P. In one further embodiment of all aspects as reported herein, the multispecific antibody is characterized in that said multispecific antibody is of human subclass IgG1 or human subclass IgG4. In one further embodiment of all aspects as reported herein, the multispecific antibody is characterized in that said multispecific antibody is of human subclass IgG1 with the mutations L234A and L235A (numbering according to Kabat EU index). In one further embodiment of all aspects as reported herein, the multispecific antibody is characterized in that said multispecific antibody is of human subclass IgG1 with the mutations L234A, L235A and P329G (numbering according to Kabat EU index). In one further embodiment of all aspects as reported herein, the multispecific antibody is characterized in that said multispecific antibody is of human subclass IgG4 with the mutations S228P and L235E (numbering according to Kabat EU index). In one further embodiment of all aspects as reported herein, the multispecific antibody is characterized in that said multispecific antibody is of human subclass IgG4 with the mutations S228P, L235E and P329G (numbering according to Kabat EU index).

In one embodiment of all aspects as reported herein, an antibody comprising a heavy chain including a CH3 domain as specified herein, comprises an additional C-terminal glycine-lysine dipeptide (G446 and K447, numbering according to Kabat EU index). In one embodiment of all aspects as reported herein, an antibody comprising a heavy chain including a CH3 domain, as specified herein, comprises an additional C-terminal glycine residue (G446, numbering according to Kabat EU index).

### 6. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions”. More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, P. S., Methods Mol. Biol. 207 (2008) 179-196), and/or residues that contact antigen, with the resulting variant VH or VL being tested for binding affinity. Affinity maturation by constructing and reselecting from secondary libraries has been described, e.g., in Hoogenboom, H. R. et al. in Methods in Molecular Biology 178 (2002) 1-37. In some embodiments of affinity maturation, diversity is introduced into the variable genes chosen for maturation by any of a variety of methods (e.g., errorprone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham, B. C. and Wells, J. A., Science 244 (1989) 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the Nor C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc-region, the carbohydrate attached thereto may be altered. Native antibodies produced by mammalian cells typically comprise a branched, biantennary oligosaccharide that is generally attached by an N-linkage to Asn297 of the CH2 domain of the Fc-region (see, e.g., Wright, A. and Morrison, S. L., TIBTECH 15 (1997) 26-32). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc-region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc-region (EU numbering of Fc-region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki, A. et al., J. Mol. Biol. 336 (2004) 1239-1249; Yamane-Ohnuki, N. et al., Biotech. Bioeng. 87 (2004) 614-622. Examples of cell lines capable of producing defucosylated antibodies include Lec13 CHO cells deficient in protein fucosylation (Ripka, J. et al., Arch. Biochem. Biophys. 249 (1986) 533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki, N. et al., Biotech. Bioeng. 87 (2004) 614-622; Kanda, Y. et al., Biotechnol. Bioeng. 94 (2006) 680-688; and WO 2003/085107).

Antibody variants are further provided with bisected oligosaccharides, e.g., in which a biantennary oligosaccharide attached to the Fc-region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc-region are also provided. Such antibody variants may have improved CDC function. Such antibody variants are described, e.g., in WO 1997/30087; WO 1998/58964; and WO 1999/22764.

### c) Fc-Region Variants

In certain embodiments, one or more amino acid modifications may be introduced into the Fc-region of an antibody provided herein, thereby generating an Fc-region variant. The Fc-region variant may comprise a human Fc-region sequence (e.g., a human IgG1, IgG2, IgG3 or IgG4 Fc-region) comprising an amino acid modification (e.g. a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc [symbol] RIII only, whereas monocytes express FcγRI, FcγRII and FcγRIII. FcR expression on hematopoietic cells is summarized in Table 3 on page 464 of Ravetch, J. V. and Kinet, J. P., Annu. Rev. Immunol. 9 (1991) 457-492. Non-limiting examples of in vitro assays to assess ADCC activity of a molecule of interest is described in U.S. Pat. No. 5,500,362 (see, e.g. Hellstrom, I. et al., Proc. Natl. Acad. Sci. USA 83 (1986) 7059-7063; and Hellstrom, I. et al., Proc. Natl. Acad. Sci. USA 82 (1985) 1499-1502); U.S. Pat. No. 5,821,337 (see Bruggemann, M. et al., J. Exp. Med. 166 (1987) 1351-1361). Alternatively, non-radioactive assays methods may be employed (see, for example, ACTI™ nonradioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, R. et al., Proc. Natl. Acad. Sci. USA 95 (1998) 652-656. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro, H. et al., J. Immunol. Methods 202 (1996) 163-171; Cragg, M. S. et al., Blood 101 (2003) 1045-1052; and Cragg, M. S. and M. J. Glennie, Blood 103 (2004) 2738-2743). FcRn binding and in vivo clearance/half-life determinations can also be performed using methods known in the art (see, e.g., Petkova, S. B. et al., Int. Immunol. 18 (2006) 1759-1769).

Antibodies with reduced effector function include those with substitution of one or more of Fc-region residues 238, 265, 269, 270, 297, 327, and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields, R. L. et al., J. Biol. Chem. 276 (2001) 6591-6604).

In certain embodiments, an antibody variant comprises an Fc-region with one or more amino acid substitutions which improve ADCC, e.g., substitutions at positions 298, 333, and/or 334 of the Fc-region (EU numbering of residues).

In some embodiments, alterations are made in the Fc-region that result in altered (i.e., either improved or diminished) C1q binding and/or Complement Dependent Cytotoxicity (CDC), e.g., as described in U.S. Pat. No. 6,194,551, WO 99/51642, and Idusogie, E. E. et al., J. Immunol. 164 (2000) 4178-4184.

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc-region with one or more substitutions therein which improve binding of the Fc-region to FcRn. Such Fc variants include those with substitutions at one or more of Fc-region residues: 238, 256, 265, 272, 286, 303, 305, 307, 311, 312, 317, 340, 356, 360, 362, 376, 378, 380, 382, 413, 424 or 434, e.g., substitution of Fc-region residue 434 (U.S. Pat. No. 7,371,826).

See also Duncan, A. R. and Winter, G., Nature 322 (1988) 738-740; U.S. Pat. Nos. 5,648,260; 5,624,821; and WO 94/29351 concerning other examples of Fc-region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc-region. Cysteine engineered antibodies may be generated as described, e.g., in U.S. Pat. No. 7,521,541.

### e) Antibody Derivatives

In certain embodiments, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(nvinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or non-branched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an antitransferrin receptor antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-transferrin receptor antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-transferrin receptor antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fe effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P. et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

### C. Assays

Anti-transferrin receptor antibodies provided herein may be identified, screened for, or characterized for their physical/chemical properties and/or biological activities by various assays known in the art.

### 1. Binding Assay

In one aspect, an antibody of the invention is tested for its antigen binding activity, e.g., by known methods such as ELISA, alphaLISA, Western blot, antibody or reverse phase array, etc.

In an exemplary ELISA or alphaLISA assay, transferrin receptor in solution (cell supernatant, cell or tissue lysates, body fluids etc.) is bound by a capture antibody, which specifically binds to a first epitope on the transferrin receptor, or transferrin receptor in a certain conformation and a detection antibody coupled to a detection entity, which specifically binds to a second epitope or conformation of the transferrin receptor. The readout is based on the detection entity (chemiluminescence, fluorescence, energy transfer induced luminescence etc.).

In the case of antibody array, antibodies are spotted onto glass or nitrocellulose chips. The slides are blocked and incubated with transferrin receptor containing solution, washed to remove unbound antibodies and bound antibodies are detected with a fluorescently labeled corresponding secondary antibody. The fluorescence signal is measured by a fluorescence slide scanner. Similarly, for a reverse phase array, recombinant transferrin receptor, cell supernatant, cell or tissue lysates, body fluids etc. are spotted onto glass or nitrocellulose chips. The slides are blocked and individual arrays are incubated with an antibody against a specific epitope on the transferrin receptor. Unbound antibodies are washed off and bound antibodies are detected with a fluorescently labeled corresponding secondary antibody. The fluorescence signal is measured by a fluorescence slide scanner (Dernick, G., et al., J. Lipid Res. 52 (2011) 2323-2331).

### D. Methods and Compositions for Diagnostics and Detection

In certain embodiments, any of the anti-transferrin receptor antibodies provided herein is useful for detecting the presence of human transferrin receptor in a biological sample. The term “detecting” as used herein encompasses quantitative or qualitative detection. In certain embodiments, a biological sample comprises a cell or tissue, such as brain tissue.

In one embodiment, an anti-transferrin receptor antibody for use in a method of diagnosis or detection is provided. In a further aspect, a method of detecting the presence of the transferrin receptor in a biological sample is provided. In certain embodiments, the method comprises contacting the biological sample with an anti-transferrin receptor antibody as described herein under conditions permissive for binding of the antitransferrin receptor antibody to the transferrin receptor, and detecting whether a complex is formed between the anti-transferrin receptor antibody and the transferrin receptor. Such method may be an in vitro or in vivo method. In one embodiment, an antitransferrin receptor antibody is used to select subjects eligible for therapy with an antitransferrin receptor antibody, e.g. where the transferrin receptor is a biomarker for selection of patients.

Exemplary disorders that may be diagnosed using an antibody of the invention include neurodegeneration with brain iron accumulation type 1 (NBIA1), pure autonomic failure, Down's syndrome, complex of Guam, and several Lewy body disorders, such as diffuse Lewy body disease (DLBD), the Lewy body variant of Alzheimer's disease (LBVAD), certain forms of Gaucher's disease, and Parkinson's Disease dementia (PDD).

In certain embodiments, labeled anti-transferrin receptor antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E. Pharmaceutical Formulations

Pharmaceutical formulations of an anti-transferrin receptor antibody as described herein are prepared by mixing such antibody having the desired degree of purity with one or more optional pharmaceutically acceptable carriers (Remington's Pharmaceutical Sciences, 16th edition, Osol, A. (ed.) (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyl dimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as poly(vinylpyrrolidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH20 (HYLENEX®, Baxter International, Inc.). Certain exemplary sHASEGPs and methods of use, including rhuPH20, are described in US 2005/0260186 and US 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences, 16th edition, Osol, A. (ed.) (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### III. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-transferrin receptor antibody.

### VI. EXAMPLES

The following are examples of methods and compositions of the invention. It is understood that various other embodiments may be practiced, given the general description provided above.

### Materials and Methods

### Recombinant DNA Techniques

Standard methods were used to manipulate DNA as described in Sambrook, J. et al., Molecular cloning: A laboratory manual; Cold Spring Harbor Laboratory Press, Cold Spring Harbor, New York, 1989. The molecular biological reagents were used according to the manufacturer's instructions.

### Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (PlaneggMartinsried, Germany).

### Reagents

All commercial chemicals, antibodies and kits were used as provided according to the manufacturer's protocol if not stated otherwise.

### Example 1

### Immunization of Rabbits and Mice

### Immunization of Mice

NMRI mice were immunized genetically, using a plasmid expression vector coding for full-length human or cynomolgus TfR by intradermal application of 100 μg vector DNA, followed by electroporation (2 square pulses of 1000 V/cm, duration 0.1 ms, interval 0.125 s; followed by 4 square pulses of 287.5 V/cm, duration 10 ms, interval 0.125 s. Mice received either 6 or 7 consecutive immunizations at days 0, 14, 28, 42, 56, 70, and 84. The fourth and sixth immunizations were performed with vector coding for cynomolgus TfR; vector coding for human TfR was used for all other immunizations. Blood was taken at days 36, 78 and 92 and serum prepared, which was used for titer determination by ELISA (see below). Animals with highest titers were selected for boosting at day 96, by intravenous injection of either 10⁶ human TF-1 cells or 50 μg of recombinant human soluble TfR lacking the helical domain (extracellular domain of the human TfR beginning at Leu22, ending at Asn608, expressed in HEK293F cells as an N-terminal fusion to human Fc-region and purified by protein A affinity chromatography and size exclusion chromatography, and monoclonal antibodies were isolated by hybridoma technology, based on their ability to bind human and cynomolgus transferrin receptor expressed on the surface of stably transfected CHO-K1 cells (see Example 4).

### Immunization of Rabbits

New Zealand White rabbits or transgenic rabbits expressing a humanized antibody repertoire were immunized genetically, using a plasmid expression vector coding for full-length human or cynomolgus TfR, by intradermal application of 400 μg vector DNA, followed by electroporation (5 square pulses of 750 V/cm, duration 10 ms, interval 1 s.). Rabbits received 6 consecutive immunizations at days 0, 14, 28, 56, 84 and 112. The fourth and sixth immunizations were performed with vector coding for cynomolgus TfR; vector coding for human TfR was used for all other immunizations. Blood (10% of estimated total blood volume) was taken at days 35, 63, 91 and 119.

Serum was prepared, which was used for titer determination by ELISA (see below), and peripheral mononuclear cells were isolated, which were used as a source of antigenspecific B cells in the B cell cloning process (see Example 2).

### Determination of Serum Titers (ELISA)

Human recombinant soluble TfR (R&D Systems Cat. No. 2474-TR) was immobilized on a 96-well NUNC Maxisorb plate at 3 μg/mL, 100 μL/well, in PBS, followed by: blocking of the plate with 2% CroteinC in PBS, 200 μL/well; application of serial dilutions of antisera, in duplicates, in 0.5% CroteinC in PBS, 100 μL/well; detection with (1) HRP-conjugated goat anti-mouse antibody (Jackson Immunoresearch/Dianova 115-036-071; 1/16 000) for all mouse sera, (2) HRP-conjugated donkey anti-rabbit IgG antibody (Jackson Immunoresearch/Dianova 711-036-152; 1/16 000) for all rabbit sera, (3) rabbit anti-human IgG antibody (Pierce/Thermo Scientific 31423; 1/5000) for sera from transgenic rabbits only, (4) biotinylated goat anti-human kappa antibody (Southern Biotech/Biozol 2063-08, 1/5 000) and streptavidin-HRP for sera from transgenic rabbits only; diluted in 0.5% CroteinC in PBS, 100 μL/well. For all steps, plates were incubated for 1 h at 37° C. Between all steps, plates were washed 3 times with 0.05% Tween 20 in PBS. Signal was developed by addition of BM Blue POD Substrate soluble (Roche), 100 μL/well; and stopped by addition of 1 M HCl, 100 μL/well. Absorbance was read out at 450 nm, against 690 nm as reference. Titer was defined as dilution of antisera resulting in half-maximal signal.

### Example 2

B-Cell Cloning from Rabbits

### Isolation of Rabbit Peripheral Blood Mononuclear Cells (PBMC)

Blood samples were taken of in summary 6 animals (2 wild-type (wt) rabbits and 4 transgenic (tg) rabbits). These rabbits derived from 2 different immunization campaigns: first campaign with 2 wt and 2 tg rabbits and second campaign with 2 tg rabbits (see also the example “Immunization of rabbits”). EDTA containing whole blood was diluted twofold with 1×PBS (PAA, Pasching, Austria) before density centrifugation using lympholyte mammal (Cedarlane Laboratories, Burlington, Ontario, Canada) according to the specifications of the manufacturer. The PBMCs were washed twice with 1×PBS.

EL-4 B5 medium

RPMI 1640 (Pan Biotech, Aidenbach, Germany) supplemented with 10% FCS (Hyclone, Logan, UT, USA), 2 mM glutamine, 1% penicillin/streptomycin solution (PAA, Pasching, Austria), 2 mM sodium pyruvate, 10 mM HEPES (PAN Biotech, Aidenbach, Germany) and 0.05 mM P-mercaptoethanol (Gibco, Paisley, Scotland)

### Depletion of Cells

First immunization campaign: Sterile 6-well plates (cell culture grade) covered with a confluent monolayer of CHO cells were used to deplete macrophages/monocytes through unspecific adhesion as well as non-specifically binding lymphocytes.

Second immunization campaign: The depletion step using wells covered with CHO cells was omitted since we could not exclude those B-cells producing antibodies that are cross-reactive to hamster transferrin receptor antibodies would be depleted. Therefore, blank sterile 6-well plates (cell culture grade) were used to deplete macrophages and monocytes through unspecific adhesion enabling potential B-lymphocytes producing hamster cross-reactive (and possibly mouse cross-reactive) surface antibodies to reach the next step in the workflow.

For each immunization campaign: each well was filled at maximum with 4 mL medium and up to 6×10⁶ PBMCs from the immunized rabbit and allowed to bind for 1 h at 37° C. in the incubator. The cells in the supernatant (peripheral blood lymphocytes (PBLs)) were used for the antigen panning step.

### Enrichment of B-Cells on the Human Transferrin Receptor

6-well tissue culture plates covered with a monolayer of human transferrin receptorpositive CHO cells were seeded with up to 6×10⁶ PBLs per 4 mL medium and allowed to bind for 1 h at 37° C. in the incubator. Non-adherent cells were removed by carefully washing the wells 1-2 times with 1×PBS. The remaining sticky cells were detached by trypsin for 10 min. at 37° C. in the incubator. Trypsination was stopped with EL-4 B5 medium. The cells were kept on ice until the immune fluorescence staining.

### Immune Fluorescence Staining and Flow Cytometry

The anti-IgG FITC (AbD Serotec, Dusseldorf, Germany) was used for single cell sorting. For surface staining, cells from the depletion and enrichment step were incubated with the anti-IgG FITC antibody in PBS and incubated for 45 min. in the dark at 4° C. After staining the PBMCs were washed two fold with ice cold PBS. Finally, the PBMCs were resuspended in ice cold PBS and immediately subjected to the FACS analyses. Propidium iodide in a concentration of 5 μg/mL (BD Pharmingen, San Diego, CA, USA) was added prior to the FACS analyses to discriminate between dead and live cells.

A Becton Dickinson FACSAria equipped with a computer and the FACSDiva software (BD Biosciences, USA) were used for single cell sort.

### B-Cell Cultivation

The cultivation of the rabbit B-cells was prepared by a method similar to that described by Zubler et al. (1985). Briefly, single sorted rabbit B-cells were incubated in 96-well plates with 200 μL/well EL-4 B5 medium containing Pansorbin Cells (1:100000) (Calbiochem (Merck), Darmstadt, Deutschland), 5% rabbit thymocyte supernatant (charge TSN-M13 (10242), MicroCoat, Bernried, Germany) and gamma-irradiated murine EL-4-B5 thymoma cells (2.5×10⁴/well) for 7 days at 37° C. in an atmosphere of 5% CO₂ in the incubator. The supernatants of the B-cell cultivation were removed for screening and the remaining cells were harvested immediately and were frozen at −80° C. in 100 L RLT buffer (Qiagen, Hilden, Germany).

### Example 3

### Phage Display for Selection and Production of Monovalent Anti-TfR IgGs

### Selection by Phage Display

Generation of antibodies binding to human and cynomolgus TfR was carried out by phage display using standard protocols (Silacci et al, Proteomics, 5 (2005) 2340-2350). A synthesized gene for hTfR-Fc(KiH)-Avi (KiH=knobs-into-holes, Avi=AviTag) antigen was cloned by connecting the hTfR ECD to the N-terminal hinge of a human hole Fc-region, which carried a C-terminal Avi-tag (SEQ ID NO: 99), and ligation into a mammalian expression vector. All our mammalian expression vectors carry a MPSV promoter for initiation of transcription and translation, where transcription is terminated by a synthetic polyA signal sequence located downstream of the ORF. In addition, the vector contains an EBV oriP sequence for autonomous replication in EBV-EBNA expressing cell lines. The correct ORF was verified by sequencing. This vector was used for expression in HEK293 EBNA suspension cells, by co-expression with an empty knob-Fc-region and the BirA protein, and adding 1 mM of biotin to the culture medium. The resulting biotinylated protein was purified by protein A affinity chromatography, followed by size exclusion chromatography. The cyTfR-Fc(KiH)-Avi antigen was produced respectively (SEQ ID NO: 100).

The library used was a fully synthetic library using human frameworks of the VH1, 3, 4, and 5 families, as well as V-kappa1, -2, and -3, and V-lambda3. Randomized aminoacids were in CDR-H3 and CDR-L3. Library pools were generated, using each heavy chain VH together with all different light chain libraries. Selections were carried out in solution according to the following procedure: 1. binding of approx. 10¹² phageimid particles of each library to 100 nM biotinylated TfR-avi-his for 0.5 h in a total volume of 1 mL, 2. capture of biotinylated TfR-avi-his and specifically bound phage particles by addition of 5.4×10⁷ streptavidin-coated magnetic beads for 10 min., 3. washing of beads using 5-10×1 mL PBS/Tween 20 and 5-10×1 mL PBS, 4. elution of phage particles by addition of 1 mL 100 mM TEA (triethylamine) for 10 min. and neutralization by adding 500 L 1 M Tris/HCl pH 7.4 and 5. Re-infection of exponentially growing E. coli TG1 bacteria, infection with helper phage VCSM13 and subsequent PEG/NaCl precipitation of phageimid particles to be used in subsequent selection rounds. Selections were carried out over 3-5 rounds using either constant or decreasing (from 10⁻⁷ M to 2×10⁻⁹ M) antigen concentrations. In round 2, capture of antigen/phage complexes was performed using neutravidin plates instead of streptavidin beads. Since the binders generated against the recombinant soluble antigen often showed only weak binding on cells, we introduced two additional selection steps using cells displaying the native antigen after the second or third round of enrichment on the recombinant antigen. Here, the two cell-lines TF-1, and NCI-H460 were used (both available from ATCC). In brief, 1*10⁶ cells were incubated with approx. 10¹² phage particles for 1h on ice to avoid receptor mediated internalization. Washing was performed by 5-10 centrifugation steps using PBST buffer (PBS containing 1% Tween20). Entire cells were used to infect the TG1 bacteria for phage rescue.

Specific binders were identified by ELISA as follows: 100 L of 10 nM biotinylated TfR-avi-his per well were coated on neutravidin plates. Fab-containing bacterial supernatants were added and binding Fabs were detected via their FLAG-tags by using an anti-FLAG/HRP secondary antibody. ELISA-positive clones were bacterially expressed as soluble Fab fragments in 96-well format and supernatants were subjected to a kinetic screening experiment by SPR-analysis using ProteOn XPR36 (BioRad).

Clones expressing Fabs with the highest affinity constants were identified and the corresponding phageimids were sequenced.

### Purification of Fab Antibodies

Top10 cells were individually transfected with the phageimid plasmid of all cell ELISA positive clones. The cells were grown in media and production of Fab antibodies was induced. Fab antibodies were isolated by periplasmic preparation and purified using IMAC.

### FACS Analysis

Purified Fab antibodies were applied to TF-1 cells in various concentrations. Cell bound Fab antibodies were detected using a fluorescent labeled anti-Fab antibody and measurements by FACS. EC50 values were calculated.

### Production and Characterization of the Selected Clones

Conversion into the IgG Format

Selected clones with a complex half live (t½) between 6 to 20 minutes or an EC₅₀ on cells between 10 nM and 500 nM, with a distinct cell binding signal either on FACS or cell ELISA, and cross-reactive binding against both the hTfR-Fc(KiH)-Avi and the cyTfR-Fc(KiH)-Avi antigen were chosen for conversion into the IgG format.

Therefore, the VL domain was amplified by PCR and cloned into a mammalian expression vector, as described above, directly upstream of a CL domain. In addition, the VH domain was amplified by PCR and cloned directly upstream of a CH-Fc domain. The sequences of both expression vectors were determined.

### Production of IgG Antibodies

HEK293 EBNA suspension cells were transfected with both, the LC and HC encoding, plasmids and cultivated for 7 days. The supernatant was cleared by sterile filtration. IgG concentration was determined by protein A chromatography.

### Determination of EC50 Using the TagLite Technology

The gene of the ECD of hTfR or cyTfR, respectively, including the transmembrane domain was ligated downstream to SNAP-tag into a mammalian expression vector. This vector was used to transfect HEK293 EBNA suspension cells, resulting in display of the TfR with a C-terminal SNAP tag fusion ((SEQ ID NO: 101, SEQ ID NO: 102). The SNAP tag was specifically labeled with SNAP-Lumi4Tb (Cisbio). The labeling efficiency determined by measuring the emission of Terbium at 615 nm. Labeled cells were stored at −80° C.

In presence of anti-humanFc-d2 IgG antibody, labeled cells were incubated with IgG supernatant in various dilutions and the FRET signals (emission of donor dye Lumi4Tb: 615 nM and acceptor dye d2: 665 nM) were measured after 4 hours. The EC₅₀ values were calculated, resulting in 25 IgGs, which bound to both hTfR and cyTfR.

### Determination of Cell Binding by FACS

The gene of full-length hTfR or cyTfR, respectively, was cloned into a mammalian expression vector. This vector was used for transfection of CHO EBNA suspension cells. IgG supernatant was directly applied to the TfR displaying cells. After washing with PBST, the antigen-antibody complex was detected using an anti-huFc IgG-HRP antibody conjugate, followed by development with 3,3′-5,5′-Tetramethylbenzidine. Functional display was verified using commercially available anti-TfR antibody. All 25 TagLite positive clones exhibited a strong binding signal.

### Production and Purification of Monovalent IgG Like Molecules

The VH domain was cloned into a mammalian expression vector encoding the human knob IgG1 HC, which carries the L234A, L235A, and P329G mutations. This vector was used for expression in HEK293 EBNA suspension cells, co-expressing the LC (vector described above) and an empty hole-Fc domain carrying the L234A, L235A, P329G, H435R and Y436F mutations. The resulting protein was purified by protein A affinity chromatography. In cases of monomeric protein was >95%, determined by analytical size exclusion chromatography, the protein was further purified by size exclusion chromatography.

### Example 4

### Identification of Human and Cynomolgus TfR-Binding Antibodies by Cell ELISA

To screen rabbit B-cell or mouse hybridoma supernatants for antibodies recognizing human and cynomolgus TfR, a cell ELISA using stably transfected CHO-K1 cells way employed. Stable transfectants were obtained by transfecting CHO-K1 cells with expression plasmids containing expression cassettes for the human or cynomolgus TfR as well as for neomycin-phosphotransferase. After transfection, cells were diluted in growth medium containing 500 μg/mL G418 (Life Technologies). After appearance of growing clones, cells were detached, stained with MEM-75 (Abcam) or 13E4 (Life Technologies) and PE-labeled secondary antibodies for human or cynomolgus TfR, and highly fluorescent cells sorted as single cells into 96-well-plate wells (FACS Aria). After 7 days of growth, clones were again checked for TfR expression and best expressing clones selected for cell ELISA experiments.

Briefly, 15,000 cells were seeded per well of a 384-well plate and incubated for 18 h at 37° C., 5% CO₂. Supernatant was removed using an automated washer (BIOTEK), and 30 μL of antibody-containing supernatant added to each well, followed by 24 μL of growth medium. After 2 hours of incubation, wells were emptied and 30 μL of 0.05% glutaraldehyde in PBS added for 45 min. at RT. After 3 washes with PBS/0.025% Tween20 (PBST), 30 μL of anti-rabbit-HRP or anti-mouse-HRP (Southern Biotech) diluted 1:5000 in Blocking buffer was added and plates incubated for 1 hour at RT. Wells were washed 6 times with PBST and signal was generated using 30 μL of TMB per well and absorbance measured at 450 nm.

### Example 5

### Cloning and Expression of Anti-TfR Antibodies

### Recombinant DNA Techniques

Standard methods were used to manipulate DNA as described in Sambrook, J. et al., Molecular cloning: A laboratory manual; Cold Spring Harbor Laboratory Press, Cold Spring Harbor, New York, 1989. The molecular biological reagents were used according to the manufacturer's instructions.

### Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (PlaneggMartinsried, Germany).

PCR amplification of V-domains

Total RNA was prepared from B-cells lysate (resuspended in RLT buffer—Qiagen—Cat. N° 79216) using the NucleoSpin 8/96 RNA kit (Macherey & Nagel; 740709.4, 740698) according to manufacturer's protocol. RNA was eluted with 60 μL RNAse free water. 6 μL of RNA was used to generate cDNA by reverse transcriptase reaction using the Superscript III First-Strand Synthesis SuperMix (Invitrogen 18080-400) and an oligodT-primer according to the manufacturer's instructions. All steps were performed on a Hamilton ML Star System. 4 μL of cDNA were used to amplify the immunoglobulin heavy and light chain variable regions (VH and VL) with the AccuPrime SuperMix (Invitrogen 12344-040) in a final volume of 50 μL using the primers rbHC.up and rbHC.do for the heavy chain, rbLC.up and rbLC.do for the light chain of Wild Type Rabbit B cells and BcPCR_FHLC_leader.fw and BcPCR_huCkappa.rev for the light chain of transgenic rabbit B-cells (see Table below). All forward primers were specific for the signal peptide (of respectively VH and VL) whereas the reverse primers were specific for the constant regions (of respectively VH and VL). The PCR conditions for the RbVH+RbVL were as follows: Hot start at 94° C. for 5 min.; 35 cycles of 20 sec. at 94° C., 20 sec. at 70° C., 45 sec. at 68° C., and a final extension at 68° C. for 7 min. The PCR conditions for the HuVL were as follows: Hot start at 94° C. for 5 min.; 40 cycles of 20 sec. at 94° C., 20 sec. at 52° C., 45 sec. at 68° C., and a final extension at 68° C. for 7 min.

| | |
|---|---|
| rbHC.up | AAGCTTGCCACCATGGAGACTGGGCT |
| (SEQ ID NO: 103) | GCGCTGGCTTC |
| rbHCf.do | CCATTGGTGAGGGTGCCCGAG |
| (SEQ ID NO: 104) | |
| rbLC.up | AAGCTTGCCACCATGGACAYGAGGGC |
| (SEQ ID NO: 105) | CCCCACTC |
| rbLC.do | CAGAGTRCTGCTGAGGTTGTAGGTAC |
| (SEQ ID NO: 106) | |
| BcPCR_FHLC_leader.fw | ATGGACATGAGGGTCCCCGC |
| (SEQ ID NO: 107) | |
| BcPCR_huCkappa.rev | GATTTCAACTGCTCATCAGATGGC |
| (SEQ ID NO: 108) | |

8 μL of 50 μL PCR solution were loaded on a 48 E-Gel 2% (Invitrogen G8008-02). Positive PCR reactions were cleaned using the NucleoSpin Extract II kit (Macherey & Nagel; 740609250) according to manufacturer's protocol and eluted in 50 μL elution buffer. All cleaning steps were performed on a Hamilton ML Starlet System.

### Recombinant Expression of Rabbit Monoclonal Bivalent Antibodies

For recombinant expression of rabbit monoclonal bivalent antibodies, PCR-products coding for VH or VL were cloned as cDNA into expression vectors by the overhang cloning method (R S Haun et al., BioTechniques (1992) 13, 515-518; M Z Li et al., Nature Methods (2007) 4, 251-256). The expression vectors contained an expression cassette consisting of a 5′ CMV promoter including intron A, and a 3′ BGH poly adenylation sequence. In addition to the expression cassette, the plasmids contained a pUC18-derived origin of replication and a beta-lactamase gene conferring ampicillin resistance for plasmid amplification in E. coli. Three variants of the basic plasmid were used: one plasmid containing the rabbit IgG constant region designed to accept the VH regions while two additional plasmids containing rabbit or human kappa LC constant region to accept the VL regions.

Linearized expression plasmids coding for the kappa or gamma constant region and VL/VH inserts were amplified by PCR using overlapping primers.

Purified PCR products were incubated with T4 DNA-polymerase which generated single-strand overhangs. The reaction was stopped by dCTP addition.

In the next step, plasmid and insert were combined and incubated with recA which induced site specific recombination. The recombined plasmids were transformed into E. coli. The next day the grown colonies were picked and tested for correct recombined plasmid by plasmid preparation, restriction analysis and DNA-sequencing.

For antibody expression, the isolated HC and LC plasmids were transiently cotransfected into HEK293 cells and the supernatants were harvested after 1 week.

### Generation of Vectors for the Expression of Rabbit Monoclonal Monovalent Antibodies

For recombinant expression of selected candidates as monoclonal monovalent antibodies rabbit constant regions of all VH chains were converted into human constant regions enclosing the knob-mutation in the CH3 segment. For VL chains derived from rabbit wild-type B-cells, rabbit C kappa constant regions were converted into human. 4 μL of cDNA of the selected candidates were used to amplify the immunoglobulin heavy and light chain variable regions with the AccuPrime SuperMix (Invitrogen 12344-040) in a final volume of 50 μL with forward primers specific for the signal peptide and reverse primers specific for the CDR3-J region with (at the 3′ end) overlap sequence (20 bp) homologous to the human constant regions (respectively of VH and VL). The PCR conditions for the VH and VL chain amplification were as follows: Hot start at 94° C. for 5 min.; 35 cycles of 20 sec. at 94° C., 20 sec. at 68° C., 45 sec. at 68° C., and a final extension at 68° C. for 7 min.

PCR-products coding for VH or VL were cloned as cDNA into expression vectors by the overhang cloning method (RS Haun et al., BioTechniques (1992) 13, 515-518; M Z Li et al., Nature Methods (2007) 4, 251-256). The expression vectors contained an expression cassette consisting of a 5′ CMV promoter including intron A, and a 3′ BGH poly adenylation sequence. In addition to the expression cassette, the plasmids contained a pUC18-derived origin of replication and a beta-lactamase gene conferring ampicillin resistance for plasmid amplification in E. coli. Two variants of the basic plasmid were used: one plasmid containing the human IgG constant region designed to accept the new amplified VH chain and a second plasmid containing the human kappa LC constant region to accept the VL chain.

Linearized expression plasmids coding for the kappa or gamma constant region and VL/VH inserts were amplified by PCR using overlapping primers.

Purified PCR products were incubated with T4 DNA-polymerase which generated single-strand overhangs. The reaction was stopped by dCTP addition.

In the next step, plasmid and insert were combined and incubated with recA which induced site specific recombination. The recombined plasmids were transformed into E. coli. The next day the grown colonies were picked and tested for correct recombined plasmid by plasmid preparation, restriction analysis and DNA-sequencing.

### Example 6

### Transient Expression of the Monovalent Anti-TfR Antibodies

The antibodies were generated in vivo in transiently transfected HEK293 cells (human embryonic kidney cell line 293-derived) cultivated in F17 Medium (Invitrogen Corp.).

For transfection “293-Free” Transfection Reagent (Novagen) was used. Antibodies and antibody-based modified molecules as described above were expressed from individual expression plasmids. Transfections were performed as specified in the manufacturer's instructions. Recombinant protein-containing cell culture supernatants were harvested three to seven days after transfection. Supernatants were stored at reduced temperature (e.g. −80° C.) until purification.

General information regarding the recombinant expression of human immunoglobulins in e.g. HEK293 cells is given in: Meissner, P. et al., Biotechnol. Bioeng. 75 (2001) 197-203.

### Example 7

### Purification of One-Armed Transferrin Receptor Antibodies in High Throughput

The 50 mL clarified supernatants containing one armed antibodies in 96 deep-well plates were loaded on 200 μL MabSelectSuRe columns. After washing steps with PBS at pH 7.4, proteins were eluted with 2.5 mM HCl using Tecan/Atoll-system resulting in 0.5 mL eluate. Eluate was neutralized by 2 M Tris pH 8. Purified proteins were quantified using a Nanodrop spectrophotometer and analyzed by CE-SDS under denaturing and reducing conditions and analytical SEC. To obtain protein with high purity (>95%) a large proportion of the antibodies have to be purified further on size exclusion chromatography to separate from half antibody, knob-knob antibodies and higher aggregates. In the following 500 μL of the samples were injected on Superdex200 10/300GL in 20 mM histidine containing 140 mM NaCl pH 6.0 using Dionex UltiMate 3000. This method allows fractionating 25-30 samples/day and therefore allows polishing a large number of screening hits in one-armed format. Fractions were pooled and analyzed again as described above.

### Example 8

hCMEC/D3 Cell Culture for Transcytosis Assays

Medium and supplements for hCMEC/D3 (Weksler, B. B. et al., FASEB J. 19 (2005), 1872-1874) were obtained from Lonza. hCMEC/D3 cells (passages 26-29) were cultured to confluence on collagen-coated coverslips (microscopy) or flasks in EBM2 medium containing 2.5% FBS, a quarter of the supplied growth factors and fully complemented with supplied hydrocortisone, gentamycin and ascorbic acid.

For all transcytosis assays, high density pore (1×10⁸ pores/cm²) PET membrane filter inserts (0.4 μm, 12 mm diameter) were used in 12-well cell culture plates. Optimum media volumes were calculated to be 400 μL and 1600 μL for apical and basolateral chambers, respectively. Apical chambers of filter inserts were coated with rat tail collagen I (7.5 μg/cm²) followed by fibronectin (5 μg/mL), each incubation lasting for 1 hour at RT. hCMEC/D3 cells were grown to confluent monolayers (approx. 2×10⁵ cells/cm²) for 10-12 days in EMB2 medium.

### Example 9

### Transcytosis Assay of Monovalent Antibodies

The entire assay was performed in serum-free EBM2 medium which was otherwise reconstituted as described in Example 1. Filter inserts with cells were incubated apically with monovalent antibodies (concentration: 2.67 μg/mL) for 1 hour at 37° C. following which the entire apical and basolateral media were collected. From these values, paracellular flux was calculated. The monolayers were washed at RT in serum-free medium apically (400 μL) and basolaterally (1600 μL) 3×3-5 min. each. All the washes were collected to monitor efficiency of removal of the unbound antibody. Pre-warmed medium was added to the apical chamber and the filters transferred to a fresh 12 well plate (blocked overnight with PBS containing 1% BSA) containing 1600 μL pre-warmed medium. At this point, cells on filters were lysed in 500 μL RIPA buffer in order to determine specific antibody uptake. The remaining filters were incubated at 37° C. and samples collected at various time points to determine apical and/or basolateral release of antibody. The content of antibody in the samples was quantified using a highly sensitive IgG ELISA (see Example 3). For each time point, data were generated from three filter cell cultures.

### Example 10

Sensitive IgG ELISA after Transcytosis Assay

The entire procedure was performed at RT using an automated washer for the wash steps. A 384-well plate was coated with 30 μL/well of 1 μg/mL anti-human/mouse-IgG, Fcγ-specific in PBS for 2 hours followed by 1 hour incubation in blocking buffer PBS containing 1% BSA or 1% CroteinC for human and mouse IgG assays, respectively). Serially diluted samples from the transcytosis assay and standard concentrations of the antibody used in the transcytosis assay were added to the plate and incubated for 2 hours. After four washes, 30 μL/well of 50 ng/mL anti-human/mouse-F(ab)2-Biotin in blocking buffer was added and incubated for a further 2 hours. Following 6 washes, 30 μL/well of 50 ng/mL (huIgG assay) or 100 ng/mL (mIgG assay) Poly-HRP40-Streptavidin (Fitzgerald; in PBS containing 1% BSA and 0.05% Tween-20) was added and incubated for 30 min. After 4 washes, immune complexes were detected by addition of 30 μL/well of BM Chemiluminescence Substrate (Roche). The luminescence signal was measured using a luminescence plate reader and concentration calculated using the fitted standard curve. The sensitivity of the assay ranged from 10 μg/mL to 10 ng/mL.

### Example 11

Epitope Mapping by Cell ELISA of CHO Cells Transfected with hTfR Mutants

In order to be able determine the epitope regions on human transferrin receptor (hTfR), mutations were introduced into the hTfR sequence at positions where a cluster of surface-exposed amino acids had different amino acids in the aligned mouse TfR sequence (see Table below), following the rationale that in spite of the significant homology between human and mouse TfR (77% identity), no antibodies directed to the extracellular part are known which show good cross-reactivity between both orthologous. Cloning of plasmids with the corresponding mutations is described above. To map binding of human TfR binders to those epitopes, CHO-K1 cells were transiently transfected with the described plasmids and antibody binding measured in a cell ELISA. Briefly, 10⁴ cells were plated per well of a 96-well plate the day before experiment in normal growth medium (RPMI/10% FCS). The other day, medium was changed to OPTI-MEM Serum-Reduced Medium (Gibco), and 10 μL of a mixture of 1200 μL OPTI-MEM, 12 μg plasmid DNA and 12 μL XtremeGENE transfection reagent (Roche) were added to the wells after 30 minutes of pre-incubation. Cells were incubated for 2 days at 37° C./7.5% CO₂, then medium was removed and TfR antibodies added at concentrations between 1 nM and 100 nM in growth medium, followed by 2 h incubation at 4° C. Afterwards, antibody solutions were replaced by 0.05% glutaraldehyde in PBS and cells fixed for 15 min. at RT, then washed twice with PBS and incubated with HRP-conjugated anti-human-Fc secondary antibody (BioRad; 1:2000 in ELISA Blocking Reagent (Roche)) for 1.5 hours at RT. Signal was generated after 3 washes with PBS using 50 μL of TMB per well and absorbance measured at 450 nm.

| Plasmid # | mutations in hTfR |
|---|---|
| 10188 | — |
| 18909 | Thr518Asp/Gln520Lys/Phe521Ser/Gln524Arg |
| 18910 | Arg325Gln |
| 18911 | Ser355Ala/Asp356Arg/Lys358Asn/Thr359Ile |
| 18912 | Asp204Gln/Lys205Ser/Arg208Asn |
| 18913 | Lys574Gly/Glu575Ala/Ile577Thr/Glu578Gln |
| 18914 | Ala196Ile/Gln197Gly/Asn198Gln/Ser199Asn/ |
| | Val200Met/Ile201Val/Ile202Thr/Val203Ile/ |
| | Asp204Val/Lys205Gln/Asn206Ser/Gly207Asn/ |
| | Arg208Gly/Leu209Asn/Val210Leu/Tyr211Asp/ |
| | Leu212Pro |
| 18974 | Asp245Glu/Tyr247Ser/Thr248Tyr/Pro249Ser |

### Example 12

### Surface Plasmon Resonance-Based Binding Assay for Human TfR-Antibody Interaction

The binding experiment were carried out on a BIAcore B 4000 (GE Healthcare) equipped with C1 sensorchip (GE Healthcare, cat. no. BR1005-35) pre-treated with anti-human Fab antibody (GE Healthcare, cat. no 28-9583-25) using a standard amine coupling chemistry procedure accordingly to the vendor's manual.

For kinetic measurements the sample antibody was immobilized applying a contact time of 60 seconds and a flow rate of 10 μL/min in phosphate buffer saline pH 7.4, 0.05% Tween 20 at 25° C. Recombinant His6-tagged human transferrin receptor (R&D systems, cat. no 2474-TR-050) was applied in increasing concentrations and the signal monitored over the time. An average time span of 150 seconds of association time and 600 seconds of dissociation time at 30 μL/min flow rate was recorded. Data were fit using a 1:1 binding model (Langmuir isotherm).

1. A blood brain barrier shuttle module comprising a brain effector entity, optionally a linker and exactly one (monovalent) binding entity which binds to a blood brain barrier receptor, wherein the linker if present couples the effector entity to the monovalent binding entity, which binds to the blood brain barrier receptor, wherein the (monovalent) binding entity, which binds to a blood brain barrier receptor, does not comprise the variable domains of the anti-transferrin receptor antibody 8D3 (SEQ ID NO: 01 and SEQ ID NO: 02) or of the variant anti-transferrin receptor antibody 8D3v (SEQ ID NO: 01 and SEQ ID NO: 03).

2. The blood brain barrier shuttle module according to claim 1, wherein the monovalent binding entity, which binds to the blood brain barrier receptor is a polypeptide.

3. The blood brain barrier shuttle module according to any one of claims 1 to 2, wherein the monovalent binding entity, which binds to the blood brain barrier receptor, comprises a molecule selected from the group consisting of a blood brain barrier receptor ligand, a full length antibody, a scFv, an Fv, a scFab, and a VHH.

4. The blood brain barrier shuttle module according to any one of claims 1 to 3, wherein the blood brain barrier receptor is selected from the group consisting of transferrin receptor, insulin receptor, insulin-like growth factor receptor, low density lipoprotein receptor-related protein 8, low density lipoprotein receptor-related protein 1 and heparin-binding epidermal growth factor-like growth factor.

5. The blood brain barrier shuttle module according to any one of claims 1 to 4, wherein the monovalent binding entity specifically binds to human transferrin receptor and to cynomolgus transferrin receptor.

6. The blood brain barrier shuttle module according to any one of claims 1 to 5, wherein the monovalent binding entity, which binds to the blood brain barrier receptor, comprises one scFab directed to the transferrin receptor.

7. The blood brain barrier shuttle module according to any one of claims 1 to 5, wherein the monovalent binding entity, which binds to the blood brain barrier receptor, comprises one scFv directed to the transferrin receptor.

8. The blood brain barrier shuttle module according to any one of claims 1 to 7, wherein the monovalent binding entity comprises
(i) (a) a HVR-H1 comprising the amino acid sequence of SEQ ID NO: 109; (b) a HVR-H2 comprising the amino acid sequence of SEQ ID NO: 110; (c) a HVR-H3 comprising the amino acid sequence of SEQ ID NO: 112; (d) a HVR-L1 comprising the amino acid sequence of SEQ ID NO: 113; (e) a HVR-L2 comprising the amino acid sequence of SEQ ID NO: 114; and (f) a HVR-L3 comprising the amino acid sequence of SEQ ID NO: 115,
or
(ii) (a) a HVR-H1 comprising the amino acid sequence of SEQ ID NO: 116; (b) a HVR-H2 comprising the amino acid sequence of SEQ ID NO: 117; (c) a HVR-H3 comprising the amino acid sequence of SEQ ID NO: 119; (d) a HVR-L1 comprising the amino acid sequence of SEQ ID NO: 120; (e) a HVR-L2 comprising the amino acid sequence of SEQ ID NO: 121; and (f) a HVR-L3 comprising the amino acid sequence of SEQ ID NO: 122.

9. The blood brain barrier shuttle module according to any one of claims 1 to 8, wherein the brain effector entity is selected from the group consisting of neurological disorder drugs, neurotrophic factors, growth factors, enzymes, cytotoxic agents, antibodies directed to a brain target, monoclonal antibodies directed to a brain target, peptides directed to a brain target.

10. The blood brain barrier shuttle module according to any one of claims 1 to 9, wherein the brain target is selected from the group consisting of β-secretase 1, Aβ (Abeta), epidermal growth factor, epidermal growth factor receptor 2, tau, phosphorylated tau, phosphorylated tau(pS422), apolipoprotein E4, alpha synuclein, oligomeric fragments of alpha synuclein, CD20, huntingtin, prion protein, leucine rich repeat kinase 2, parkin, presenilin 2, gamma secretase, death receptor 6, amyloid precursor protein, p75 neurotrophin receptor and caspase 6.

11. The blood brain barrier shuttle module according to any one of claims 1 to 10, wherein the monovalent binding entity, which binds to the blood brain receptor, is a polypeptide and the monovalent binding entity is conjugated to the C-terminal end of the brain effector entity either directly or via a linker.

12. The blood brain barrier shuttle module according to any one of claims 1 to 11, wherein the brain effector entity comprises a full length antibody directed to a brain target.

13. A pharmaceutical formulation comprising a blood brain barrier shuttle module according to any one of claims 1 to 12.

14. Use of a blood brain barrier shuttle module according to any one of claims 1 to 12 as a medicament.

15. The use according to claim 14, wherein the medicament is for the treatment of a neurological disorder.

16. The blood brain barrier shuttle module according to any one of claims 1 to 12 for use to transport the brain effector entity across the blood brain barrier.


SEQUENCE LISTING (122 sequences)

SEQ ID NO: 1 - protein, 118 aa, Mus musculus
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 2 - protein, 107 aa, Mus musculus
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 3 - protein, 107 aa, synthetic construct
  REGION 1..107: note = variant anti-transferrin receptor antibody 8D3v has a light chain variable domain with mutants L104V and L106I
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIK

SEQ ID NO: 4 - protein, 15 aa, synthetic construct
  REGION 1..15: note = Epitope mapping peptide
IGQNMVTIVQSNGNL

SEQ ID NO: 5 - protein, 15 aa, synthetic construct
  REGION 1..15: note = Epitope mapping peptide
NMVTIVQSNGNLDPV

SEQ ID NO: 6 - protein, 15 aa, synthetic construct
  REGION 1..15: note = Epitope mapping peptide
QSNGNLDPVESPEGY

SEQ ID NO: 7 - protein, 32 aa, synthetic construct
  REGION 1..32: note = Peptide linker
GGGGSGGGGSGGGGSGGGGSGGGGSGGGGSGG

SEQ ID NO: 8 - protein, 20 aa, synthetic construct
  REGION 1..20: note = Peptide linker
GGGGSGGGGSGGGGSGGGGS

SEQ ID NO: 9 - protein, 18 aa, synthetic construct
  REGION 1..18: note = peptidic linker
GGSGGGGSGGGGSGGGGS

SEQ ID NO: 10 - protein, 30 aa, synthetic construct
  REGION 1..30: note = peptidic linker
GGGGSGGGGSGGGGSGGGGSGGGGSGGGGS

SEQ ID NO: 11 - protein, 137 aa, Mus musculus
MEWSWVMLFLLSGTAGVRSEVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 12 - protein, 128 aa, Mus musculus
MDFQVQIFSFLLISASVILSRGQIVLTQSPAIMSASPGEKVTMTCSASSSIDYIHWYQQKSGTSPKRWIYDTSKLASGVPARFSGSGSGTSYSLTISSMEPEDAATYYCHQRNSYPWTFGGGTRLEIR

SEQ ID NO: 13 - protein, 116 aa, synthetic construct
  REGION 1..116: note = clone 0128 VH
QSVEESGGRLVTPGTPLTLTCTVSGIDLSSYYMSWVRQAPGKGLEWIGIIYPSGYAYYTSWAKGRFTISKTSTTVDLKMTSLTTEDTATYFCARDVGDGGGTFDIWGQGTMVTVSS

SEQ ID NO: 14 - protein, 108 aa, synthetic construct
  REGION 1..108: note = clone 128 VL
DIQMTQSPSSLSASVGDRVTITCRASQSISNYLNWYQQKPGKAPKLLIYDASSLESGVPSRFSGSGSGTDFTLTISSLQPEDFATYYCQQSGSTPYTFGQGTKLEIKR

SEQ ID NO: 15 - protein, 121 aa, synthetic construct
  REGION 1..121: note = clone 0143 VH
GVQCQSLEESGGRLVTPGGSLTLTCTVSGIDLSTYSMGWVRQSPGKGLEYIGIISSSDNTYYTSWAKGRFTISKTSTTVDLKITSPTTEDTATYFCARGYNGGWKTPMDVWGQRTTVTVSS

SEQ ID NO: 16 - protein, 108 aa, synthetic construct
  REGION 1..108: note = clone 0143 VL
DIQMTQSPSSLSVSVGDRVTITCRASQGISNYLAWYQQKPGKAPELLIYAASILQGGVPSRFSGSGSGTDFTLTISSLQPEDVATYYCQQYNTYPPTFGQGTKLEIKR

SEQ ID NO: 17 - protein, 129 aa, synthetic construct
  REGION 1..129: note = clone 0146 VH
GVQCEVQLVESGGGLVQPGGSLRLSCAASGFTFGDFAMNWVRQAPGKGLEWVSGISASGVSTYYADSVKGRVTISRDNSKNTLNLQMNSLRAEDTAVYYCARDPCDASNDYGYCKLDVWGQGTTVTVSS

SEQ ID NO: 18 - protein, 108 aa, synthetic construct
  REGION 1..108: note = clone 0146 VL
DIQMTQSPSSLSASVGDRVTITCRASQSISSYLNWYQQKPGKAPKLLIYSASSLQSGVPSRFSGSRSGTEFTLTISSLQPEDFATYYCQQSYNTPLTFGGGTKVEIKR

SEQ ID NO: 19 - protein, 118 aa, synthetic construct
  REGION 1..118: note = clone 0279 VH
QSVEESGGRLVTPGTPLTLTCTVSGIDLSSYAMGWVRQAPGKGLEWIGYIWSGGSADYASWAKGRFTISKTSTTVDLKIPSPTMEDTATYFCARGDAEYGDANGFAIWGPGTLVTVSL

SEQ ID NO: 20 - protein, 111 aa, synthetic construct
  REGION 1..111: note = clone 0279 VL
AYDMTQTPASVEVAVGGTVTIKCQASQSISNYLAWYQQKPGQPPKLLIYRASTLASGVSSRFKGSGSGTDFTLTISGVGCADAATYYCQQTLSTINIDNTFGGGTEVVVKR

SEQ ID NO: 21 - protein, 122 aa, synthetic construct
  REGION 1..122: note = clone 0291 VH
QSVEESGGRLVTPGTPLTLTCTVSGFSLSSGAMTWVRQAPGKGLEWIGYIWSGGSTDYASWAKGRFTISKTSTTVDLKITSPTTEDTATYFCARGYGADYPDFGDANGFDPWGPGTLVTVSS

SEQ ID NO: 22 - protein, 111 aa, synthetic construct
  REGION 1..111: note = clone 0291 VL
AYDMTQTPASVEVAVGGTVTIKCQASQSISSYLAWYQQKPGQPPKLLIYRASTLASGVSSRFKGSGSGTQFTLTISDLECADAATYYCQQGLSTINVDNTFGGGTEVVVKR

SEQ ID NO: 23 - protein, 122 aa, synthetic construct
  REGION 1..122: note = clone 0299 VH
QSMEESGGRLVTPGTPLTLTCTVSGFSLSSYAMSWVRQAPGKGLEWIGYIWSGGSTDYASWAKGRFTISKTSTTVDLKITSPTTEDTATYFCARRYGTSYPDYGDANGFDPWGPGTLVTVSS

SEQ ID NO: 24 - protein, 111 aa, synthetic construct
  REGION 1..111: note = clone 0299 VL
AYDMTQTPASVEVAVGGTVTIKCQASQSISSYLSWYQQKPGQRPKLLIYRASTLASGVSSRFKGSGSGTQFTLTISGVECADAATYYCQQCYSSSNVDNTFGGGTEVVVKR

SEQ ID NO: 25 - protein, 117 aa, synthetic construct
  REGION 1..117: note = clone 0300 VH
QSVEESGGRLVTPGTPLTLTCTASEFSLSNYYMSWVRQAPGKGLEYIGFIHTDGSTNYASWVNGRFTISRTSTTVDLKTTSLTTEDTATYFCCRYLVSGGTIAFDLWGPGTLVTVSS

SEQ ID NO: 26 - protein, 112 aa, synthetic construct
  REGION 1..112: note = clone 0300 VL
ADVVMTQTPASVEAAVGGTVTIKCQASQSISSYFAWYQQKPGQPPKLLIYRASKLATGVPSRFKGSGSGTEFTLTISDLECADAATYYCQTCGSISTYGGAFGGGTEVVVKR

SEQ ID NO: 27 - protein, 121 aa, synthetic construct
  REGION 1..121: note = clone 302 VH
QSLEESGGRLVTPGGSLTLTCTVSGFSLSTYAMTWVRQAPGKGLEWIGIIYAGTDITWYASWAKGRFTISKTSTTVDLSITSPTTEDTATYFCAKVPYTGVSDYLYYFDLWGPGTLVTVSS

SEQ ID NO: 28 - protein, 111 aa, synthetic construct
  REGION 1..111: note = clone 0302 VL
AFEMTQTPSSVSEPVGGTVTIKCQASENIYSFLAWYQQKPGQPPKLLIYDASDLASGVPSRFKGSGSGTQFTLTISDLECADAATYYCQQGYSGNVILNAFGGGTEVVVKR

SEQ ID NO: 29 - protein, 122 aa, synthetic construct; identical to SEQ ID NO: 23
  REGION 1..122: note = clone 0304 VH

SEQ ID NO: 30 - protein, 111 aa, synthetic construct; identical to SEQ ID NO: 24
  REGION 1..111: note = clone 0304 VL

SEQ ID NO: 31 - protein, 118 aa, synthetic construct
  REGION 1..118: note = clone 0323 VH
QSVEESGGRLVTPGTPLPLTCTVSGFSLSNYAMGWFRQAPGKGLEWIGYIWSGGSTDYASWAKGRFTISKTSTTVDLKMTSLTTEDTATYFCARADTVYRDDYGWSLWGPGTLVTVSS

SEQ ID NO: 32 - protein, 111 aa, synthetic construct
  REGION 1..111: note = clone 0323 VL
AYDMTQTPASVEVAVGGTVTIKCQASQSISIYLAWYQQKPGQPPKLLIYKASTLASGVPSRFKGSGSGTEFTLTISDLECADAATYYCQQAYSYSNVDNVFGGGTEVVVKR

SEQ ID NO: 33 - protein, 120 aa, synthetic construct
  REGION 1..120: note = clone 0325 VH
QSVEESGGRLVTPGTPLTLTCTVSGFSLSNYNMGWVRQAPGKGLEWIGIISDAGSAWYASWVKGRFTISKTSTTVDLKITSPTTEDAATYFCARGDAAAYTTGTEFFSLWGPGTLVTVSS

SEQ ID NO: 34 - protein, 111 aa, synthetic construct
  REGION 1..111: note = clone 0325 VL
AYDMTQTPASVEVAVGGTVTIKCQASEDIESYLAWYQQKPGQRPKLLIYAVSTLASGVSSRFKGSGSGTEYTLTISGVQCADAATYYCQQGYSSSNLDNAFGGGTEVVVRR

SEQ ID NO: 35 - protein, 120 aa, synthetic construct
  REGION 1..120: note = clone 0329 VH
GVQCQSLEESGGGLVKPGGTLTLTCTVSGFSLNSYSMTWVRQAPGKGLEWIGYIWSGGSADYANWAKGRFTISKTSTTVDLKMTSLTAEDTATYFCGRGYDTTDNGFNLWGPGTLVTVSS

SEQ ID NO: 36 - protein, 111 aa, synthetic construct
  REGION 1..111: note = clone 0329 VL
AYDMTQTPASVEAAVGGTVTIKCQASQSITSYLAWYQQKPGQPPKLLIYRASTLASGVSSRFKGSGSGTQFTLTISGVECADAATYYCQQTYRYMNVDNVFGGGTEVVVKR

SEQ ID NO: 37 - protein, 123 aa, synthetic construct
  REGION 1..123: note = clone 0339 VH
GVQCQSVEESGGRLVTPGTPLTLTCTVSGIDLSSHAMTWVRQAPGKGLEWIGYIWSGGSADYASWAKGRFTISKTSSTTVDLKIPSPTTEDTATYFCARGADEYGDANGFNIWGPGTLVTVSL

SEQ ID NO: 38 - protein, 111 aa, synthetic construct
  REGION 1..111: note = clone 0339 VL
AYDMTQTPSSVEAAVGGTVTIKCQASQSISNYLAWYQQKPGQPPELLIYRASTLASGVSSRFRGSGSGTDFTLTISGVGCADAATYYCQQGLSSSYVDNTFGGGTEVVVKR

SEQ ID NO: 39 - protein, 123 aa, synthetic construct
  REGION 1..123: note = clone 0343 VH
GVQCQEQLKESGGGLVTPGTPLTLTCTASGFSLSIYYMSWVRQAPGKGLDWIGFIYVDSSSAHYASWAKGRFTISKTSTTVDLKITSPTTEDTATYFCARDVDSSYFWGFNLWGPGTLVTVSS

SEQ ID NO: 40 - protein, 112 aa, synthetic construct
  REGION 1..112: note = clone 0343 VL
ADVVMTQTPSPVSGAVGGTVTIKCQASQSIDSYLSWYQQKPGQPPKLLIYSASTLASGVSSRFKGSGSGTQFTLTISDLECADAATYYCQCTYYDSSSIGGFGGGTEVVVKR

SEQ ID NO: 41 - protein, 116 aa, synthetic construct
  REGION 1..116: note = clone 0346 VH
GVQCQSVEESGGRLVTPGTPLTLTCTVSGFSLSSNAINWVRQAPGKGLEWIGYIYTDGNTYYASWAKGRFTISKTSTTVDLKITSPTTEDTATYFCARGVGYSDLWGPGTLVTVSS

SEQ ID NO: 42 - protein, 110 aa, synthetic construct
  REGION 1..110: note = clone 0346 VL
DVVMTQTPSSVSEPVGGTVTINCQASENIYSSLDWYQQKPGQPPKLLIYSASNLASGVSSRFKGSRSGTEYTLTISDLECADAATYYCQCIDGGNRGKPFGGGTEVVVKR

SEQ ID NO: 43 - protein, 122 aa, synthetic construct
  REGION 1..122: note = clone 0348 VH
GVQCQSVEESGGRLVTPGTPLTLTCTASGFSLNSVYMSWVRQAPGKGLEWIGIIYASGSIYYASWAKGRFTISRTSSTTVDLKMTSLTTEDTATYFCVRSADYDSGMPFDLWGPGTLVTVSS

SEQ ID NO: 44 - protein, 111 aa, synthetic construct
  REGION 1..111: note = clone 0348 VL
AQVLTQTASSVSAAVGGTVTISCQSSQSVWNNNFLSWYQQKPGQPPKLLIYLASTLASGVPSRFKGSGSGTQFTLTISDLECDDAATYYCAGVYSDNIFAFGGGTEVVVKR

SEQ ID NO: 45 - protein, 121 aa, synthetic construct
  REGION 1..121: note = clone 0349 VH
GVQCQSVEESGGRLVTPGTPLTLTCTVSGFSLSSSYMAWVRQAPGKGLEYIGFIHTDGSTYYATWVNGRFTISRTSTTVTLKMTSLTTEDTATYFCARYLVGSGAVAFDLWGPGTLVTVSS

SEQ ID NO: 46 - protein, 112 aa, synthetic construct
  REGION 1..112: note = clone 0349 VL
ADVVMTQTPASVEAAVGGTVTIKCQASQSISSYFAWYQQKPGQPPKLLIYRASNLATGVPSRFKGSGSGTEFTLTISDLECADAATYYCQSCGSISTYGGAFGGGTEVVVKR

SEQ ID NO: 47 - protein, 121 aa, synthetic construct
  REGION 1..121: note = clone 0350 VH
GVQCQSVEESGGRLVTPGTPLTLTCTVSGFSLSSYAMGWVRQAPGKGLEWIGYIWSGGSTDYATWAKGRFTISKTSTTVDLSITSPTTEDTAAYFCVRSAGSGDAMGFNLWGPGTLVTVSS

SEQ ID NO: 48 - protein, 111 aa, synthetic construct
  REGION 1..111: note = clone 0350 VL
AYDMTQTPASVSEPVGGTVTIKCQASQSISSYLSWYQQKPGQPPKLLIYRASTLESGVPSRFKGSGSGTEFTLTISDLECADAATYYCQQGLSVIHVDNTFGGGTEVVVKR

SEQ ID NO: 49 - protein, 122 aa, synthetic construct
  REGION 1..122: note = clone 0411 VH
QSVEESGGRLVTPGTPLTLTCTVSGFSLSNYAMTWVRQAPGKGLEWIGYIWSGGSTDYATWAKGRFTISKTSTTVDLKITSPTTEDTATYFCARGYGAGYPDYGDANGFDPWGPGTLVTVSS

SEQ ID NO: 50 - protein, 111 aa, synthetic construct
  REGION 1..111: note = clone 0411 VL
AYDMTQTPASVSAAVGGTVSINCQASQSISGYLSWYQQKPGQRPKLLIYRASTLASGVSSRFKGSGSGTQFTLTISGVECADAATYYCQQGYSIINVDNTFGGGTEVVVKR

SEQ ID NO: 51 - protein, 120 aa, synthetic construct
  REGION 1..120: note = clone 0431 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREESSYISGFDYWGQGTLVTVSS

SEQ ID NO: 52 - protein, 107 aa, synthetic construct
  REGION 1..107: note = clone 0431 VL
SSELTQDPAVSVALGQTVRITCQGDSLRSYYASWYQQKPGQAPVLVIYGKNNRPSGIPDRFSGSSSGNTASLTITGAQAEDEADYYCNSRDDYGDGVFGGGTKLTVL

SEQ ID NO: 53 - protein, 120 aa, synthetic construct
  REGION 1..120: note = clone 0432 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREESWWLSGLDYWGQGTLVTVSS

SEQ ID NO: 54 - protein, 107 aa, synthetic construct
  REGION 1..107: note = clone 0432 VL
SSELTQDPAVSVALGQTVRITCQGDSLRSYYASWYQQKPGQAPVLVIYGKNNRPSGIPDRFSGSSSGNTASLTITGAQAEDEADYYCNSMTESMNYVFGGGTKLTVL

SEQ ID NO: 55 - protein, 120 aa, synthetic construct
  REGION 1..120: note = clone 0433 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREESTWFSGFDYWGQGTLVTVSS

SEQ ID NO: 56 - protein, 108 aa, synthetic construct
  REGION 1..108: note = clone 0433 VL
SSELTQDPAVSVALGQTVRITCQGDSLRSYYASWYQQKPGQAPVLVIYGKNNRPSGIPDRFSGSSSGNTASLTITGAQAEDEADYYCNSIDSSGNHDVFGGGTKLTVL

SEQ ID NO: 57 - protein, 120 aa, synthetic construct
  REGION 1..120: note = clone 0434 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREYPWYIWSYDYWGQGTLVTVSS

SEQ ID NO: 58 - protein, 108 aa, synthetic construct
  REGION 1..108: note = clone 0434 VL
SSELTQDPAVSVALGQTVRITCQGDSLRSYYASWYQQKPGQAPVLVIYGKNNRPSGIPDRFSGSSSGNTASLTITGAQAEDEADYYCNSPDSIAYGVVFGGGTKLTVL

SEQ ID NO: 59 - protein, 119 aa, synthetic construct
  REGION 1..119: note = clone 0435 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARESYFLSGFDYWGQGTIVTVSS

SEQ ID NO: 60 - protein, 107 aa, synthetic construct
  REGION 1..107: note = clone 0435 VL
SSELTQDPAVSVALGQTVRITCQGDSLRSYYASWYQQKPGQAPVLVIYGKNNRPSGIPDRFSGSSSGNTASLTITGAQAEDEADYYCNSEDSESNLVFGGGTKLTVL

SEQ ID NO: 61 - protein, 118 aa, synthetic construct
  REGION 1..118: note = clone 0441 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSWFGEWMDYWGQGTLVTVSS

SEQ ID NO: 62 - protein, 107 aa, synthetic construct
  REGION 1..107: note = clone 0441 VL
SSELTQDPAVSVALGQTVRITCQGDSLRSYYASWYQQKPGQAPVLVIYGKNNRPSGIPDRFSGSSSGNTASLTITGAQAEDEADYYCNSIDSALDHVFGGGTKLTVL

SEQ ID NO: 63 - protein, 119 aa, synthetic construct
  REGION 1..119: note = clone 0445 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDSWYGWAFDYWGQGTLVTVSS

SEQ ID NO: 64 - protein, 107 aa, synthetic construct
  REGION 1..107: note = clone 0445 VL
SSELTQDPAVSVALGQTVRITCQGDSLRSYYASWYQQKPGQAPVLVIYGKNNRPSGIPDRFSGSSSGNTASLTITGAQAEDEADYYCNSRDMSLNVVFGGGTKLTVL

SEQ ID NO: 65 - protein, 119 aa, synthetic construct
  REGION 1..119: note = clone 0447 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARESFYGWAFDYWGQGTLVTVSS

SEQ ID NO: 66 - protein, 108 aa, synthetic construct
  REGION 1..108: note = clone 0447 VL
SSELTQDPAVSVALGQTVRITCQGDSLRSYYASWYQQKPGQAPVLVIYGKNNRPSGIPDRFSGSSSGNTASLTITGAQAEDEADYYCNSRDSTLIVVVFGGGTKLTVL

SEQ ID NO: 67 - protein, 119 aa, synthetic construct
  REGION 1..119: note = clone 0449 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARETWGWVAFDYWGQGTLVTVSS

SEQ ID NO: 68 - protein, 108 aa, synthetic construct
  REGION 1..108: note = clone 0449 VL
SSELTQDPAVSVALGQTVRITCQGDSLRSYYASWYQQKPGQAPVLVIYGKNNRPSGIPDRFSGSSSGNTASLTITGAQAEDEADYYCNSPDSSVNYMVFGGGTKLTVL

SEQ ID NO: 69 - protein, 120 aa, synthetic construct
  REGION 1..120: note = clone 0451 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREEYYYLSGFDYWGQGTLVTVSS

SEQ ID NO: 70 - protein, 107 aa, synthetic construct
  REGION 1..107: note = clone 0451 VL
SSELTQDPAVSVALGQTVRITCQGDSLRSYYASWYQQKPGQAPVLVIYGKNNRPSGIPDRFSGSSSGNTASITITGAQAEDEADYYCNSRDIIGDAVFGGGTKLTVL

SEQ ID NO: 71 - protein, 120 aa, synthetic construct
  REGION 1..120: note = clone 0452 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREAWDYLSSMDYWGQGTLVTVSS

SEQ ID NO: 72 - protein, 108 aa, synthetic construct
  REGION 1..108: note = clone 0452 VL
SSELTQDPAVSVALGQTVRITCQGDSLRSYYASWYQQKPGQAPVLVIYGKNNRPSGIPDRFSGSSSGNTASLTITGAQAEDEADYYCNSIDSEGNDIVFGGGTKLTVL

SEQ ID NO: 73 - protein, 117 aa, synthetic construct
  REGION 1..117: note = clone 0453 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDVYGHLDYWGQGTLVTVSS

SEQ ID NO: 74 - protein, 107 aa, synthetic construct
  REGION 1..107: note = clone 0453 VL
SSELTQDPAVSVALGQTVRITCQGDSLRSYYASWYQQKPGQAPVLVIYGKNNRPSGIPDRFSGSSSGNTASLTITGAQAEDEADYYCNSIDLSMDIVFGGGTKLTVL

SEQ ID NO: 75 - protein, 121 aa, synthetic construct
  REGION 1..121: note = clone 0486 VH
EVQLQQSGPELEKPGASVKISCKASGYSFTGYNMNWVKQSNGERLEWIGSIDPFYGGTTYNQKFKDKATLTVDKPSSTAYMQLTSLTAEDSAVYYCAREGGNYYGPWFAYWGQGTLVTVSA

SEQ ID NO: 76 - protein, 113 aa, synthetic construct
  REGION 1..113: note = clone 0486 VL
DVLMTQTPLSLPVSLGDQASISCRSTQSVVHSDGITHLEWYLQKPGQSPKLLIYKVFNRFYGVPDRFSGSGSGTDFTLKISRVEAEDLGVYYCFQGSHVPWTFGGGTKLEIKR

SEQ ID NO: 77 - protein, 121 aa, synthetic construct
  REGION 1..121: note = clone 0487 VH
DVQLVESGGGLVQPGGSRKLSCAASGFTFSSFGMHWVRQAPEKGLEWVAYISSGSGTIYYADTVKGRFTISRDNPKNTLFLQMTTLRSEDTAMYYCARSYYSDFGHAMDYWGQGTSVTVSS

SEQ ID NO: 78 - protein, 113 aa, synthetic construct
  REGION 1..113: note = clone 0487 VL
DVVMTQTPLTLSVTIGQPASISCKSSQSLLYTIGKTYLNWLLQRPGQSPKRLIYLVSKLDSGVPDRFSGSGSGTDFTLKISRVEAEDLGVYYCFQSTHFPLTFGAGTKLELRR

SEQ ID NO: 79 - protein, 119 aa, synthetic construct
  REGION 1..119: note = clone 0488 VH
DVQLVESGGGLVQPGGSRKLSCAASGFTFSTFGMHWVRQTPERGLEWVAYISSGGTNIYYADPVKGRFTISRDNPKKTLFLQMTGLRSEDTAIYYCARDGFGNYWFAYWGQGTLVTVSA

SEQ ID NO: 80 - protein, 108 aa, synthetic construct
  REGION 1..108: note = clone 0488 VL
DIQMTQSPASLSATVGETVTITCRTSGNIHNYLAWYQQKQGKSPQLLVYNGQTLAEGVPSKFSGSGSGTQYSLKINSLQPEDFGSYFCHQYFYFPLTFGTGTKLEIKR

SEQ ID NO: 81 - protein, 120 aa, synthetic construct
  REGION 1..120: note = clone 0489 VH
EVQLQQSGTELVRPGALVRLSCKASDFNIKDYYLHWVKQRPEQGLEWVGWIDPETLNTIYGPKFQGKASITADTSSNTAYLQLSGLTSEDIAVYYCTSSTVISYWHFDVWGAGTSVTVSS

SEQ ID NO: 82 - protein, 108 aa, synthetic construct
  REGION 1..108: note = clone 0489 VL
DIVMTQSHKFMSTSVGDRVTFTCKASQDVRSAVAWYQQKAGQSPKLLIYSASYRYTGVPDRFTGSGSGTDFTFTISSVQAEDLSVYYCQQHYTTPLTFGAGTKLELKR

SEQ ID NO: 83 - protein, 121 aa, synthetic construct
  REGION 1..121: note = clone 0490 VH
DVQLVESGGGLVQPGGSRKLSCAASGFTFSSFGMHWVRQAPEKGLEWVAYISSGSGTIYYADTMKGRFTISRDNPKNTLFLQMTTLRSEDTAMYYCARSYYSDFGHAMDYWGQGTSVTVSS

SEQ ID NO: 84 - protein, 113 aa, synthetic construct; identical to SEQ ID NO: 78
  REGION 1..113: note = clone 0490 VL

SEQ ID NO: 85 - protein, 119 aa, synthetic construct
  REGION 1..119: note = clone 0491 VH
DVQLVESGGGLVQPGGSRKLSCAASGFTFSTFGMHWVRQTPERGLEWVAYISSGSTNIYYADPVKGRFTISRDNPKKTLFLQMTGLRSEDTAMYYCARDGFGNYWFAYWGQGTLVTVSA

SEQ ID NO: 86 - protein, 108 aa, synthetic construct; identical to SEQ ID NO: 80
  REGION 1..108: note = clone 0491 VL

SEQ ID NO: 87 - protein, 121 aa, synthetic construct
  REGION 1..121: note = clone 0492 VH
DVQLVESGGGLVQPGGSRKLSCAASGFTFSSFGMHWVRQAPEKGLEWVAYISSGSSTIYYADTVKGRFTISRDNPKNTLFLQMASLRSEDTAMYYCARSYYGNFGFAMDYWGQGTSVTVSS

SEQ ID NO: 88 - protein, 113 aa, synthetic construct
  REGION 1..113: note = clone 0492 VL
DVVMTQTPLTLSVTIGQPASISCKSSQSLLYTQGKTYLNWLLQRPGQSPKRLIYLVSKLDSGVPDRFSGSGSGTDFTLKISRVEAEDLGVYYCLQSTHFPLTFGAGTKLELKR

SEQ ID NO: 89 - protein, 119 aa, synthetic construct
  REGION 1..119: note = clone 0493 VH
DVQLVESGGGLVQPGGSRKLSCAASGFTFSTFGMHWVRQTPERGLEWVAYISSDSSNIYYADPVKGRFTISRDNPKKTLFLQMTGLRSEDTAIYYCARDGFGNYWFAYWGQGTLVTVSA

SEQ ID NO: 90 - protein, 108 aa, synthetic construct; identical to SEQ ID NO: 80
  REGION 1..108: note = clone 0493 VL

SEQ ID NO: 91 - protein, 119 aa, synthetic construct
  REGION 1..119: note = clone 0494 VH
EVQLQQSGAVLVKPGASVKLSCPASGFNIKDTYIHWVIQRPEQGLEWIGRIDPANGDTKCDPKFQVKATITADTSSNTAYLQLSSLTSEDTAVYFCVRDYLYPYYFDFWGQGTTLTVSS

SEQ ID NO: 92 - protein, 108 aa, synthetic construct
  REGION 1..108: note = clone 0494 VL
KIVMTQSPKSMSMSVGERVTLNCRASESVDTYVSWYQQKPEQSPELLIYGASNRYTGVPDRFTGSGSATDFTLTISSVQAEDLADYYCGQTYNYPLTFGAGTKLELKR

SEQ ID NO: 93 - protein, 118 aa, synthetic construct
  REGION 1..118: note = clone 0495 VH
EVQLQQSGPELVKPGASVKMSCRASGYSFPSYVVHWVKQKPGQGLEWIGYINPYTDGTEYNEKFKGKATLTSDKSSSTAYMELSSLTSEDSAVYYCARGFYYYSMDYWGQGTSVTVSS

SEQ ID NO: 94 - protein, 107 aa, synthetic construct
  REGION 1..107: note = clone 0495 VL
QIVLTQSPAIMSASPGEKVTLTCSANSSIRNMHWYQQKTGTSPKRWIYDTSNLASGVPSRFSGSGSGTSYSLTISSMEAEDAATYYCHQRSSFPYTFGGGTKLEIKR

SEQ ID NO: 95 - protein, 124 aa, synthetic construct
  REGION 1..124: note = clone 0496 VH
QVQLIQSGAELVRPGASVNLSCKASGYTFTDYYINWVKQRPGQGLEWIARIYPGSDITYYNEKFKGKATLTAEKSSSTAYMQLSSLTSEDSAVYFCARSPPHYVGNRYYALDYWGQGTSVTVSS

SEQ ID NO: 96 - protein, 108 aa, synthetic construct
  REGION 1..108: note = clone 0496 VL
DIQMTQSSSSFSVSLGDRVTVTCKTTEDIYNRLAWYQHKPGNAPRLLISGATGLETGVPSRFSGSGSGKDYTLTITSLQTEDVATYYCLQYWSTPYTFGGGTKLEIKR

SEQ ID NO: 97 - protein, 123 aa, synthetic construct
  REGION 1..123: note = clone 0497 VH
QVQLQQPGAEFVKPGASVKMSCKASGYTFTSYWITWVKQRPGQGLEWIGDIYPGSGSNKYNEKFKSKATLTVDTSSSTAYMHLSSLTSEDSAVYYCARERPTYYGSSAWFDYWGQGTLVTVSA

SEQ ID NO: 98 - protein, 108 aa, synthetic construct
  REGION 1..108: note = clone 0497 VL
DIKMTQSPSSMYASLGERVTITCKASQDINSYLNWFQQKPGKSPKTLIYRANRLVDGVPSRFSGSGSGQDYSLTISSLDYEDMGIYYCLQYDEFPYTFGGGTKLEIKR

SEQ ID NO: 99 - protein, 916 aa, synthetic construct [sequence omitted]

SEQ ID NO: 100 - protein, 916 aa, synthetic construct
  REGION 1..916: note = cyTfR-Fc(kih)-Avi
MGWSCIILFLVATATGVHSGLNDIFEAQKIEWHEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGSGGGGSLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVTNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 101 - protein, 954 aa, synthetic construct [sequence omitted]

SEQ ID NO: 102 - protein, 954 aa, synthetic construct
  REGION 1..954: note = cyTfR-SNAP tag
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVTNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEFTRYPYDVPDYASGDKDCEMKRTTLDSPLGKLELSGCEQGLHEIKLLGSGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 103 - DNA, 37 bp, synthetic construct
  misc_feature 1..37: note = rbHC.up
  source 1..37: mol_type = other DNA
aagcttgccaccatggagactgggctgcgctggcttc

SEQ ID NO: 104 - DNA, 21 bp, synthetic construct
  misc_feature 1..21: note = rbHCf.do
  source 1..21: mol_type = other DNA
ccattggtgagggtgcccgag

SEQ ID NO: 105 - DNA, 34 bp, synthetic construct
  misc_feature 1..34: note = rbLC.up
  source 1..34: mol_type = other DNA
aagcttgccaccatggacaygagggcccccactc

SEQ ID NO: 106 - DNA, 26 bp, synthetic construct
  misc_feature 1..26: note = rbLC.do
  source 1..26: mol_type = other DNA
cagagtrctgctgaggttgtaggtac

SEQ ID NO: 107 - DNA, 20 bp, synthetic construct
  misc_feature 1..20: note = BcPCR_FHLC_leader.fw
  source 1..20: mol_type = other DNA
atggacatgagggtccccgc

SEQ ID NO: 108 - DNA, 24 bp, synthetic construct
  misc_feature 1..24: note = BcPCR_huCkappa.rev
  source 1..24: mol_type = other DNA
gatttcaactgctcatcagatggc

SEQ ID NO: 109 - protein, 6 aa, synthetic construct
  REGION 1..6: note = clone 299 HVR-H1
FSLSSY

SEQ ID NO: 110 - skipped

SEQ ID NO: 111 - protein, 15 aa, synthetic construct
  REGION 1..15: note = clone 299 HVR-H2
YIWWSGGSTDYASWA

SEQ ID NO: 112 - protein, 14 aa, synthetic construct
  REGION 1..14: note = clone 299 HVR-H3
GTSYPDYGDANGFD

SEQ ID NO: 113 - protein, 7 aa, synthetic construct
  REGION 1..7: note = clone 299 HVR-L1
SQSISSY

SEQ ID NO: 114 - skipped

SEQ ID NO: 115 - protein, 9 aa, synthetic construct
  REGION 1..9: note = clone 299 HVR-L3
CYSSSNVDN

SEQ ID NO: 116 - protein, 7 aa, synthetic construct
  REGION 1..7: note = clone 494 HVR-H1
GFNIKDT

SEQ ID NO: 117 - skipped

SEQ ID NO: 118 - protein, 17 aa, synthetic construct
  REGION 1..17: note = clone 494 HVR-H2
RIDPANGDTKCDPKFQV

SEQ ID NO: 119 - protein, 8 aa, synthetic construct
  REGION 1..8: note = clone 494 HVR-H3
YLYPYYFD

SEQ ID NO: 120 - protein, 7 aa, synthetic construct
  REGION 1..7: note = clone 494 HVR-L1
SESVDTY

SEQ ID NO: 121 - skipped

SEQ ID NO: 122 - protein, 6 aa, synthetic construct
  REGION 1..6: note = clone 494 HVR-L3
TYNYPL